(19) United States
(12) Patent Application Publication
(10) Pub. No.: US 2025/0197391 A1
(43) Pub. Date: Jun. 19, 2025

(54) METHODS OF SYNTHESIZING EGFR INHIBITORS

(71) Applicant: Scorpion Therapeutics, Inc., Boston, MA (US)

(72) Inventors: Benjamin C. Milgram, Weston, MA (US); Benjamin Faraz Rahemtulla, Hoddesdon (GB); Joseph Marshall Bateman, Bedfordshire (GB); Eric P. A Talbot, Hitchin (GB); Thomas A. Mulhern, Portage, MI (US)

(21) Appl. No.: 18/849,887

(22) PCT Filed: Mar. 24, 2023
(86) PCT No.: PCT/US2023/016280

§ 371 Date: Sep. 23, 2024

Related U.S. Application Data
(60) Provisional application No. 63/435,108, filed on Dec. 23, 2022.
(60) Provisional application No. 63/422,645, filed on Nov. 4, 2022.
(60) Provisional application No. 63/323,249, filed on Mar. 24, 2022.

(51) Int. Cl. C07D 471/04

(52) U.S. Cl. CPC C07D 471/04

(57) ABSTRACT

This disclosure features methods for preparing tetrahydro-4H-pyrrolo[3,2-c]pyridin-4-one-containing chemical entities (e.g., a compound or a pharmaceutically acceptable salt thereof) that inhibit epidermal growth factor receptor (EGFR, ERBB 1) and/or Human epidermal growth factor receptor 2 (HER2, ERBB2) as well as synthetic intermediates useful for the preparation of said chemical entities. The methods include, for example, improved methods for synthesizing compounds of formula (I) as described herein.

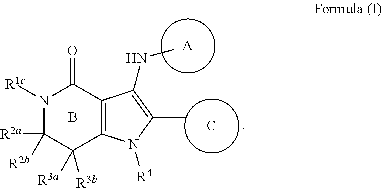

### CROSS REFERENCE TO RELATED APPLICATIONS

This application claims the benefit of U.S. Provisional Application No. 63/323,249, filed on Mar. 24, 2022, 63/422,645, filed on Nov. 4, 2022 and 63/435,108, filed on Dec. 23, 2022, which are incorporated herein by reference in their entirety.

### TECHNICAL FIELD

This disclosure features methods for preparing tetrahydro-4H-pyrrolo[3,2-c]pyridin-4-one-containing chemical entities (e.g., a compound or a pharmaceutically acceptable salt thereof) that inhibit epidermal growth factor receptor (EGFR, ERBB1) and/or Human epidermal growth factor receptor 2 (HER2, ERBB2) as well as synthetic intermediates useful for the preparation of said chemical entities. The methods include, for example, improved methods for synthesizing compounds of formula (I) as described herein.

### BACKGROUND

Epidermal growth factor receptor (EGFR, ERBB1) and Human epidermal growth factor receptor 2 (HER2, ERBB2) are members of a family of proteins which regulate cellular processes implicated in tumor growth, including proliferation and differentiation. Several investigators have demonstrated the role of EGFR and HER2 in development and cancer (Reviewed in Salomon, et al., Crit. Rev. Oncol. Hematol. (1995) 19:183-232, Klapper, et al., Adv. Cancer Res. (2000) 77, 25-79 and Hynes and Stern, Biochim. Biophys. Acta (1994) 1198:165-184). EGFR overexpression is present in at least 70% of human cancers, such as non-small cell lung carcinoma (NSCLC), breast cancer, glioma, and prostate cancer. HER2 overexpression occurs in approximately 30% of all breast cancer. It has also been implicated in other human cancers including colon, ovary, bladder, stomach, esophagus, lung, uterus and prostate. HER2 overexpression has also been correlated with poor prognosis in human cancer, including metastasis, and early relapse.

Tetrahydro-4H-pyrrolo[3,2-c]pyridin-4-one-containing chemical entities that inhibit epidermal growth factor receptor and/or Human epidermal growth factor receptor 2 are described in, e.g., PCT/US2021/051504, filed on Sep. 22, 2021; PCT/US2021/054191, filed on Oct. 8, 2021; and PCT/US2021/057348, filed on Oct. 8, 2021, each of which is incorporated by reference in its entirety.

### SUMMARY

This disclosure features methods for preparing tetrahydro-4H-pyrrolo[3,2-c]pyridin-4-one-containing chemical entities (e.g., a compound or a pharmaceutically acceptable salt thereof) that inhibit epidermal growth factor receptor (EGFR, ERBB1) and/or Human epidermal growth factor receptor 2 (HER2, ERBB2) as well as synthetic intermediates useful for the preparation of said chemical entities. The methods include, for example, improved methods for synthesizing compounds of formula (I) as described herein.

In one aspect, this disclosure features methods of preparing a compound of Formula (I), or a pharmaceutically acceptable salt thereof, which include contacting a compound of formula (II) with a compound of formula (III),

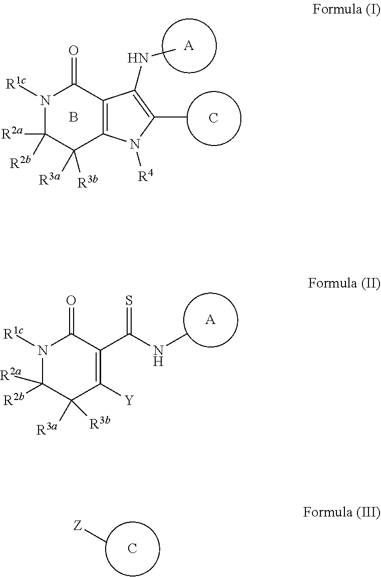

in which Y, Z, R, R^{2a}, R^{2b}, R^{3a}, R^{3b}, R⁴, ring A and ring C can be as defined anywhere herein.

In one aspect, this disclosure features compound of Formula (I), or a pharmaceutically acceptable salt thereof,

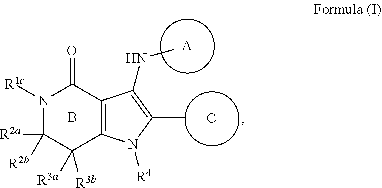

in which R^{1c}, R^{2a}, R^{2b}, R^{3a}, R^{3b}, R⁴, ring A and ring C can be as defined anywhere herein.

In another aspect, this disclosure features compound of Formula (I), or a pharmaceutically acceptable salt thereof,

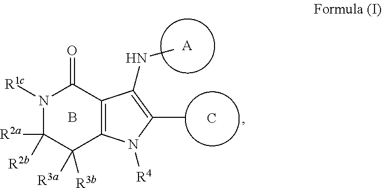

which is prepared by a process as described anywhere herein, and in which R^{1c}, R^{2a}, R^{2b}, R^{3a}, R^{3b}, R⁴, ring A and ring C can be as defined anywhere herein.

Procedures used heretofore to prepare the compounds described herein utilized oxidizing agents (e.g., peroxy acids, e.g., m-CPBA) in certain bond forming (e.g., cyclization) steps. Additionally, the oxidizing agents (e.g., peroxy acids, e.g., m-CPBA) were employed in at least stoichiometric amounts, and typically in excess. Advantageously, the inventors have surprisingly found that these bond forming (e.g., cyclization) steps can be carried out in the absence of oxidizing agents (e.g., peroxy acids, e.g., m-CPBA), thereby rendering the desired transformation more amenable to safer and more cost effective scale-up.

The disclosure may be more fully appreciated by reference to the following description, including the following glossary of terms and the concluding examples. It is to be appreciated that certain features of the disclosed compositions and methods which are, for clarity, described herein in the context of separate aspects, may also be provided in combination in a single aspect. Conversely, various features of the disclosed compositions and methods that are, for brevity, described in the context of a single aspect, may also be provided separately or in any sub-combination.

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “alkoxy” refers to an —O-alkyl radical (e.g., —OCH₃).

The term “alkylene” refers to a divalent alkyl (e.g., —CH₂—).

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

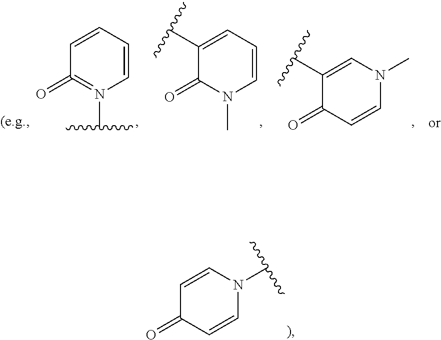

pyrimidone

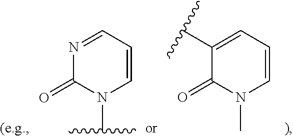

pyridazinone

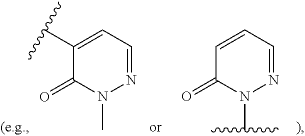

pyrazinone

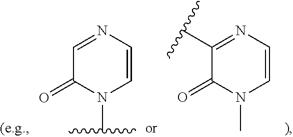

and imidazolone

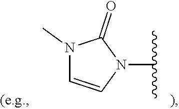 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl. As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

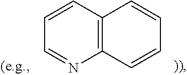

(ii) a single ring atom (spiro-fused ring systems)

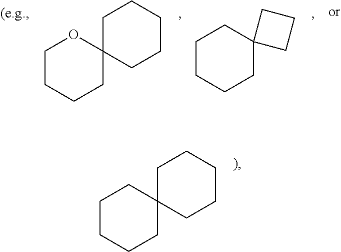

or (iii) a contiguous array of ring atoms (bridged ring systems having all bridge lengths>0)

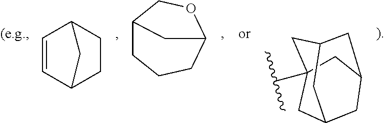

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

According to the disclosure, the compounds prepared by the methods described herein can be obtained as a single stereoisomer or a mixture of stereoisomers.

Compounds prepared by the methods described herein may also contain unnatural proportions of one, two, three, or more atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as, for example, tritium (³H), iodine-125 (¹²⁵I), fluorine-18 (¹⁸F), and/or carbon-14 (14C), or non-radioactive isotopes, such as deuterium (²H), carbon-13 (¹³C), and/or nitrogen-15 (¹⁵N). Such isotopic variations can provide additional utilities to those described elsewhere within this application. For instance, isotopic variants of the compounds of the invention may find additional utility, including but not limited to, as diagnostic and/or imaging reagents, or as cytotoxic/radiotoxic therapeutic agents. Additionally, isotopic variants of the compounds of the invention can have altered pharmacokinetic and pharmacodynamic characteristics which can contribute to enhanced safety, tolerability or efficacy during treatment. All isotopic variations of the compounds of the present invention, whether radioactive or not, are intended to be encompassed within the scope of the present invention.

The details of one or more embodiments of the subject matter claimed are set forth in the accompanying drawings and the description below. Other features, and advantages of the invention will be apparent from the description and drawings, and from the claims.

### DESCRIPTION OF DRAWINGS

FIGS. 1A-1C show the ¹HNMR and LCMS spectra of control experiments investigating compatibility of 5a and 6a with NH₄OAc.

FIG. 2 shows the ¹H NMR spectrum of compound 101.

FIG. 3 shows the ¹H NMR spectrum of compound 102.

FIG. 4A. shows the LC-MS spectrum of compound 102. FIG. 4B shows the powder of compound 102

FIGS. 5A-5CC show the ¹H NMR, ¹³C NMR, 19F NMR, and NOESY NMR of the compounds synthesized in examples 7-45.

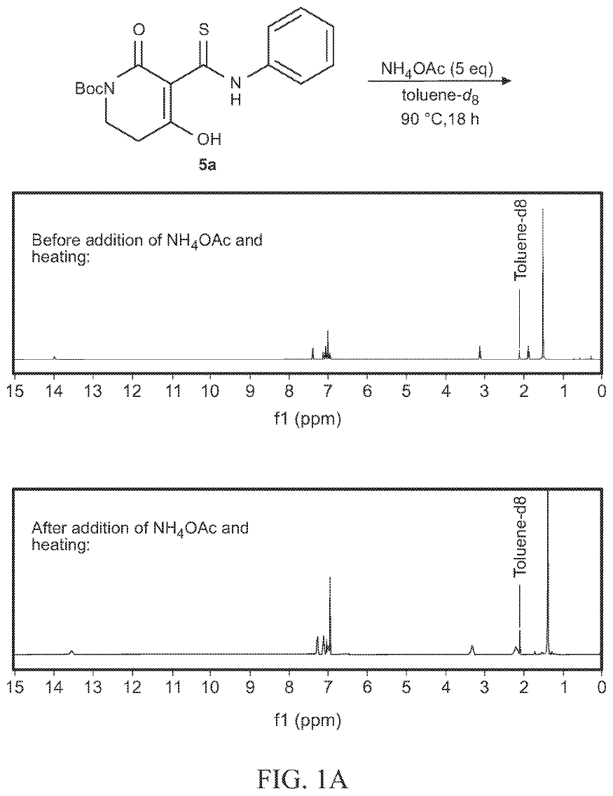

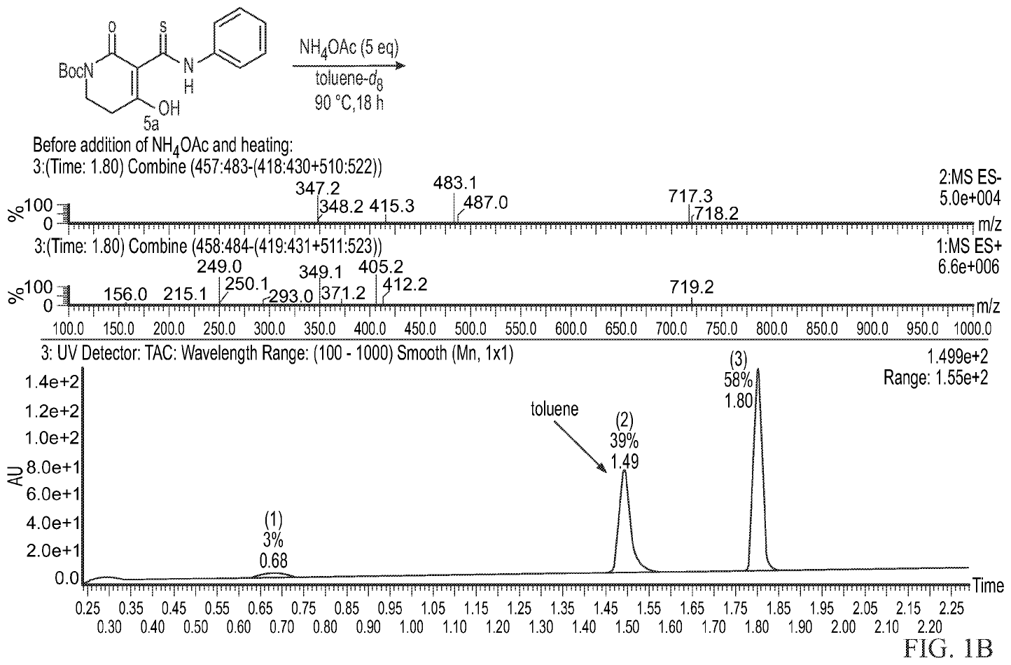

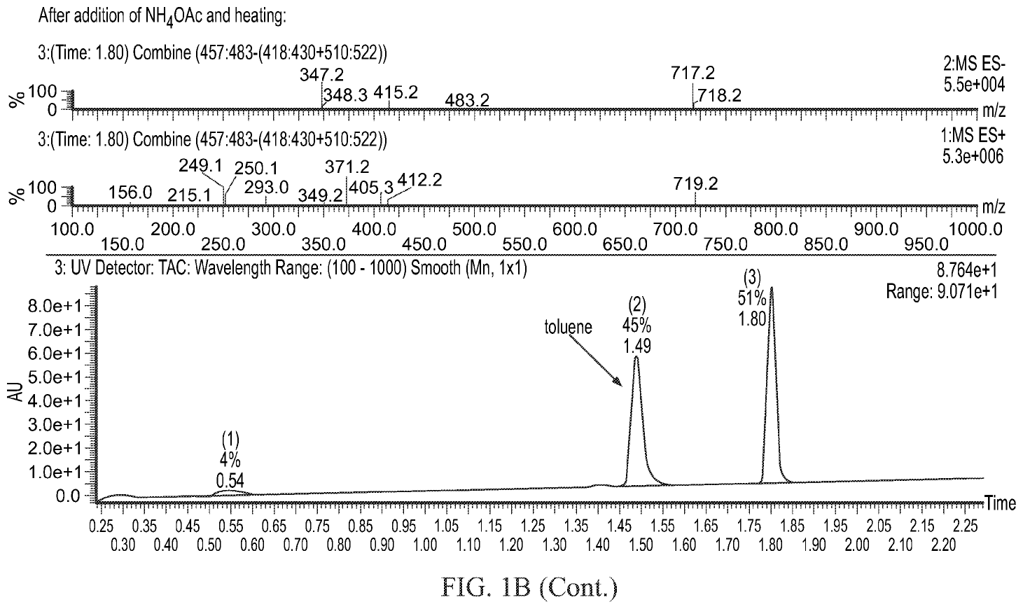

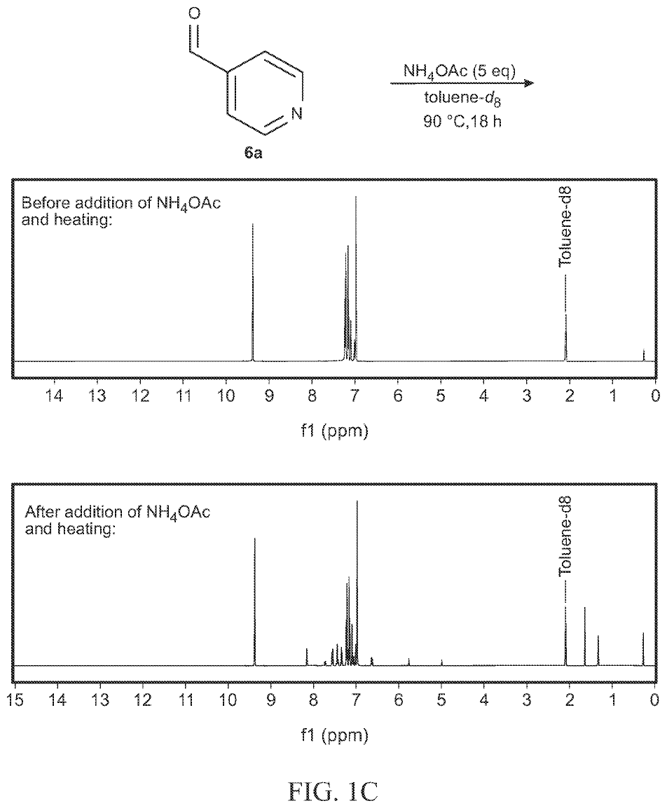

### DETAILED DESCRIPTION

This disclosure features methods for preparing tetrahydro-4H-pyrrolo[3,2-c]pyridin-4-one-containing chemical entities (e.g., a compound or a pharmaceutically acceptable salt thereof) that inhibit epidermal growth factor receptor (EGFR, ERBB1) and/or Human epidermal growth factor receptor 2 (HER2, ERBB2) as well as synthetic intermediates useful for the preparation of said chemical entities. The methods include, for example, improved methods for synthesizing compounds of formula (I) as described herein.

### Preparation of Compounds of Formula (I)

In one aspect, this disclosure features methods of preparing a compound of Formula (I), or a pharmaceutically acceptable salt thereof, which include contacting a compound of formula (II) with a compound of formula (III),

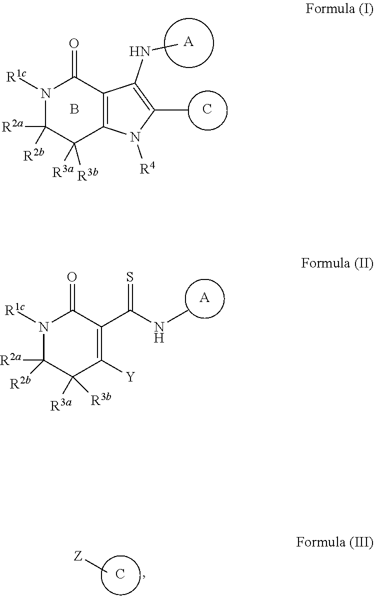

in which Y, Z, R^{1c}, R^{2a}, R^{2b}, R^{3a}, R^{3b}, R⁴, ring A and ring C can be as defined anywhere herein. By way of example:
Y is selected from —OH or —NH₂;
Z is selected from
   —C(═O)H; or
   —CH(R)₂ wherein each R independently selected from halo, alkoxy, OH or SO₃M (M=Li, Na, K or NH₄⁺), provided that when one R is OH, the other R cannot be halo, alkoxy or OH;
R^{1c} is selected from H, and R^{d};
each of R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g};
two of variables R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d};
Ring C is selected from the group consisting of:

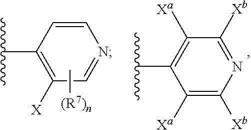

wherein:
   each X^{b} is independently X, R^{c}, or H; and
   each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X and further optionally substituted with from 1-4 R^{c};
2-pyridonyl or 4-pyridonyl, each optionally substituted with X and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{e};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is substituted with X and optionally substituted with from 1-4 substituents independently selected from the group consisting of oxo and R⁷;
bicyclic C₅₋₁₀ cycloalkyl or C₅₋₁₀ cycloalkenyl, each of which is substituted with X and is optionally substituted with from 1-4 substituents independently selected from the group consisting of oxo and R⁷;
heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is substituted with X and optionally substituted with from 1-4 substituents independently selected from the group consisting of oxo and R⁷; and
C₁₀ or C₁₄ aryl substituted with X and optionally substituted with from 1-4 R⁷;
X is X*, wherein X* is selected from halo, triflate, tosylate, or mesylate; or
X is X¹;
each R⁷ is an independently selected R^{c}; n is 0, 1, 2, or 3;
X¹ is selected from the group consisting of: (a) —O-L¹-R⁵; and (b)

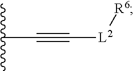

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

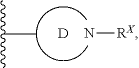

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a},
R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
   H;
   halo;
   —OH;
   —NR^{e}R^{f};
   —R^{g};
   R^{w}
   -L⁶-R^{g};
   —R^{g2}—R^{W} or —R^{g2}—R^{Y};
   -L⁶-R^{g2}—R^{W} or -L⁶-R^{g2}—R^{Y}; and
   —C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—; and
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system;
and
R^{X} is C(═O)(C₁₋₆ alkyl) or S(O)₂(C₁₋₆ alkyl), each of which is optionally substituted with from 1-6 R^{a}; and
R^{Y} is selected from the group consisting of: —R^{g} and -(L^{g})_{g}-R^{g}.
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*;
C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl);
—NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c}.
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

In another aspect, this disclosure features methods of preparing a compound of Formula (I), or a pharmaceutically acceptable salt thereof, the method comprising contacting a compound of formula (II) with a compound of formula (III),

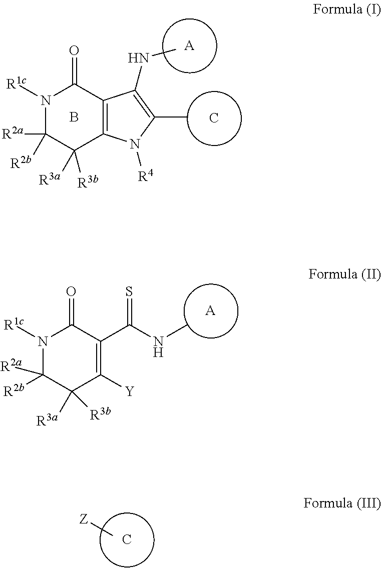

wherein
Y is selected from —OH or —NH₂;
Z is selected from
   —C(═O)H; or
   —CH(R)₂ wherein each R independently selected from halo, alkoxy, OH or SO₃M (M=Li, Na, K or NH₄⁺), provided that when one R is OH, the other R cannot be halo, alkoxy or OH;
R^{1c} is selected from H, and R^{d};
each of R^{2a}, R^{2b}, R^{3a}, and R^{3A} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g};
two of variables R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W}.
Ring A is R^{g};
R⁴ is selected from the group consisting of: H and R^{d};
Ring C is selected from the group consisting of:

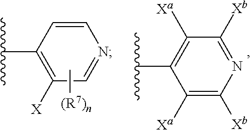

wherein:
   each X^{b} is independently X, R^{c}, or H; and
   each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X and further optionally substituted with from 1-4 R^{c};
2-pyridonyl or 4-pyridonyl, each optionally substituted with X and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c};
   bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷;
   bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷;
   heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷; and
   C₁₀ or C₁₄ aryl optionally substituted with X and optionally substituted with from 1-4 R⁷;

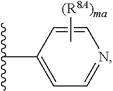

wherein
   ma is 0, 1, 2, or 3;
   R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A};
   R^{5A} is selected from hydroxy, halogen, cyano, C₁₋₄-alkyl, C₁₋₄-alkoxy, C₁₋₄ haloalkyl or C₁₋₄ haloalkoxy;
   R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{SA};
   R^{11A} is independently selected from C₁₋₄ alkyl, C₃₋₆ cycloalkyl, C₁₋₄ haloalkyl or C₃₋₆ halocycloalkyl;

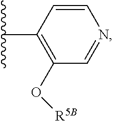

wherein
   R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{5A}; or
   R^{5B} is R^{6B}—CH₂—;
   R^{6B} is selected from

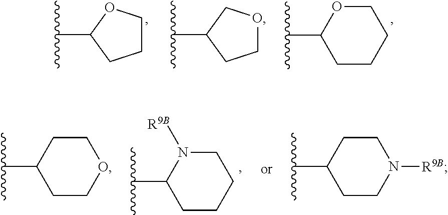

R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy;

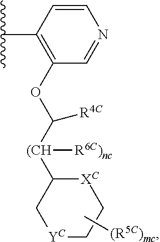

wherein
   R^{4C} is selected from hydrogen or methyl;
   R^{6C} is selected from hydrogen, C₁₋₃ alkyl, C₁₋₃ haloalkyl;
   nc is 0 or 1;
   X^{C} is NR^{7C} or O;
   Y^{C} is NR^{8C} or O;
   R^{7C} is methyl;
   R^{8C} is selected from methyl, 2,2,2-trifloethyl, or 2,2-difluoroethyl;
   R^{5C} is selected from hydrogen or methyl, wherein R^{5C} being attached to any carbon atom of the ring comprising X^{C} and Y^{C};
   mc is 0, 1, 2, or 3;

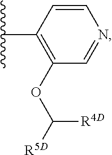

wherein
R^{4D} is selected from hydrogen or methyl;
R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R^{6D} and wherein each azetidinyl is substituted at the nitrogen with R^{8D}; OR
R^{5D} is selected from the group consisting of

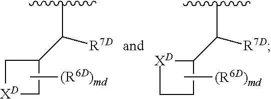

R^{6D} is selected from fluoro or C₁₋₃ alkyl;
md is selected from 0, 1, 2, or 3;
R^{7D} is selected from hydrogen, C₁₋₃ alkyl, or C₁₋₃ haloalkyl;
X^{D} is NR^{8D} of O;
R^{8D} is selected from C₁₋₃ alkyl or C₂₋₃ haloalkyl;
X is X*, wherein X* is selected from halo, triflate, tosylate, or mesylate; or
X is X¹;
each R⁷ is an independently selected R^{c}; n is 0, 1, 2, or 3;
X¹ is selected from the group consisting of: (a) —O-L¹-R⁵; and (b)

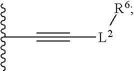

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

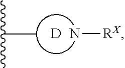

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
   H;
   halo;
   —OH;
   —NR^{e}R^{f};
   —R^{g};
   —R^{W}
   -L⁶-R^{g};
   —R^{g2}—R^{W} or —R^{g2-}R^{Y};
   -L⁶-R^{g2}—R^{W} or -L⁶-R^{g2}—R^{Y}; and
   —C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—; and
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a}; and
R^{Y} is selected from the group consisting of: —R^{g} and -(L^{g})_{g}-R^{g}.
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), 0, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

In another aspect, this disclosure features methods of preparing a compound of Formula (I), or a pharmaceutically acceptable salt thereof, the method comprising contacting a compound of formula (II) with a compound of formula (III),

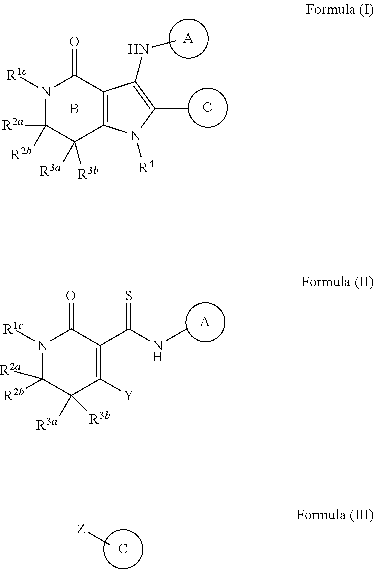

wherein
Y is selected from —OH or —NH₂;
Z is selected from
   —C(═O)H; or
   —CH(R)₂ wherein each R independently selected from halo, alkoxy, OH or SO₃M (M=Li, Na, K or NH₄⁺), provided that when one R is OH, the other R cannot be halo, alkoxy or OH;
R^{1c} is selected from H, and R^{d};
each of R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; two of variables R^{2a}, R^{2b}, R^{3a}, and R^{3A}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d}.
Ring C is selected from the group consisting of:

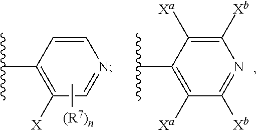

wherein:
   each X^{b} is independently X, R^{c}, or H; and
   each X^{a} is independently selected from the group consisting of H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X and further optionally substituted with from 1-4 R^{c};
2-pyridonyl or 4-pyridonyl, each optionally substituted with X and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c};
   bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷;
   bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷;
   heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷; and
   C₁₀ or C₁₄ aryl optionally substituted with X and optionally substituted with from 1-4 R⁷;
X is X*, wherein X* is selected from halo, triflate, tosylate, or mesylate; or
X is X¹;
each R⁷ is an independently selected R^{c}; n is 0, 1, 2, or 3;
X¹ is selected from the group consisting of: (a) —O-L¹-R⁵; and (b)

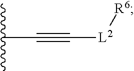

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

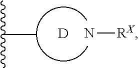

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
   H;
   halo;
   —OH;
   —NR^{e}R^{f};
   —R^{g};
   —R^{w}
   -L⁶-R^{g};
   —R^{g2}—R^{W} or —R^{g2}—R^{Y};
   -L⁶-R^{g2}—R^{W} or -L⁶-R^{g2}—R^{Y}; and
   —C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—; and
R^{W} is -L^{W}-W,
wherein L^{W} is C(═O), S(O)₁₋₂, OC(═O)*, NHC(═O)*, NR^{d}C(═O)*, NHS(O)ₜ₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a}; and
R^{Y} is selected from the group consisting of: —R and -(L^{g})_{g}-R^{g}.
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c};
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

In another aspect, this disclosure features methods of preparing a compound of Formula (I), or a pharmaceutically acceptable salt thereof, the method comprising contacting a compound of formula (II) with a compound of formula (III),

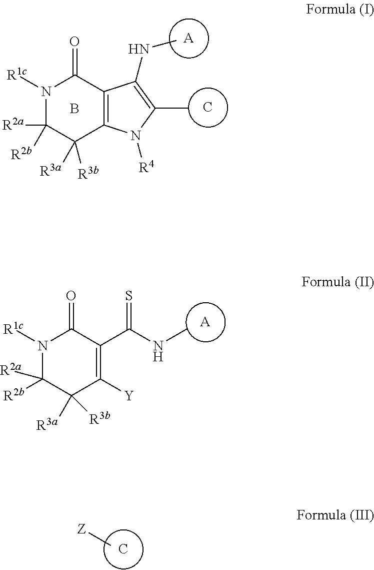

wherein
Y is selected from —OH or —NH₂;
Z is selected from
   —C(═O)H; or
   —CH(R)₂ wherein each R independently selected from halo, alkoxy, OH or SO₃M (M=Li, Na, K or NH₄⁺), provided that when one R is OH, the other R cannot be halo, alkoxy or OH;
R^{1c} is selected from H, and R^{d}.
each of R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g};
two of variables R^{2a}, R^{2b}, R^{3a}, and R^{3b}, together with the Ring B ring atoms to which each is attached, form a fused saturated or unsaturated ring of 3-12 ring atoms;
   wherein from 0-2 of the ring atoms are each an independently selected heteroatom (in addition to —N(R^{1c})— when —N(R^{1c})— forms part of the fused saturated or unsaturated ring), wherein each of the independently selected heteroatoms is selected from the group consisting of N, NH, N(R^{d}), O, and S(O)₀₋₂; and
   wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d};
Ring C is selected from the group consisting of:

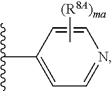

wherein
   ma is 0, 1, 2, or 3;
   R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A}.
   R^{IA} is selected from hydroxy, halogen, cyano, C₁₋₄-alkyl, C₁₋₄-alkoxy, C₁₋₄ haloalkyl or C₁₋₄ haloalkoxy;
   R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{5A};
   R^{11A} is independently selected from C₁₋₄ alkyl, C₃₋₆ cycloalkyl, C₁₋₄ haloalkyl or C₃₋₆ halocycloalkyl;

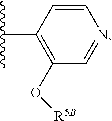

wherein
   R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{5A}; or
   R^{5B} is R^{6B}—CH₂—;
   R^{6B} is selected from

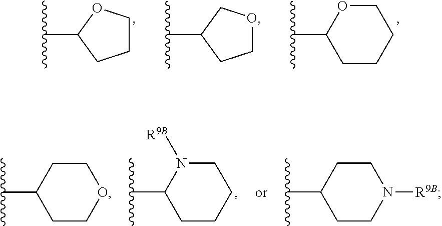

R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy;

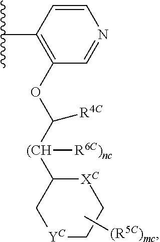

wherein
   R^{4C} is selected from hydrogen or methyl;
   R^{6C} is selected from hydrogen, C₁₋₃ alkyl, C₁₋₃ haloalkyl;
   nc is 0 or 1;
   X^{C} is NR^{7C} or O;
   Y^{C} is NR^{8C} or O;
   R^{7C} is methyl;
   R^{5C} is selected from methyl, 2,2,2-trifloethyl, or 2,2-difluoroethyl;
   R^{5C} is selected from hydrogen or methyl, wherein R^{5C} being attached to any carbon atom of the ring comprising X^{C} and Y^{C};
   me is 0, 1, 2, or 3;

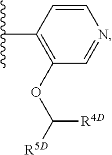

wherein
   R^{4D} is selected from hydrogen or methyl;
   R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R^{6D} and wherein each azetidinyl is substituted at the nitrogen with R^{8D}; OR
   R^{5D} is selected from the group consisting of

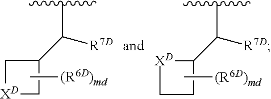

R^{6D} is selected from fluoro or C₁₋₃ alkyl;
md is selected from 0, 1, 2, or 3;
R⁷) is selected from hydrogen, C₁₋₃ alkyl, or C₁₋₃ haloalkyl;
X^{D} is NR^{8′} of O;
R^{8D} is selected from C₁₋₃ alkyl or C₂₋₃ haloalkyl;
X is X*, wherein X* is selected from halo, triflate, tosylate, or mesylate; or
X is X¹;
each R⁷ is an independently selected R^{c}; n is 0, 1, 2, or 3;
X¹ is selected from the group consisting of: (a) —O-L¹-R⁵; and (b)

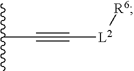

L¹ and L² are independently selected from the group consisting of: a bond and C₁₋₁₀ alkylene optionally substituted with from 1-6 R^{a};
R⁵ is selected from the group consisting of:
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
   C₆₋₁₀ aryl optionally substituted with from 1-4 R^{c};
   C₃₋₁₀ cycloalkyl or C₃₋₁₀ cycloalkenyl, each optionally substituted with from 1-4 substituents each independently selected from the group consisting of: oxo and R^{c};

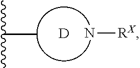

wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a};
—R^{W}
—R^{g2}—R^{W} or —R^{g2}—R^{Y};
-L⁵-R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
   H;
   halo;
   —OH,
   —NR^{e}R^{f};
   —R^{g};
   —R^{w}
   -L⁶-R^{g};
   —R^{g2}—R^{W} or —R^{g2}—R^{Y};
   -L⁶-R^{g2}—R^{W} or -L⁶-R^{g2}—R^{Y}; and
   C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—; and
R^{W} is L^{W}-W,
wherein L^{W} is C(═O), S(O)₁₋₂, OC(═O)*, NHC(═O)*, NR^{d}C(═O)*, NHS(O)ₜ₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a}; and
R^{Y} is selected from the group consisting of: —R^{g} and -(L^{g})_{g}-R^{g}.
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*; C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
   C₃₋₁₀ cycloalkyl or C₃₋₁₀ cycloalkenyl, each of which is optionally substituted with from 1-4 substituents independently selected from the group consisting of oxo and R^{c}.
   heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
   C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a}.
each g is independently 1, 2, or 3;
each R^{g2} is a divalent R^{g} group; and
each occurrence of R′ and R″ is independently selected from the group consisting of: H; —OH; and C₁₋₄ alkyl.

Ring A in formula (I) can be as defined anywhere herein.

Variables R^{1c}, R^{2a}, R^{2b}, R^{3a}, and R^{3b} in formula (I) can be as defined anywhere herein.

Ring C in formula (I) can be as defined anywhere herein.

In some embodiments, the contacting the compound of formula (II) with the compound of formula (III) is carried out in the presence of a nitrogen source.

In certain embodiments, the nitrogen source is ammonia or derivative thereof.

In certain embodiments, the nitrogen source is in the form of a salt.

In certain embodiments, the nitrogen source is an ammonium salt.

Non-limiting examples of the nitrogen sources include NH₄OAc, NH₃·H₂O, NH₄CO₂H, NH₄OBz, NH₄Cl, (NH₄)₂SO₄, (NH₄)₂HPO₄, NH₄H₂PO₄, NH₄OTf, NH₄HCO₃, (NH₄)₂CO₃, NH₄CO₂CF₃, NH₄BF₄, ammonium citrate dibasic, NH₄Br, ammonium carbamate, or any combination thereof. Other examples include primary alkyl and cycloalkyl amines, e.g., (C₁-C₆ alkyl)-NH₂ and (C₃-C₆ cycloalkyl)-NH₂.

For example, the nitrogen source can be NH₄OAc.

In some embodiments, the molar ratio of the nitrogen source to the compound of formula (III) is from about 2:1 to about 8:1.

In certain embodiments, the molar ratio of the nitrogen source to the compound of formula (III) is from about 4:1 to about 6:1.

In certain embodiments, the molar ratio of the nitrogen source to the compound of formula (III) is about 4.5:1; 4.6:1; 4.7:1; 4.8:1; 4.9:1; 5:1; 5.1:1; 5.2:1; 5.3:1; 5.4:1; or 5.5:1,

For example, the molar ratio of the nitrogen source to the compound of formula (III) can be about 5:1.

In some embodiments an equivalent amount or an excess amount of the compound of formula (III) relative to the compound of formula (II) is employed. In certain embodiments, the molar ratio of the compound of formula (III) relative to the compound of formula (II) is from about 1:1 to about 3:1, e.g., about 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, or about 3:1. In certain embodiments, the molar ratio of the compound of formula (III) relative to the compound of formula (II) is about 1.3:1 or about 1.5:1 or about 2:1. In certain embodiments, the compound of formula (III) is added portion-wise, e.g., over a period of from about 2 hours to about 4 hours.

In some embodiments, contacting the compound of formula (II) with the compound of formula (III) is carried out in the presence of a suitable solvent (e.g., a suitable organic solvent). Mixtures of solvents (e.g., organic solvents) can also be employed.

In some embodiments, the solvent is an aprotic solvent.

In some embodiments, the aprotic solvent is a non-polar aprotic solvent.

In certain embodiments, the non-polar aprotic solvent is an aromatic hydrocarbon solvent. Aromatic hydrocarbon solvents include, without limitation, toluene, anisole, xylenes (e.g., mixed xylenes (BTEX)), trifluorotoluene, benzene, chlorobenzene, 1, 2-dichlorobenzene, 1, 2-difluorobenzene, hexafluorobenzene, ethylbenzene, and high flash aromatic naphthas. For example, the aromatic hydrocarbon solvent can be toluene.

In certain embodiments, the non-polar aprotic solvent is a non-aromatic hydrocarbon solvent. Non-aromatic hydrocarbon solvents include, without limitation, heptane, hexane, cyclohexane, methylcyclohexane, heptane, and isooctane.

In some embodiments, the aprotic solvent is a polar aprotic solvent. Polar aprotic solvents include, without limitation, acetone, dichloromethane, cyclopentanone, methylisobutylketone, methylethylketone, EtOAc, isopropyl acetate, isobutyl acetate, glycerol diacetate, isoamyl acetate, tetrahydrofuran, dimethoxyethane, dioxane, N-methyl-2-pyrrolidone, CPME, 1,4-dioxane, THF, acetonitrile, DMSO, 2-MeTHF, Methyl tert-butyl ether (MTBE), 2,5-dimethylisosorbide, and chloroform.

In certain embodiments, the aprotic solvent is an ethereal solvent, e.g., tetrahydrofuran, dimethoxyethane, dioxane, CPME, 1,4-dioxane, or THF.

In certain embodiments, the aprotic solvent is acetonitrile or DMSO.

In some embodiments, the solvent is a protic solvent, e.g., a polar protic solvent, e.g., acetic acid.

Other suitable protic (polar) solvents include t-amyl alcohol, t-Butanol, n-propanol, ethanol, methanol, water, i-propanol, n-BuOH, t-Butanol, ethylene glycol, 1-Butanol, i-Amyl alcohol, 1-heptanol, 1-octanol, and 1-propanol.

In some embodiments, contacting the compound of formula (II) with the compound of formula (III) is carried out in the presence of a suitable mixture of two or more solvents, e.g., mixture of two solvents, e.g., a mixture of one or more (e.g., one) aromatic hydrocarbon solvents and one or more (e.g., one) ethereal solvents. For example, a mixture of toluene and dioxane (e.g., a 1:1 mixture of toluene and dioxane).

In some embodiments, contacting the compound of formula (II) with the compound of formula (III) is carried out at a temperature sufficient to produce the compound of formula (I). Those of skill in the art will readily be able to ascertain an appropriate temperature, using the methods described herein in combination with the knowledge in the art. Preferably, the reaction temperatures are above ambient temperature, that is, above 25° C., or above 35° C., or above. For example, suitable temperatures for conversion to a compound of formula (I) are temperatures that are at or below the reflux temperature of the reaction solvent. In other embodiments, a suitable temperature for preparing a compound of formula (I) is a temperature that is below the reflux temperature of the reaction solvent.

In certain embodiments, contacting the compound of formula (II) with the compound of formula (III) is carried out at a temperature of from about 80° C. to 110° C.; or from about 80° C. to 100° C. (e.g., 90° C. or 95° C.); or from about 90° C. to 110° C. (e.g., 100° C.).

In certain embodiments, contacting the compound of formula (II) with the compound of formula (III) is carried out at a temperature of from about 90° C. to 100° C. (e.g., 95° C.).

In certain embodiments, contacting the compound of formula (II) with the compound of formula (III) is carried out at a temperature of from about 80° C. to 100° C. (e.g., 85° C. to 95° C.; e.g., 90° C.).

In certain embodiments, contacting the compound of formula (II) with the compound of formula (III) is carried out at a temperature of from about 90° C. to 110° C. (e.g., 85° C. to 95° C.; e.g., 100° C.).

In certain embodiments, contacting the compound of formula (II) with the compound of formula (III) is carried out at a temperature of from about 20° C. to about 80° C. (e.g., 20° C.).

Those of ordinary skill in the art, using the methods described herein in combination with the knowledge in the art, will be readily able to ascertain an appropriate amount of time for the conversion of the compounds of formula (II) and formula (III) to the compounds of formula (I). For example, the conversion can be conducted until the conversion is substantially complete, as determined by HPLC. In some embodiments, the amount of time for substantial conversion to compounds of formula III is about 24 hours or less. In certain embodiments, the amount of time for substantial conversion is less than 24 hours, for example, about 20, 18, 16, 14, 12, 10, or about 8 hours.

In some embodiments, contacting the compound of formula (II) with the compound of formula (III) is carried out in the presence of one or more additives. Additives include, without limitation, Na₂SO₄, H₂O, H₂SO₄, acetic acid, formic acid, Bi(OTf)₃, PPh₃, NH₄OH, NH₄OAc, PPTS, PTSA, pyridine or any combination thereof.

In some embodiments, the contacting the compound of formula (II) with the compound of formula (III) is carried out in a sealed container filled with air.

In some embodiments, the contacting the compound of formula (II) with the compound of formula (III) is carried out in a sealed container filled with inert gas (e.g., nitrogen).

In some embodiments, the contacting the compound of formula (II) with the compound of formula (III) is carried out in an open container.

In some embodiments, the contacting the compound of formula (II) with the compound of formula (III) is carried out in an open container connected to an inert gas (e.g., nitrogen) manifold.

The method of any one of claims 1-48, wherein contacting the compound of formula (II) with the compound of formula (III) is carried out in the absence of an oxidizing agent; e.g., a peroxy acid, e.g., m-CPBA.

Examples of formula (I) compounds that can be prepared by the methods described herein include, but are not limited to, those described generically and specifically in PCT/US2021/051504, filed on Sep. 22, 2021; PCT/US2021/054191, filed on Oct. 8, 2021; and PCT/US2021/057348, filed on Oct. 8, 2021

### Compounds of Formula (II) and Preparation Thereof

In some embodiments, the methods further include contacting a compound of formula (IIa) with a compound of formula (IIb) to provide the compound of formula (II):

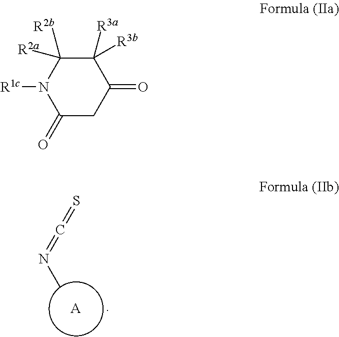

In some embodiments, the compound of formula (IIb) is prepared by contacting a chlorinating agent with a compound of formula (IId):

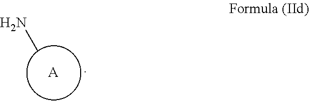

Chlorinating agents include, without limitation, thionyl chloride, methanesulfonyl Chloride, trichloromethanesulfonyl chloride, tert-butyl hypochlorite, dichloromethyl methyl ether, methoxyacetyl chloride, oxalyl chloride, cyanuric chloride, N-chlorosuccinimide, 1,3-Dichloro5,5-dimethylhydantoin, sodium dichloroisocyanurate, trichloroisocyanuric acid, chloramine T trihydrate, PCl₅, and POCl₃.

By way of example the compound of formula (IIb) can be prepared by contacting a compound of formula (IIc) with a compound of formula (IId):

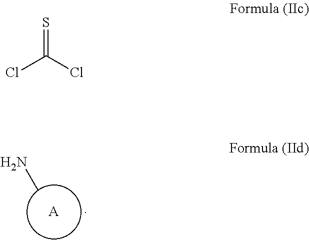

In some embodiments, Y in formula (II) is OH.

In some embodiments, Y in formula (II) is NH₂.

Ring A can be as defined anywhere herein.

Variables R^{1c}, R^{2a}, R^{2b}, R^{3a}, and R^{3b} can be as defined anywhere herein.

An exemplary formula (II) compound is:

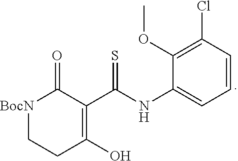 [SMILES: COc1c(Cl)cccc1NC(=S)C1=C(O)CCN(C(=O)OC(C)(C)C)C1=O]

### Compounds of Formula (III) and Preparation Thereof

In some embodiments, Z in formula (III) is —C(═O)H.

In some embodiments, Z in formula (III) is —CH(R)₂—. In certain embodiments, each R is halo (e.g., bromo). For example, Z can be —CH(Br)₂. In other embodiments, each R is alkoxy (e.g., OCH₃). For example, Z can be —CH(OCH₃)₂. In still other embodiments, one of R is OH, and the other R is SO₃⁻M⁺ (M+=Li, Na, K or NH₄⁺). For example, Z can be —CH(OCH₃)(SO₃⁻Na⁺).

In some embodiments of formula (III), X is X*. In certain embodiments, formula (III) is further substituted with a substituent reactive in Sonogashira coupling reactions, e.g., I, Br, Cl, F, triflate, tosylate, —C(O)Cl, and arylsulfonium triflate salts such as triarylsulfonium triflate salts, alkyl(diaryl)sulfonium triflate salts, and aryl(dialkyl)sulfonium triflate salts. In certain embodiments, the X* is halo, e.g., bromo.

In some embodiments of formula (III), X is X¹.

In certain embodiments, X¹ is

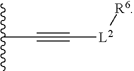

In certain embodiments, when formula (III) is substituted with

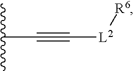

L² is a bond.

In certain embodiments, when formula (III) is substituted with

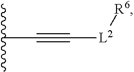

—R^{g2}—R^{W}.

In certain of the foregoing embodiments, —R⁶ is

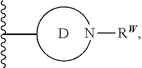

wherein Ring D is heterocyclylene including from 3-10 ring atoms, wherein from 0-2 ring atoms (in addition to the ring nitrogen atom bonded to R^{W}) are heteroatoms, each independently selected from the group consisting of N, N(H), N(R^{d}), O, and S(O)₀₋₂, and wherein the heterocyclylene is optionally substituted with from 1-3 substituents each independently selected from the group consisting of: oxo and —R^{c}; optionally wherein —R⁶ is a monocyclic heterocyclylene ring including from 3-10 ring atoms as defined above with a nitrogen atom bonded to R^{W}

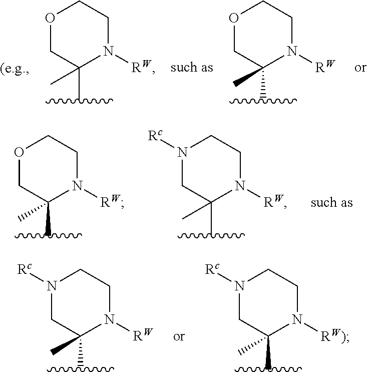

optionally wherein —R⁶ is a bicyclic heterocyclylene ring including from 3-10 ring atoms as defined above with a nitrogen atom bonded to R^{W}

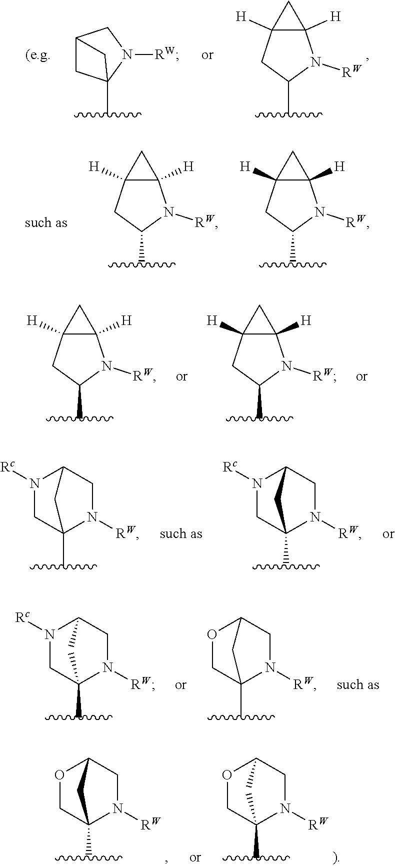

In certain of these embodiments, —R⁶ is

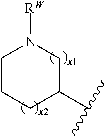

optionally substituted with from 1-2 R^{c}, wherein x1 and x2 are each independently 0, 1, or 2.

In certain embodiments, x1=0, and x2=0; or x1=0, and x2=1; or x1=0, and x2=2.

As non-limiting examples when R⁶ is

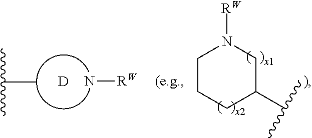

R⁶ can be selected from the group consisting of:

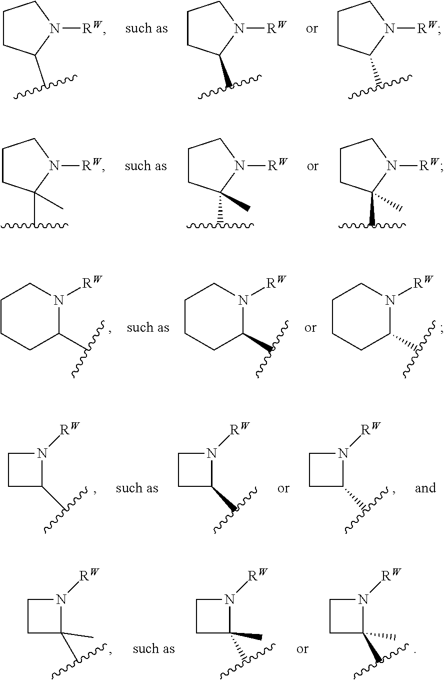

Ring C in formula (III) can be as defined anywhere herein.

Compounds of formula (III) can be prepared by conventional methods known to those of skill in the art and/or can be obtained commercially. An exemplary formula (III) compound is:

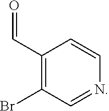

As the skilled person will appreciate, performing the methods described herein with formula (III) compounds, in which X is X* is expected to also produce formula (I) compounds, in which X is X*. Accordingly, the methods described herein further include converting the resultant formula (I) compounds, in which X is X* to formula (I) compounds, in which X is X¹. Reagents and conditions for converting the resultant formula (I) compounds, in which X is X* to formula (I) compounds, in which X is X¹ will be apparent to the skilled artisan and representative syntheses are provided in the Examples section.

Variables R^{1c}, R^{2a}, R^{2b}, R³, and R^{3b}In some embodiments, Y is —OH.

In some embodiments, R^{1c} is a protecting group.

Protecting groups include, without limitation, t-Butyloxycarbonyl(Boc), Benzyloxycarbonyl (Z), 9-Fluorenylmethoxycarbonyl (Fmoc), Allyloxycarbonyl (Alloc), Trityl (Trt), acetyl, benzyl, and p-Nitrobenzyloxycarbonyl (pNZ).

In certain embodiments, R^{1c} together with the nitrogen atom to which it is attached forms a carbamate. For example, R^{1c} can be a Boc group.

In certain of these embodiments, the methods further include removing the protecting group from the compound of formula (I), e.g., using conventional deprotection conditions know to those of skill in the art or by conducting the reaction to form the compound of formula (I) at an elevated temperature (e.g., 120° C.).

In some embodiments, R^{1c} is H.

In some embodiments, each of R^{2a}, R^{2b}, R^{3a}, and R^{3b} is independently selected from the group consisting of: H; halo; —OH; —C(O)OH or —C(O)NH₂; —CN; —R^{b}-L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}.

In certain of these embodiments, one of R^{2a}, R^{2b}, R^{3a}, and R^{3A} is independently selected from the group consisting of: halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; and the other of R^{2a}, R^{2b}, R^{3a}, and R^{3b} is H.

In some embodiments, two of variables R^{2a}, R^{2b}, R^{3a}, and R^{3b}, together with the Ring B ring atoms to which each is attached, form a fused saturated or unsaturated ring of 3-12 ring atoms.

In some embodiments, each of R^{2a}, R^{2b}, R^{3a}, and R^{3b} is H.

In some embodiments, each of the foregoing definitions of R^{1c}, R^{2a}, R^{2b}, R^{3a}, and R^{3b} apply to compounds of formula (II).

In some embodiments, each of the foregoing definitions of R^{1c}, R^{2a}, R^{2b}, R^{3a}, and R^{3b} apply to compounds of formula (I).

### Ring A

In some embodiments, ring A is C₆₋₁₀ aryl optionally substituted with from 1-4 R^{c}.

In certain embodiments, ring A is phenyl optionally substituted with from 1-4 R^{c}.

For example, ring A can be phenyl substituted with from 1-2 R^{c}.

In certain embodiments, Ring A is

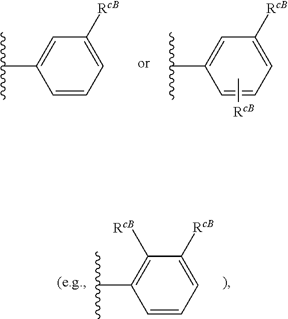

wherein each R^{cB} is an independently selected R^{c}.

In certain embodiments, each R^{cB} is independently selected from the group consisting of: -halo, such as —Cl and —F; —CN; C₁₋₄ alkoxy; C₁₋₄ haloalkoxy; C₁₋₃ alkyl; and C₁₋₃ alkyl substituted with from 1-6 independently selected halo.

In certain embodiments, Ring A is

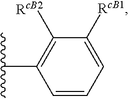

wherein R^{cB1} is R^{c}; and R^{cB2} is H or R^{c}.

In certain of these embodiments, R^{cB1} is halo (e.g., —F or —Cl (e.g., —F)).

In certain embodiments, R^{cB2} is C₁₋₄ alkoxy or C₁₋₄ haloalkoxy (e.g., C₁₋₄ alkoxy (e.g., methoxy)).

As non-limiting examples of the foregoing embodiments, Ring A can be

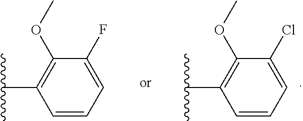

In some embodiments, each of the foregoing definitions of ring A apply to compounds of formula (II).

In some embodiments, each of the foregoing definitions of ring A apply to compounds of formula (I).

### Ring C

In some embodiments, Ring C is

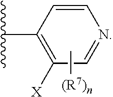

In some embodiments, Ring C is

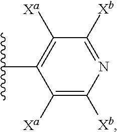

wherein:
each X^{b} is independently X, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅.

In some embodiments, Ring C is 2-pyridyl or 3-pyridyl, each optionally substituted with X and further optionally substituted with from 1-4 R^{c}.

In some embodiments, Ring C is 2-pyridonyl or 4-pyridonyl, each optionally substituted with X and further optionally substituted with from 1-4 R^{c}, wherein the ring nitrogen atom is optionally substituted with R^{d}.

In some embodiments, Ring C is heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c}.

In certain embodiments, Ring C is

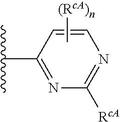

further optionally substituted with X, wherein each R^{cA} is an independently selected R^{c}; and n is 0, 1, or 2.

As a non-limiting example of the foregoing embodiments, Ring C can be

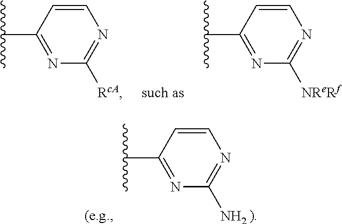

In certain foregoing embodiments, n is 0 and R^{cA} is C₁₋₁₀ alkyl optionally substituted with from 1-6 independently selected R^{a}, e.g., C₁₋₃ alkyl optionally substituted with from 1-3 independently selected halo.

As a non-limiting example, Ring C can be

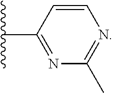 [SMILES: *c1ccnc(C)n1]

In some embodiments, Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c}.

In some embodiments, Ring C is bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is substituted with X and optionally substituted with from 1-4 substituents independently selected from the group consisting of oxo and R⁷.

In some embodiments, Ring C is bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷.

In some embodiments, Ring C is bicyclic C₅₋₁₀ cycloalkyl or C₅₋₁₀ cycloalkenyl, each of which is substituted with X and is optionally substituted with from 1-4 substituents independently selected from the group consisting of oxo and R⁷.

In some embodiments, Ring C is bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷.

In some embodiments, Ring C is heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is substituted with X and optionally substituted with from 1-4 substituents independently selected from the group consisting of oxo and R⁷.

In some embodiments, Ring C is heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷.

In some embodiments, Ring C is selected from the group consisting of:

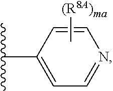

wherein
   ma is 0, 1, 2, or 3;
   R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A};
   R^{5A} is selected from hydroxy, halogen, cyano, C₁₋₄-alkyl, C₁₋₄-alkoxy, C₁₋₄ haloalkyl or C₁₋₄ haloalkoxy;
   R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{5A};
   R^{11A} is independently selected from C₁₋₄ alkyl, C₃₋₆ cycloalkyl, C₁₋₄ haloalkyl or C₃₋₆ halocycloalkyl;

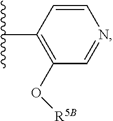

wherein
   R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{5A}; or
   R^{5B} is R^{6B}—CH₂—;
   R^{6B} is selected from

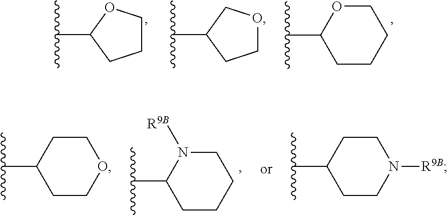

R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy;

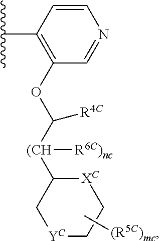

wherein
   R^{4C} is selected from hydrogen or methyl;
   R^{6C} is selected from hydrogen, C₁₋₃ alkyl, C₁₋₃ haloalkyl;
   nc is 0 or 1;
   X^{C} is NR^{7C} or O;
   Y^{C} is NR^{7C} or O;
   R^{7C} is methyl;
   R^{8C} is selected from methyl, 2,2,2-trifloethyl, or 2,2-difluoroethyl;
   R^{5C} is selected from hydrogen or methyl, wherein R^{5C} being attached to any carbon atom of the ring comprising X^{C} and Y^{C};
   mc is 0, 1, 2, or 3;

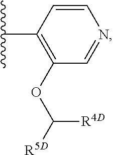

wherein
   R^{4D} is selected from hydrogen or methyl;
   R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R^{6D} and wherein each azetidinyl is substituted at the nitrogen with R^{8D}; OR
   R^{5D} is selected from the group consisting of

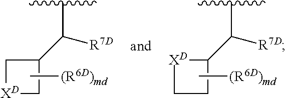

R^{6D} is selected from fluoro or C₁₋₃ alkyl;
md is selected from 0, 1, 2, or 3;
R^{7D} is selected from hydrogen, C₁₋₃ alkyl, or C₁₋₃ haloalkyl;
X^{D} is NR^{8D} of O;
R^{8D} is selected from C₁₋₃ alkyl or C₂₋₃ haloalkyl;

In some embodiments, Ring C is C₁₀ or C₁₄ aryl substituted with X and optionally substituted with from 1-4 R⁷.

In some embodiments, each of the foregoing definitions of ring A apply to compounds of formula (III).

In some embodiments, each of the foregoing definitions of ring A apply to compounds of formula (II).

In some embodiments, each of the foregoing definitions of ring A apply to compounds of formula (I).

The compounds, intermediates, and reagents disclosed herein can be prepared in a variety of ways in addition to those described herein, using, e.g., commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes and Examples provided below, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

Compounds described herein can be isolated/purified using conventional methods know to those skilled in the art, e.g., column chromatography, crystallization, HPLC (e.g., chiral HPLC).

### EXAMPLES

A number of embodiments of the invention have been described. Nevertheless, it will be understood that various modifications may be made without departing from the spirit and scope of the invention. Accordingly, other embodiments are within the scope of the following claims.

### Example 1. Synthesis of tert-butyl 2-(3-bromopyridin-4-yl)-3-((3-chloro-2-methoxyphenyl)amino)-4-oxo-1,4,6,7-tetrahydro-5H-pyrrolo[3,2-c]pyridine-5-carboxylate

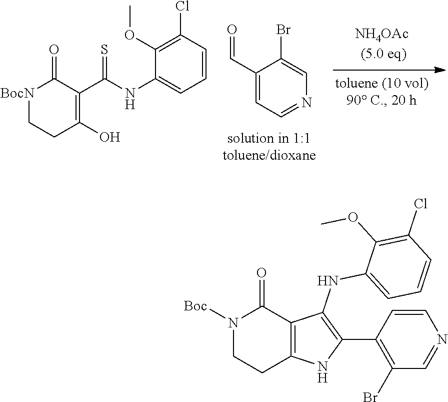

A suspension of 3-bromopyridine-4-carbaldehyde (3.38 g, 18.2 mmol, 1.5 equiv) in dioxane (37.5 mL)/dioxane (37.5 mL) was stirred under heating (in a 80° C. mantle) until the aldehyde had dissolved. The resulting solution was then added dropwise by syringe pump over 3.0 hours to a 250 mL round-bottom flask containing a 95° C. mixture of tert-butyl 3-[(3-chloro-2-methoxyphenyl)carbamothioyl]-2,4-dioxopiperidine-1-carboxylate (5.00 mg, 12.1 mmol, 1.0 equiv) and NH₄OAc (4.67 g, 60.6 mmol, 5.0 equiv) in toluene (50.0 mL) under an atmosphere of nitrogen. The mixture was then stirred for 21 hours at that temperature (total reaction time 24 h). The reaction mixture was quenched with saturated aqueous NaHCO₃ (100 mL) and diluted with MTBE (50 mL). The phases were separated and the aqueous layer extracted with MTBE (50 mL). The combined organic extracts were washed with saturated aqueous NaHCO₃ (50 mL) and brine (50 mL), dried over Na₂SO₄, filtered, and concentrated under reduced pressure. The residue was purified by silica gel column chromatography, eluted with n-heptane/EtOAc (3:1 to 0:1) to afford tert-butyl 2-(3-bromopyridin-4-yl)-3-((3-chloro-2-methoxyphenyl)amino)-4-oxo-1,4,6,7-tetrahydro-5H-pyrrolo[3,2-c]pyridine-5-carboxylate (3.03 g, 45.6% yield) as a yellow solid.

LCMS (ES, m/z): [M+H]⁺: 547/549;

¹H NMR (400 MHz, CDCl₃) δ 9.39 (s, 1H), 8.64 (s, 1H), 8.17 (d, J=5.4 Hz, 1H), 7.84 (s, 1H), 7.28 (d, J=5.3 Hz, 1H), 6.70 (dd, J=8.1, 1.4 Hz, 1H), 6.54 (t, J=8.1 Hz, 1H), 6.15 (dd, J=8.2, 1.5 Hz, 1H), 4.14 (t, J=6.3 Hz, 2H), 4.00 (s, 3H), 3.01 (t, J=6.4 Hz, 2H), 1.56 (s, 9H).

### Example 2. Synthesis of tert-butyl 5-((3-chloro-2-methoxyphenyl)carbamothioyl)-4-hydroxy-6-oxo-3,6-dihydropyridine-1(2H)-carboxylate

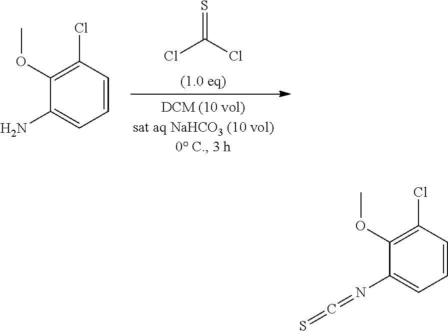

thiophosgene (6.08 mL, 79.3 mmol, 1.0 equiv) was added dropwise to a mixture of 3-chloro-2-methoxyaniline (12.5 g, 79.3 mmol, 1.0 equiv) in DCM (125 mL)/saturated aqueous NaHCO₃ (125 mL) at 0° C. and the mixture stirred at that temperature for 3 hours. The layers were separated and the organic layer washed with saturated aqueous NaHCO₃ and brine, dried over Na₂SO₄, filtered, and concentrated under reduced pressure to afford 1-chloro-3-isothiocyanato-2-methoxybenzene (16.2 g, quant. yield) as a brown oil.

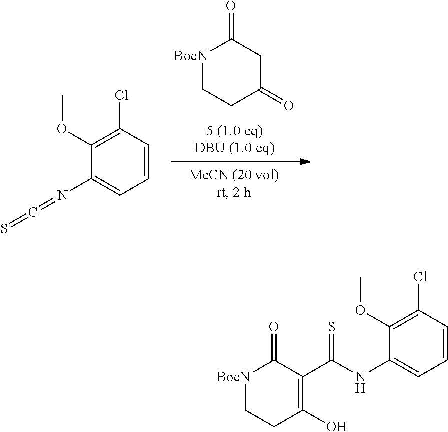 [SMILES: COc1c(Cl)cccc1N=C=S ; CC(C)(C)OC(=O)N1CCC(=O)CC1=O ; COc1c(Cl)cccc1NC(=S)C1=C(O)CCN(C(=O)OC(C)(C)C)C1=O]

DBU (17.2 mL, 115 mmol, 1.5 equiv) was added dropwise to a stirred suspension of tert-butyl 2,4-dioxopiperidine-1-carboxylate (16.4 g, 76.9 mmol, 1.0 equiv) and 1-chloro-3-isothiocyanato-2-methoxybenzene (16.2 g, 76.9 mmol, 1.0 equiv) in MeCN (375 mL) at room temperature and the reaction mixture stirred for 20 hours. The reaction mixture was diluted with ice water (100 mL) and HCl (1 M, 115 mL, 115 mmol, 1.5 equiv) was added to reach pH 6. The mixture was stirred for 15 minutes and then filtered to give 24 g of filter cake (86% purity). 1 g (×2) of the filter cake was taken and slurried in MeCN (4 mL) or EtOAc (2 mL) at room temperature overnight and then filtered to give material with >99% purity, with more material recovered from the MeCN slurry than EtOAc (0.70 g vs 0.45 g). The remaining cake (22 g) was slurried in 4 volumes (88 mL) of MeCN for 4 hours, filtered, and dried overnight in a vacuum oven at 50° C. to afford tert-butyl 3-[(3-chloro-2-methoxyphenyl)carbamothioyl]-2,4-dioxopiperidine-1-carboxylate (19.0 g, 59.8%) as a white solid. All of the filtrates were combined and purified by silica gel column chromatography, eluted with n-heptane/EtOAc (4:1 to 0:1) to afford tert-butyl 3-[(3-chloro-2-methoxyphenyl)carbamothioyl]-2,4-dioxopiperidine-1-carboxylate (4.54 g, 14.0%) as an off-white solid.

LCMS (ES, m/z): [M−H]⁻: 411, [M+H]⁺: 313 (des-Boc);

¹H NMR (400 MHz, CDCl₃) δ 13.67 (s, 1H), 7.78 (d, J=8.2 Hz, 1H), 7.32 (d, J=8.2 Hz, 1H), 7.09 (td, J=8.0, 2.5 Hz, 1H), 3.86 (m, 5H), 2.82 (t, J=6.9 Hz, 2H).

### Example 3. Synthesis of sodium (3-bromopyridin-4-yl)(hydroxy)methanesulfonate

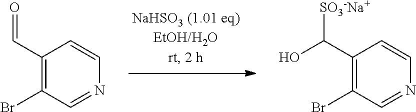

A solution of NaHSO₃ (565 mg, 5.43 mmol, 1.01 equiv) in water (1.8 mL) was added dropwise to a mixture of 3-bromopyridine-4-carbaldehyde (1.00 g, 5.38 mmol, 1 equiv) in EtOH (10 mL). The reaction mixture was stirred at room temperature for 2 hours and a solid had formed. EtOH (5 mL) was added and the mixture stirred for another 5 minutes and then filtered. The filter cake was washed with EtOH (2×2.5 mL) and dried in a vacuum oven overnight to afford sodium (3-bromopyridin-4-yl)(hydroxy)methanesulfonate (1.43 g, 91.7% yield) as a white solid.

LCMS (ESI, m/z): [M−Na]⁻ 266/268;

¹H NMR (400 MHz, DMSO-d₆) δ 8.59 (s, 1H), 8.44 (d, J=5.0 Hz, 1H), 7.64 (d, J=5.0 Hz, 1H), 6.38 (d, J=6.0 Hz, 1H), 5.29 (d, J=6.0 Hz, 1H).

### Example 4. Synthesis of tert-butyl 2-(3-bromopyridin-4-yl)-3-[(3-chloro-2-methoxyphenyl)amino]-4-oxo-1H,6H,7H-pyrrolo[3,2-c]pyridine-5-carboxylate

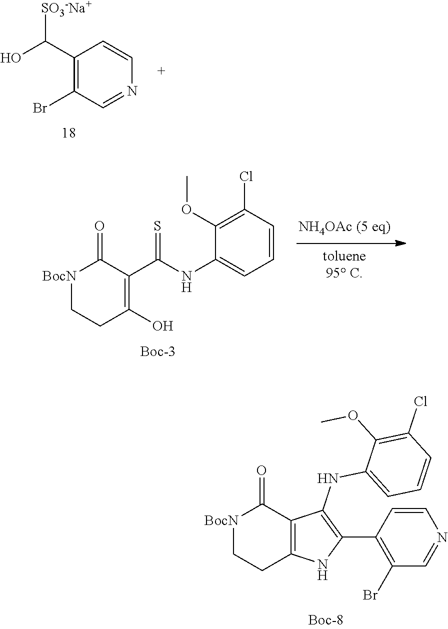

An oven-dried vial was charged with tert-butyl 3-[(3-chloro-2-methoxyphenyl)carbamothioyl]-4-hydroxy-2-oxo-5,6-dihydropyridine-1-carboxylate Boc-3 (50 mg, 0.121 mmol, 1 equiv) and ammonium acetate (46.7 mg, 0.61 mmol, 5 equiv.). Toluene (1 mL) was added and the formed suspension was heated at 95° C. Sodium (3-bromopyridin-4-yl)(hydroxy)methanesulfonate 18 (52.7 mg, 0.18 mmol, 1.5 equiv.) was added in five portions in a period of 2.5 h (0.3 equiv. every 30 minutes) and the reaction mixture was then stirred overnight (16-18 h) at 95° C. An aliquot was then taken and analyzed by LCMS and HPLC to give the following results:

LCMS: 44% Boc-8, 22% Boc-3

HPLC (toluene peak deleted): 60% Boc-8, 31% Boc-3

### Example 5. Representative Procedure 1 (GP1) for the Synthesis of Compounds of Formula (II)

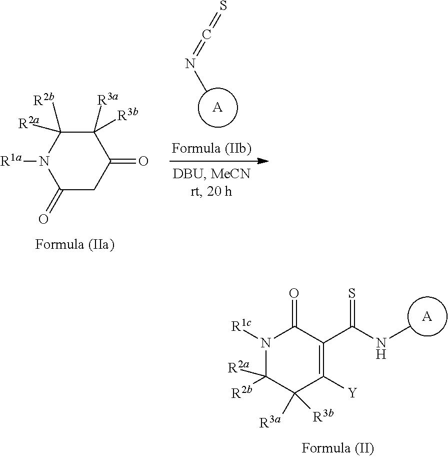

DBU (1.50 eq) was added to a stirred suspension of β-ketoamide (formula (IIa)) (1.00 eq) and isothiocyanate (formula (IIb)) (1.00 eq) in anhydrous acetonitirile (0.470 m) under N₂ atmosphere and the resulting mixture was stirred at ambient temperature for 20 h. The reaction was quenched with HCl (1.00 m) to pH 6, diluted with water and then extracted with EtOAc or DCM (3×50 mL). The combined organic extracts were washed with saturated aqueous NaHCO₃ and brine, dried over MgSO₄, filtered, and concentrated under reduced pressure. The crude residue was purified by silica gel chromatography to provide the compound (formula (II)).

### Example 6. Representative Procedure 2 (GP2) for the Synthesis of Compounds of Formula (I)

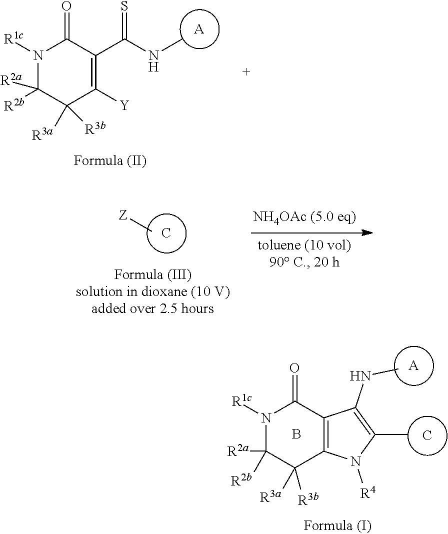

An oven-dried vial was charged with the compound of formula (II) (1.00 eq) and NH₄OAc (5.00 eq), and was evacuated and backfilled with N₂ three times. Anhydrous toluene (10.0 vol) was added, and the reaction was heated to 90° C. A premade solution of compound of formula (III) (1.50 eq) in anhydrous 1,4-dioxane (10.0 vol) was then added dropwise over 2.5 h with a syringe pump and the resulting mixture was stirred for 18 h at 90° C. The reaction was cooled to ambient temperature and directly concentrated under reduced pressure. The crude residue was purified by silica gel chromatography to provide the compound of formula (I).

### Example 7. GP2 Reaction Conditions

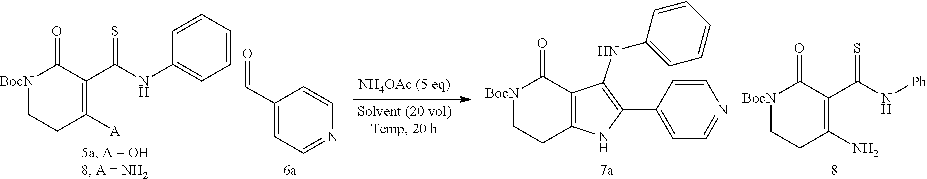

A.Starting Material: enamine 8^{a}

| Entry | Solvent | T/º C | HPLC area % (254 nm) |
|---|---|---|---|
| 1 | EtOH | 70 | 85% 8 |
| 2 | toluene | 70 | 91% 8/toluene |
| 3 | toluene | 100 | 4% des(Boc) 8, 81% 8/toluene |

B. Starting Material: enol 5ab

| | | | |
|---|---|---|---|
| Entry | Solvent | T /ºC | HPLC area % (254 nm)^{b} |
| 1 | EtOH | 70 | 37% 7a, 27% 8, 0% 5a |
| 2 | 1,4-dioxane | 70 | 60% 7a, 4% 8, 10% 5a |
| 3 | toluene | 70 | 17% 7a, 74% 5a |
| 4 | 1,4-dioxane | 100 | 59% 7a, 4% 8 |
| 5 | toluene | 100 | 35% 7a, 5% 8, 23% 5a |
| 6 | 1:1 toluene/1,4-dioxane | 70 | 56% 7a, 2% 8, 23% 5a |
| 7 | 1:1 toluene/1,4-dioxane | 100 | 66% 7a, 5% 8 |
| 8 | 2-MeTHF | 80 (reflux) | 42% 7a, 4% 8, 34% 5a |
| 9 | CPME | 100 | 41% 3, 9% 8 |
| 10 | 1:1 toluene/2-MeTHF | 100 | 59% 7a, 5% 8, 0% 5a |
| 11 | 1:1 toluene/CPME | 100 | 44% 7a, 7% 8, 0% 5a |

C. Variation of aldehyde equivalents^{c}

| | | |
|---|---|---|
| Entry | 6a eq | HPLC area % (254 nm)^{b} |
| 1 | 1.0 | 60% 7a, 4% 8, 10% 5a |
| 2 | 1.3 | 67% 7A, 5% 8, 8% 5a |
| 3 | 1.5 | 70% 7A, 5% 8, 6% 5a (58%) |
| 4 | 2.0 | 65% 7A, 3% 8, 4% 5a |

D. Variation of concentration, temperature, ammonium source, and additives^{d}

| | | | | | |
|---|---|---|---|---|---|
| | | | | Additive | |
| Entry | Solvent (vol) | T/° C. | NH₃ source (eq) | (eq) | HPLC area % (254 nm)^{b} |
| 1 | 1,4-dioxanc (20) | 70 | NH₄OAc (5.0) | — | 70% 7a, 5% 8, 6% 5a (58%) |
| 2 | 1,4-dioxane (10) | 70 | NH₄OAc (5.0) | — | 64% 7a, 4% 8, 10% 5a |
| 3 | 1,4-dioxane (40) | 70 | NH₄OAc (5.0) | | 64% 7a, 4% 8, 7% 5a |
| 4 | 1,4-dioxane (20) | 50 | NH₄OAc (5.0) | — | 59% 7a, 0% 8, 24% 5a |
| 5 | 1,4-dioxane (20) | 60 | NH₄OAc (5.0) | — | 59% 7a, 2% 8, 24% 5a |
| 6 | 1,4-dioxane (20) | 80 | NH₄OAc (5.0) | — | 68% 7a, 5% 8 |
| 7 | 1,4-dioxane (20) | 90 | NH₄OAc (5.0) | — | 64% 7a, 5% 8 |
| 8 | 1,4-dioxane (20) | 100 | NH₄OAc (5.0) | — | 59% 7a, 4% 8 |
| 9 | 1,4-dioxane (20) | 70 | (NH₄)2CO₃ (5.0) | — | 33% 7a, 1%8, 51% 5a |
| 10 | 1,4-dioxane (20) | 70 | NH₄C1 (5.0) | — | 98% 5a |
| 11 | 1,4-dioxane (20) | 70 | NH₄CO₂H (5.0) | — | 54% 7a, 25% 5a |
| 12 | 1,4-dioxane (20) | 70 | NH₄OAc (3.0) | — | 62% 7a, 2% 8, 12% 5a |
| 13 | 1,4-dioxane (20) | 70 | NH₄OAc (10.0) | — | 65% 7a, 3% 8, 10% 5a |
| 14 | 1,4-dioxane (20) | 70 | NH₄OAc (5.0) | H₂O (1.5) | 63% 7a, 3% 8, 5% 5a |
| 15 | 1,4-dioxane (20) | 70 | NH₄OAc (5.0) | AcOH (1.5) | 62% 7a, 3% 8, 6% 5a |
| 16 | 1,4-dioxane (20) | 70 | NH₄OAc (5.0) | FeCl₃ (0.5) | 25% 7a, 3% 8, 17% 5a |
| 17 | 1,4-dioxane (20) | 70 | NH₄OAc (5.0) | FeCl₃ (0.1) | 33% 7a, 3% 8, 14% 5a |

E. Variation of aldehyde addition and solvent mixturese

| | | | | |
|---|---|---|---|---|
| Entry | Solvent (vol) | T/° C. | Aldehyde | HPLC area % (254 nm)^{b} |
| 1 | 1,4-dioxane (20) | 70 | 1.5 eq at reaction onset | 70% 7a, 5% 8, 6% 5a (58%) |
| 2 | 1,4-dioxane (20) | 70 | 1.0 eq at reaction onset | 65% 7a, 2% 8, 3% 5a |
| | | | +0.5 eq after 2 h | |
| 3 | 1,4-dioxane (10) | 70 | 1.5 eq in 1,4-dioxane (10 vol), | 71% 7a, 4% 8, 5% 5a (60%) |
| | | | added over 2.5 h | |
| 4 | 1,4-dioxane (10) | 70 | 2.0 eq in 1,4-dioxane (10 vol), | 58% 7a, 3% 8 |
| | | | added over 2.5 h | |
| 5 | 1:1 toluene/1,4- | 90 | 1.5 eq at reaction onset | 69% 7a, 1% 5a |
| | dioxane (20) | | | |
| 6 | toluene (10) | 70 | 1.5 eq in 1,4-dioxane (10 vol), | 64% 7a, 10% 8, 21% 5a |
| | | | added over 2.5 h | |
| 7 | toluene (10) | 70 | 1.5 eq in 1: 1 toluene/1,4-dioxane | 63% 7a, 2% 8, 20% 5a |
| | | | (10 vol), added over 2.5 h | |
| 8 | toluene (10) | 90 | 1.5 eq in 1,4-dioxane (10 vol), | 80% 7a, 3% 8 (70%) |
| | | | added over 2.5 h | |
| 9 | toluene (10) | 90 | 1.5 eq in 2-MeTHF (10 vol), added | 71% 7a, 4% 8, 5% 5a (62%) |
| | | | over 2.5 h | |
| 10 | toluene (10) | 90 | 1.5 eq in 1: 1 toluene/1,4-dioxane | 72% 7a, 3% 8, 12% 5a |
| | | | (10 vol), added over 2.5 h | |
| 11 | toluene (10) | 90 | 1.5 eq in 1: 1 toluene/2-MeTHF | 73% 7a, 2% 8, 7% 5a (67%) |
| | | | (10 vol), added over 2.5 h | |

^{a}Reaction conditions: 8 (0.144 mmol), 6a (0.144 mmol), NH₄OAc (0.720 mmol), Solvent (1.0 mL), Temperature, 20 h.
^{b}Reaction conditions: 5a (0.144 mmol), 6a (0.144 mmol), NH₄OAc (0.720 mmol), Solvent (1.0 mL), Temperature, 20 h.
^{c}Reaction conditions: 5a (0.144 mmol), 6a, NH4OAc (0.720 mmol), 1,4-dioxane (1.0 mL), 70 °C, 20 h.
^{b}Isolated yield in brackets.
^{d}Reaction conditions: 5a (0.144 mmol), 6a (0.216 mmol), NH₃ source, Solvent, Temperature, 20 h.
^{b}Isolated yield in brackets.
^{e}Reaction conditions: 5a (0.144 mmol), 6a, NH4OAc (0.720 mmol), Solvent, Temperature, 20 h.
^{b}Isolated yield

### Example 8. Reaction Condition Optimization

Reactions of enol 5a, NH-4OAc, and 4-pyridinecarboxaldehyde (6a) under various conditions were investigated (Table 1). When heated together in EtOH at 70° C. overnight, the starting material was consumed and 3700 of the desired product 7a was detected by HPLC analysis (entry 1). Enamine 8 was identified as a major side-product (270%). To investigate whether this enamine is productive, 8 was independently synthesized and subjected to the reaction conditions with no conversion to 7a observed.¹¹ While not wishing to be bound by theory, it is believed that the reaction mechanism proceeds through initial condensation of ammonia with the aldehyde. Utilization of 1,4-dioxane resulted in increased conversion to the desired product 7a (60%) and reduction of enamine 8 (4%); however, when the reaction was performed in toluene 74% of the starting material was left unconsumed (entries 2 and 3). Other ethereal solvents, such as THF, CPME, and TBME were also productive.¹¹ Increasing the equivalents of aldehyde from 1.0 to 1.5 was beneficial (entry 4) and gave 70% of pyrrole 7a (isolated in 58% yield); however, increasing the equivalents further provided no apparentl advantage (entry 5). Use of (NH₄)₂CO₃ resulted in a significant reduction in conversion, whereas NH₄Cl provided no detectable levels of product (entries 6 and 7).¹ The reaction was relatively insensitive to the equivalents of NH₄OAc but adding >5 equivalents did not appear to be beneficial (entries 8 and 9). Conducting the reaction at 90° C. resulted in complete conversion of the starting material and 64% of pyrrole 7a was detected (entry 11), whereas decreasing the temperature to 50° C. led to a significant reduction in product formation (entry 11). It was still observed that when the reaction was performed in toluene (entry 3), formation of enamine 8 was also suppressed. In a mixed solvent system 69% of the product 7a was observed (entry 12), matching the conversion observed in 1,4-dioxane. Additionally, full consumption of the starting material was observed and formation of enamine 8 was minimized.

TABLE 1^{a}

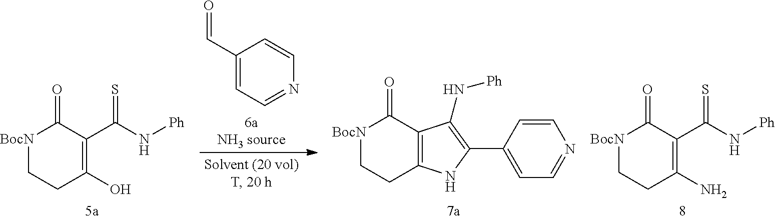 [SMILES: CC(C)(C)OC(=O)N1CCC(O)=C(C(=S)Nc2ccccc2)C1=O ; O=Cc1ccncc1 ; CC(C)(C)OC(=O)N1CCc2[nH]c(-c3ccncc3)c(Nc3ccccc3)c2C1=O ; CC(C)(C)OC(=O)N1CCC(N)=C(C(=S)Nc2ccccc2)C1=O]

| | | | 6 | | HPLC area % |
|---|---|---|---|---|---|
| Entry | Solvent | T/° C. | eq | NH₃ source (eq) | 7/8/5^{b} |
| 1 | ethanol | 70 | 1.0 | NH₄OAc (5) | 37/27/0 |
| 2 | 1,4-dioxane | 70 | 1.0 | NH₄OAc (5) | 60/4/10 |
| 3 | toluene | 70 | 1.0 | NH₄OAc (5) | 17/0/74 |
| 4 | 1,4-dioxane | 70 | 1.5 | NH₄OAc (5) | 70/5/6 |
| | | | | | (58%) |
| 5 | 1,4-dioxane | 70 | 2.0 | NH₄OAc (5) | 65/3/4 |
| 6 | 1,4-dioxane | 70 | 1.5 | NH₄Cl (5) | 0/0/98 |
| 7 | 1,4-dioxane | 70 | 1.5 | (NH₄)₂CO₃ (5) | 33/1/51 |
| 8 | 1,4-dioxane | 70 | 1.5 | NH₄OAc (3) | 62/2/12 |
| 9 | 1,4-dioxane | 70 | 1.5 | NH₄OAc (10) | 65/3/10 |
| 10 | 1,4-dioxane | 90 | 1.5 | NH₄OAc (5) | 64/5/0 |
| 11 | 1,4-dioxane | 50 | 1.5 | NH₄OAc (5) | 59/0/24 |
| 12 | toluene/1,4- | 90 | 1.5 | NH₄OAc (5) | 69/0/1 |
| | dioxane | | | | |
| 13 | 1,4- | 70 | 1.5 | NH₄OAc (5) | 71/4/5 |
| | dioxane^{c} | | | | (61%) |
| 14 | toluene^{c} | 90 | 1.5 | NH₄OAc (5) | 80/3/0 |
| | | | | | (70%) |

^{a}5a (0.144 mmol), 6a, NH₃ source, Solvent (20 vol), T °C, 20 h.
^{b}Isolated yield in brackets.
^{c}6a was added as a solution in 1,4-dioxane (10 vol) over 2.5 h to 5a and NH₄OAc in Solvent (10 vol).

### Example 9. Synthesis of Compounds 9 and 10

### tert-Butyl 5-oxo-2-phenyl-4-(phenylamino)-7,8-dihydro-2H-pyrido[4,3-d][1,3]thiazine-6(5H)-carboxylate, 9

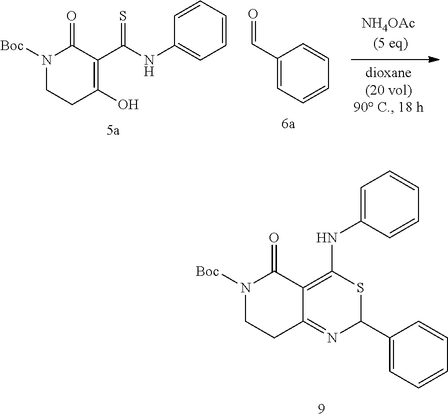

A stirred solution of 5a (500 mg, 1.44 mmol, 1.00 equiv), benzaldehyde (220 μL, 2.15 mmol, 1.50 eq), and NH₄OAc (553 mg, 7.18 mmol, 5.00 eq) in 1,4-dioxane (10.0 mL) was heated to 70° C. overnight. The reaction mixture was diluted with water (50 mL) and filtered. The precipitate was purified by silica gel chromatography using a 40 g SiO₂ cartridge and a linear gradient of 0-50% EtOAc in heptane over 18 CV, to afford 9 (140 mg, 19%, 85% purity) as a yellow solid.

¹H NMR (400 MHz, Chloroform-d) δ 13.33 (s, 1H), 7.49-7.42 (m, 2H), 7.28 (dd, J=8.2, 6.5 Hz, 4H), 7.25-7.16 (m, 2H), 7.15-7.05 (m, 2H), 5.55 (d, J=1.8 Hz, 1H), 3.97 (dt, J=12.9, 5.3 Hz, 1H), 3.62 (ddd, J=12.9, 10.9, 3.8 Hz, 1H), 2.85 (dddd, J=15.9, 11.0, 4.9, 1.9 Hz, 1H), 2.73 (ddd, J=15.0, 5.7, 3.8 Hz, 1H), 1.50 (s, 9H); ¹³C NMR (101 MHz, Chloroform-d) δ 167.8, 166.8, 165.4, 152.0, 138.9, 136.8, 129.0, 128.8, 128.8, 127.9, 127.5, 125.4, 94.8, 83.1, 64.1, 42.8, 33.7, 28.1;

HRMS (ESI) calculated for C₂₁H₂₅N₃O₃S⁺ [M+H]⁺ 436.1695; found m/z 436.1697.

### Example 10. Conversion of 9 to Pyrrole 7ac

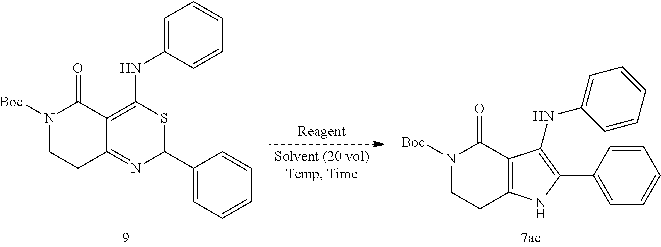 [SMILES: CC(C)(C)OC(=O)N1CCC2=NC(c3ccccc3)SC(Nc3ccccc3)=C2C1=O ; CC(C)(C)OC(=O)N1CCc2[nH]c(-c3ccccc3)c(Nc3ccccc3)c2C1=O]

| Entry | Reagent (eq) | Solvent | Temp | Time | Result (LCMS PDA area %) |
|---|---|---|---|---|---|
| 1 | BiOTf (0.5) | 1,4-dioxane | 100° C. | 18 h | Boc deprotection only |
| 2 | DBU (1) | 1,4-dioxane | 100° C. | 18 h | 9 consumed, trace 7ac |
| | | | | | detected |
| 3 | NEt₃ (1) | 1,4-dioxane | 100° C. | 18 h | 30% 9, trace 7ac detected |
| 4 | NEt₃ (5) | 1,4-dioxanc | 100° C. | 18 h | 21% 9, tracc 7ac detected |
| 5 | NEt₃ (5) | toluene | 100° C. | 4 h | 14% 9, trace 7ac detected |
| 6 | NEt₃ (5) | CPME | 100° C. | 4 h | 30% 9, trace 7ac detected |
| 7 | KO^{t}Bu (5) | 1,4-dioxane | 100° C. | 18 h | 9 consumed, no 7ac detected |
| 8 | PPh₃ (1.5) | 1,4-dioxane | 100° C. | 18 h | No reaction |
| 9 | TFA (2) | 1,4-dioxane | 100° C. | 4 h | Boc deprotection only |
| 10 | CSA (2) | 1,4-dioxane | 100° C. | 4 h | Boc deprotection only |
| 11 | PTSA•H20 (2) | 1,4-dioxane | 100° C. | 4 h | Boc deprotection only |
| 12 | AcOH (5) | 1,4-dioxane | 100° C. | 18 h | <5% 7ac detected |
| 13 | AcOH (5) | toluene | 100° C. | 4 h | trace 7ac detected |
| 14 | AcOH (5) | 1:1 toluene/2- | 100° C. | 4 h | trace 7ac detected |
| | | MeTHF | | | |
| 15 | AcOH (5) | CPME | 100° C. | 4 h | trace 7ac detected |
| 16 | BzOH (5) | 1,4-dioxane | 100° C. | 4 h | 9 consumed, trace 7ac |
| | | | | | detected |
| 17 | PPTS (5) | 1,4-dioxane | 100° C. | 4 h | 21% Boc deprotection |
| 18 | [Ru(phen)₃] Cl₂ | MeCN | rt | 18 h | No reaction |
| | (0.1), air, blue LED | | | | |
| 19 | [Ru(phen)₃]Cl₂ | DMF | rt | 18 h | No reaction |
| | (0.1), air, blue LED | | | | |
| 20 | [Ru(phen)₃]Cl₂ | DMF | rt | 18 h | No reaction |
| | (0.1), | | | | |
| | benzophenone (1), | | | | |
| | blue LED | | | | |
| 21 | Rose Bengal (0.1), | DMF | rt | 18 h | No reaction |
| | air, blue LED | | | | |
| 22 | Rose Bengal (0.1), | DMF | rt | 18 h | No reaction |
| | benzophenonc (1), | | | | |
| | blue LED | | | | |
| 23 | MeI (2.8) | DMF | rt-80° C. | 18 h | New peak with MW of |
| | | | | | methylation, no P detected |
| 24 | Selectfluor (1.2) | DMF | rt | 2 h | 9% 9, 10% lactam oxidation |
| 25 | NFSI | DMF | rt | 2 h | 3% 9, 8% lactam oxidation |
| 26 | I₂ (1.2) | DMF | rt | 2 h | 16% 9, no 7ac detected |
| 27 | PhI(OAc)₂ (1.2) | DMF | rt | 2 h | 11% 9, no 7ac detected |
| 28 | I₂ (1.2) | EtOH | rt | 2 h | 9% 9, no 7ac detected |
| 29 | Selectfluor (1.2) | THF | rt | 2 h | 27%9, 11% lactam oxidation, |
| | | | | | trace 7ac detected |
| 30 | Selectfluor (1.2) | MeCN | rt | 2 h | 23% 9, 26% lactam oxidation |
| 31 | Selectfluor (1.2) | EtOH | rt | 2 h | 24% 9, no 7ac detected |

### Example 11. Synthesis of tert-Butyl 4-hydroxy-6-oxo-5-(phenylcarbamothioyl)-3,6-dihydropyridine-1(2H)-carboxylate, 5a¹

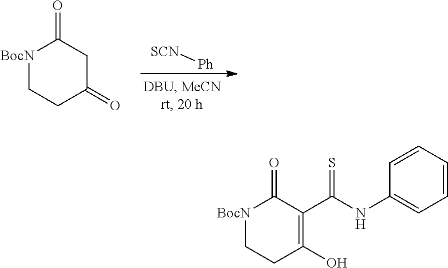

Synthesised according to GP1 using tert-butyl 2,4-dioxopiperidine-1-carboxylate (2.00 g, 9.38 mmol) and isothiocyanatobenzene (1.12 mL, 9.38 mmol). Purified by silica gel chromatography using a 40 g SiO₂ cartridge and a linear gradient of 0-50% EtOAc in heptane over 15 CV to give 5a (3.13 g, 96%) as a white solid.

¹H NMR (400 MHz, DMSO-d₆) δ 15.31 (s, 1H), 12.84 (s, 1H), 7.57 (d, J=7.9 Hz, 2H), 7.44 (t, J=7.7 Hz, 2H), 7.31 (t, J=7.4 Hz, 1H), 3.77 (t, J=6.5 Hz, 2H), 2.81 (t, J=6.4 Hz, 2H), 1.47 (s, 9H);

¹³C NMR (400 MHz, DMSO-d₆) δ 189.5, 181.7, 166.1, 152.4, 138.4, 129.3, 127.4, 125.2, 105.7, 82.8, 41.0, 30.8, 28.1;

HRMS (ESI) calculated for C₁₇H₂₁N₂O₄S⁺ [M+H]⁺ 349.1222; found m/z 349.1223.

The ¹H NMR data matched that of the literature.

### Example 12. Synthesis of tert-Butyl 4-amino-6-oxo-5-(phenylcarbamothioyl)-3,6-dihydropyridine-1(2H)-carboxylate, 8

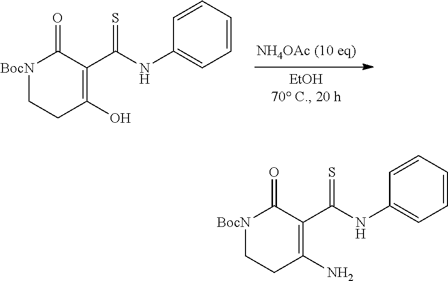 [SMILES: CC(C)(C)OC(=O)N1CCC(O)=C(C(=S)Nc2ccccc2)C1=O ; CC(C)(C)OC(=O)N1CCC(N)=C(C(=S)Nc2ccccc2)C1=O]

A mixture of 5a (1.00 g, 2.87 mmol, 1.00 eq) and NH₄OAc (2.21 g, 28.7 mmol, 10.0 eq) in EtOH (12 mL) was heated to 70° C. overnight. The reaction mixture was concentrated to ˜4 mL and then quenched with saturated aqueous NaHCO₃. The mixture was extracted with DCM (3×20 mL) and the combined organic extracts were dried over MgSO₄, filtered, and concentrated under reduced pressure. The residue was purified by silica gel chromatography, using a 40 g SiO₂ cartridge and a linear gradient of 5-100% EtOAc in heptane over 15 CV, to afford 8 (576 mg, 58%) as a light yellow solid.

¹H NMR (400 MHz, DMSO-d₆) δ 13.18 (s, 1H), 11.30 (s, 1H), 8.74 (s, 1H), 7.51 (d, J=7.9 Hz, 2H), 7.38 (t, J=7.8 Hz, 2H), 7.22 (t, J=7.4 Hz, 1H), 3.67 (t, J=6.3 Hz, 2H), 2.76 (t, J=6.2 Hz, 2H), 1.46 (s, 9H);

¹³C NMR (101 MHz, DMSO-d₆) δ 190.8, 168.0, 166.8, 152.8, 139.7, 128.9, 126, 125.6, 98.7, 82.1, 41.0, 31.3, 28.2;

HRMS (ESI) calculated for C₁₇H₂₀N₃O₃S⁻ [M−H]⁻ 346.1225; found m/z 346.1227.

### Example 13. Synthesis of tert-Butyl 5-((2-chlorophenyl)carbamothioyl)-4-hydroxy-6-oxo-3,6-dihydropyridine-1(2H)-carboxylate, 5r

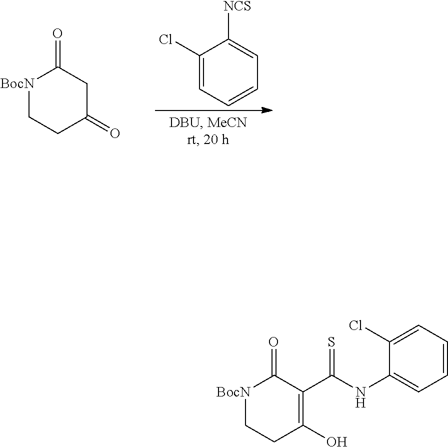

Synthesised according to GP1 using tert-butyl 2,4-dioxopiperidine-1-carboxylate (300 mg, 1.41 mmol) and 1-chloro-2-isothiocyanatobenzene (239 mg, 1.41 mmol). Purified by silica gel chromatography using a 40 g SiO₂ cartridge and a linear gradient of 0-25% EtOAc in heptane over 15 CV to give 5r (379 mg, 70%) as a white solid.

¹H NMR (400 MHz, Chloroform-d) δ 13.50 (s, 1H), 7.53 (d, J=7.7 Hz, 1H), 7.49 (d, J=7.7 Hz, 1H), 7.35-7.27 (m, 2H), 3.87 (t, J=6.6 Hz, 2H), 2.83 (t, J=6.6 Hz, 2H), 1.56 (s, 9H);

¹³C NMR (101 MHz, Chloroform-d) δ 191.1, 186.7, 167.9, 152.4, 135.6, 131.3, 130.6, 129.3, 129.3, 127.5, 102.5, 84.3, 40.8, 32.2, 28.5; HRMS (ESI) calculated for C₁₇H₁₉ClN₂O₃SNa⁺ [M+Na]⁺ 405.0652; found m/z 405.0651.

### Example 14. Synthesis of tert-Butyl 5-((3-chlorophenyl)carbamothioyl)-4-hydroxy-6-oxo-3,6-dihydropyridine-1(2H)-carboxylate, 5s

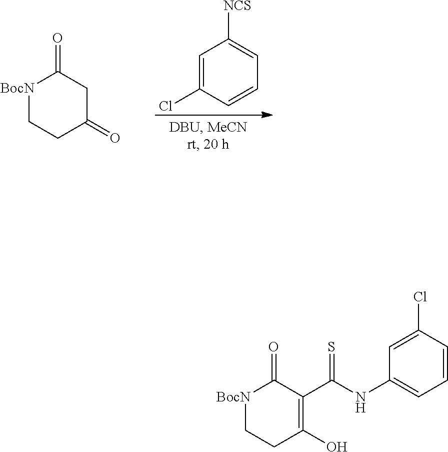

Synthesised according to GP1 using tert-butyl 2,4-dioxopiperidine-1-carboxylate (300 mg, 1.41 mmol) and 1-chloro-3-isothiocyanatobenzene (239 mg, 1.41 mmol). Purified by silica gel chromatography using a 40 g SiO₂ cartridge and a linear gradient of 0-30% EtOAc in heptane over 15 CV to give 5s (360 mg, 67%) as an off-white solid.

¹H NMR (400 MHz, Chloroform-d) δ 13.57 (s, 1H), 7.43 (s, 1H), 7.32-7.24 (m, 1H), 7.22-7.19 (m, 2H), 3.78 (t, J=6.6 Hz, 2H), 2.75 (t, J=6.6 Hz, 2H), 1.49 (s, 9H);

¹³C NMR (101 MHz, Chloroform-d) δ 189.7, 186.4, 167.8, 152.0, 138.8, 134.6, 130.0, 127.6, 126.1, 124.1, 102.1, 84.1, 40.5, 31.9, 28.2.

HRMS (ESI) calculated for C₁₇H₂₀ClN₂O₃S⁺ [M+H]⁺ 383.0832; found m/z 383.0829.

### Example 15. Synthesis of tert-Butyl 5-((4-chlorophenyl)carbamothioyl)-4-hydroxy-6-oxo-3,6-dihydropyridine-1(2H)-carboxylate, 5t

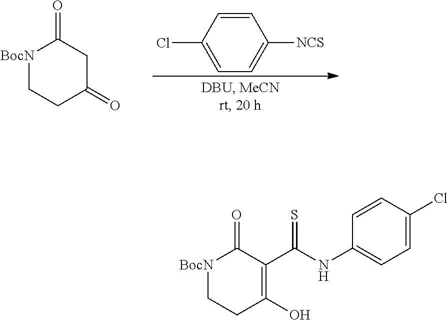

Synthesised according to GP1 using tert-butyl 2,4-dioxopiperidine-1-carboxylate (300 mg, 1.41 mmol) and 1-chloro-4-isothiocyanatobenzene (239 mg, 1.41 mmol). Purified by silica gel chromatography using a 40 g SiO₂ cartridge and a linear gradient of 0-35% EtOAc in heptane over 15 CV to give 5t (343 mg, 64%) as a white solid.

¹H NMR (400 MHz, Chloroform-d) δ 13.60 (s, 1H), 7.38 (s, 4H), 3.85 (t, J=6.6 Hz, 2H), 2.81 (t, J=6.6 Hz, 2H), 1.56 (s, 9H);

¹³C NMR (101 MHz, Chloroform-d) δ 189.9, 186.6, 168.1, 152.2, 136.5, 133.3, 129.6, 127.6, 102.4, 84.4, 40.8, 32.2, 28.5;

HRMS (ESI) calculated for C₁₇H₂OClN₂O₃S⁺ [M+H]⁺ 383.0832; found m/z 383.0826.

### Example 16. Synthesis of tert-Butyl 4-hydroxy-5-((2-methoxyphenyl)carbamothioyl)-6-oxo-3,6-dihydropyridine-1(2H)-carboxylate, 5u²

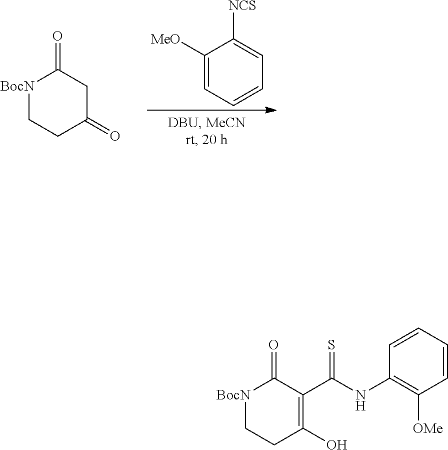

Synthesised according to GP1 using tert-butyl 2,4-dioxopiperidine-1-carboxylate (300 mg, 1.41 mmol) and 1-isothiocyanato-2-methoxybenzene (190 L, 1.41 mmol). Purified by silica gel chromatography using a 40 g SiO₂ cartridge and a linear gradient of 0-35% EtOAc in heptane over 15 CV to give 5u (396 mg, 74%) as a white solid.

¹H NMR (400 MHz, Chloroform-d) δ 13.33 (s, 1H), 7.66 (d, 1H), 7.18 (t, J=7.8, 1.6 Hz, 1H), 6.91-6.86 (m, 2H), 3.78-3.71 (m, 5H), 2.69 (t, J=6.6 Hz, 2H), 1.47 (s, 9H);

¹³C NMR (101 MHz, Chloroform-d) δ 188.9, 185.7, 167.5, 153.4, 152.2, 128.5, 127.0, 126.6, 120.2, 111.6, 102.4, 83.7, 56.1, 40.5, 31.9, 28.3; HRMS (ESI) calculated for C₁₈H₂₁N₂O₅S⁻ [M−H]⁻ 377.1171; found m/z 377.1169.

### Example 17. Synthesis of tert-Butyl 5-((4-(ethoxycarbonyl)phenyl)carbamothioyl)-4-hydroxy-6-oxo-3,6-dihydropyridine-1(2H)-carboxylate, 5v

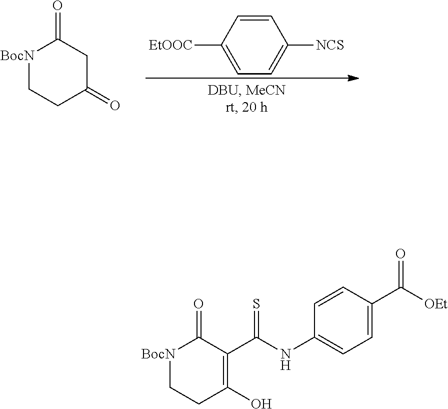

Synthesised according to GP1 using tert-butyl 2,4-dioxopiperidine-1-carboxylate (300 mg, 1.41 mmol) and ethyl 4-isothiocyanatobenzoate (292 mg, 1.41 mmol). Purified by silica gel chromatography using a 40 g SiO₂ cartridge and a linear gradient of 0-45% EtOAc in heptane over 14 CV to give 5v (319 mg, 54%) as a white solid.

¹H NMR (400 MHz, Chloroform-d) δ 13.81 (s, 1H), 8.09 (d, 2H), 7.59 (d, 2H), 4.38 (q, J=7.1 Hz, 2H), 3.85 (t, J=6.6 Hz, 2H), 2.83 (t, J=6.6 Hz, 2H), 1.56 (s, 9H), 1.39 (t, J=7.1 Hz, 3H);

¹³C NMR (101 MHz, Chloroform-d) δ 189.8, 186.8, 168.1, 166.3, 152.2, 142.0, 130.8, 129.4, 125.7, 102.6, 84.5, 61.5, 40.8, 32.2, 28.5, 14.8; HRMS (ESI) calculated for C₂₀H₂₅N₂O₆S⁺ [M+H]⁺ 421.1433; found m/z 421.1429.

### Example 18. Synthesis of tert-Butyl 4-hydroxy-3-methyl-6-oxo-5-(phenylcarbamothioyl)-3,6-dihydropyridine-1(2H)-carboxylate, 5y

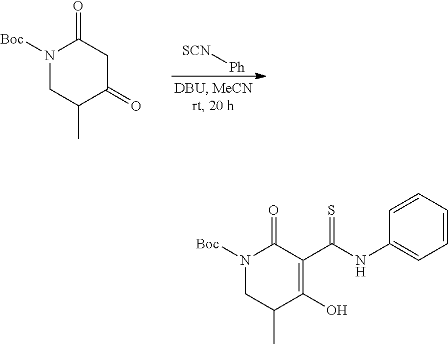

Synthesised according to GP1 using tert-butyl 5-methyl-2,4-dioxopiperidine-1-carboxylate (500 mg, 2.20 mmol) and isothiocyanatobenzene (263 PL, 2.20 mmol). Purified by silica gel chromatography using a 40 g SiO₂ cartridge and a linear gradient of 0-40% EtOAc in heptane over 15 CV to give 5y (585 mg, 73%) as an off-white solid.

¹H NMR (400 MHz, CDCl₃) δ 13.61 (s, 1H), 7.42 (d, J=4.3 Hz, 4H), 7.35-7.27 (m, 1H), 3.86 (dd, J=13.0, 4.6 Hz, 1H), 3.63 (dd, J=13.0, 7.1 Hz, 1H), 2.86 (td, J=7.1, 4.8 Hz, 1H), 1.56 (s, 9H), 1.34 (d, J=7.0 Hz, 3H);

¹³C NMR (101 MHz, CDCl₃) δ 189.7, 189.3, 167.5, 152.1, 137.6, 129.0, 127.4, 125.9, 100.9, 83.8, 46.6, 35.9, 28.1, 14.4;

HRMS (ESI) calculated for C₁₈H₂₃N₂O₄S⁺ [M+H]⁺ 363.1378; found m/z 363.1379.

### Example 19. Synthesis of Sodium hydroxy(pyridin-4-yl)methanesulfonate, 11

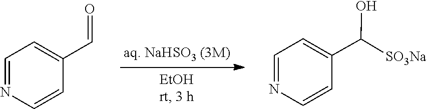

To a solution of 4-formylpyridine (600 uL, 6.37 mmol, 1.00 eq) in EtOH (12.7 mL) was added aq. 3 m NaHSO₃ (2.14 mL, 6.43 mmol, 1.01 eq) and the mixture stirred at room temperature for 3 hours. Toluene (10 mL) was added to the reaction mixture for azeotropic removal of water, and the solvent was evaporated to dryness. Additional toluene (10 mL) was added and the solvent was evaporated again to dryness to afford sodium hydroxy(pyridin-4-yl)methanesulfonate 11 (1.10 g, 82%) as a white solid which was used without further purification.

¹H NMR (400 MHz, DMSO-d₆) δ 8.55-8.34 (m, 2H), 7.52-7.25 (m, 2H), 6.25 (s, 1H), 5.01 (s, 1H).

### Example 20. Synthesis of tert-Butyl 4-oxo-3-(phenylamino)-2-(pyridin-4-yl)-1,4,6,7-tetrahydro-5H-pyrrolo[3,2-c]pyridine-5-carboxylate, 7a

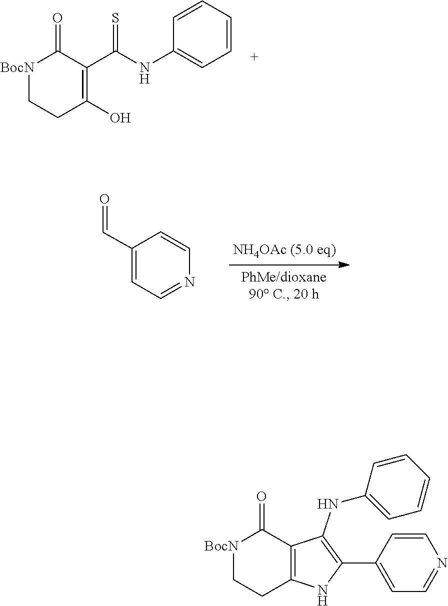 [SMILES: CC(C)(C)OC(=O)N1CCC(O)=C(C(=S)Nc2ccccc2)C1=O ; O=Cc1ccncc1 ; CC(C)(C)OC(=O)N1CCc2[nH]c(-c3ccncc3)c(Nc3ccccc3)c2C1=O]

Small-scale: Synthesised according to GP2 using 5a (100 mg, 0.287 mmol) and isonicotinaldehyde (40.6 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7a (81 mg, 70%) as an off-white solid.

1 g scale: The reaction was carried out according to GP2 using 5a (1.00 g, 2.87 mmol) and isonicotinaldehyde (406 μL, 4.31 mmol) to give 7a (739 mg, 64%) as an off-white solid.

¹H NMR (400 MHz, Chloroform-d) 11.25 (s, 1H), 8.34-8.24 (m, 2H), 7.39-7.28 (m, 2H), 7.23 (s, 1H), 7.12-6.99 (m, 2H), 6.75 (tt, J=7.4, 1.1 Hz, 1H), 6.70-6.58 (m, 2H), 4.04 (t, J=6.3 Hz, 2H), 2.85 (t, J=6.3 Hz, 2H), 1.52 (s, 9H).

¹³C NMR (101 MHz, Chloroform-d) δ 164.0, 152.8, 148.9, 143.3, 139.3, 138.8, 129.2, 128.8, 120.1, 119.0, 118.6, 116.1, 108.8, 83.0, 45.3, 28.2, 22.8.

HRMS (ESI) calculated for C₂₃H₂₅N₄O₃⁺ [M+H]⁺ 405.1927; found m/z 405.1929.

### Example 21. Synthesis of tert-Butyl-4-oxo-3-(phenylamino)-2-(pyridin-2-yl)-1,4,6,7-tetrahydro-5H-pyrrolo[3,2-c]pyridine-5-carboxylate, 7b

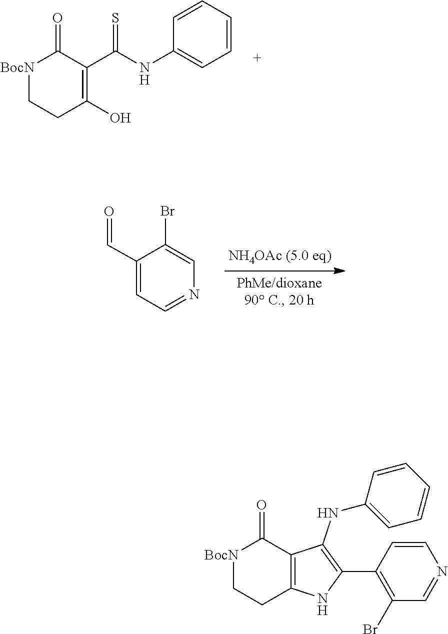 [SMILES: CC(C)(C)OC(=O)N1CCC(O)=C(C(=S)Nc2ccccc2)C1=O ; O=Cc1ccncc1Br ; CC(C)(C)OC(=O)N1CCc2[nH]c(-c3ccncc3Br)c(Nc3ccccc3)c2C1=O]

Synthesised according to GP2 using tert-butyl 5a (100 mg, 0.287 mmol) and 3-bromoisonicotinaldehyde (80.1 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7b (75 mg, 54%) as a yellow solid.

¹H NMR (400 MHz, Chloroform-d) δ 9.29 (s, 1H), 8.60 (s, 1H), 8.08 (d, J=5.1 Hz, 1H), 7.18 (d, J=5.1 Hz, 1H), 6.98 (t, J=7.7 Hz, 2H), 6.69 (t, J=7.3 Hz, 1H), 6.61 (d, J=8.0 Hz, 2H), 4.12 (t, J=6.2 Hz, 2H), 2.96 (t, J=6.3 Hz, 2H), 1.55 (s, 9H);

¹³C NMR (101 MHz, Chloroform-d) δ 164.0, 152.9, 152.2, 147.5, 142.0, 139.4, 137.3, 130.4, 128.6, 124.1, 120.5, 116.7, 112.9, 107.1, 83.0, 45.1, 28.2, 23.1;

HRMS (ESI) calculated for C₂₃H₂₄BrN₄O₃⁺ [M+H]⁺ 485.1014; found m/z 485.1012.

### Example 22. Synthesis of tert-Butyl 2-(3-methylpyridin-4-yl)-4-oxo-3-(phenylamino)-1,4,6,7-tetrahydro-5H-pyrrolo[3,2-c]pyridine-5-carboxylate, 7c

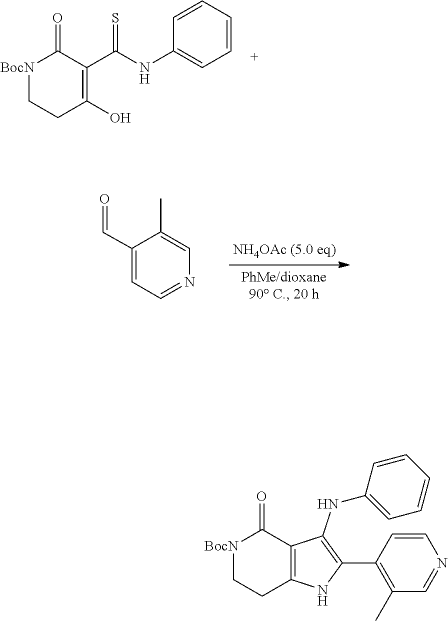

Synthesised according to GP2 using 5a (100 mg, 0.287 mmol) and 3-methylisonicotinaldehyde (52.2 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7c (50 mg, 42%) as a yellow solid.

¹H NMR (400 MHz, Chloroform-d) δ 9.31 (s, 1H), 8.11 (s, 2H), 7.40 (s, 1H), 7.10 (d, J=5.2 Hz, 1H), 6.83 (t, J=7.7 Hz, 2H), 6.56 (t, J=7.3 Hz, 1H), 6.45 (d, J=8.0 Hz, 2H), 4.04 (t, J=6.3 Hz, 2H), 2.86 (t, J=6.3 Hz, 2H), 2.10 (s, 3H), 1.49 (s, 9H);

¹³C NMR (101 MHz, Chloroform-d) δ 164.2, 153.1, 150.5, 145.9, 142.9, 141.2, 137.3, 131.5, 128.4, 128.4, 122.0, 119.8, 116.0, 115.1, 107.6, 82.69, 45.2, 28.2, 23.0, 17.4.

HRMS (ESI) calculated for C₂₄H₂₇rN₄O₃⁺ [M+H]⁺ 419.2083; found m/z 419.2083.

### Example 23. Synthesis of tert-Butyl-2-(2-bromopyridin-4-yl)-4-oxo-3-(phenylamino)-1,4,6,7-tetrahydro-5H-pyrrolo[3,2-c]pyridine-5-carboxylate, 7d

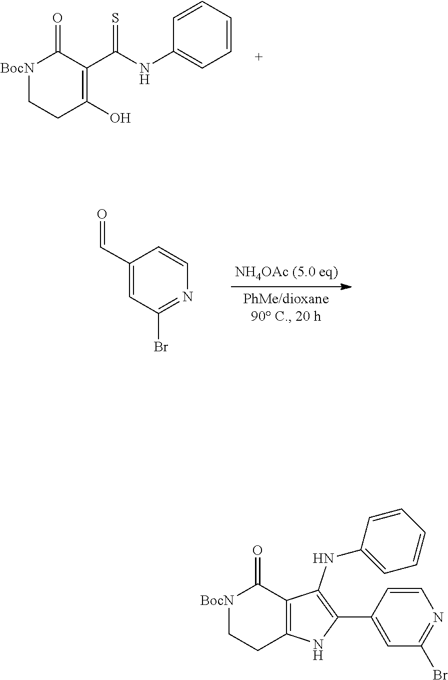 [SMILES: CC(C)(C)OC(=O)N1CCC(O)=C(C(=S)Nc2ccccc2)C1=O ; O=Cc1ccnc(Br)c1 ; CC(C)(C)OC(=O)N1CCc2[nH]c(-c3ccnc(Br)c3)c(Nc3ccccc3)c2C1=O]

Synthesised according to GP2 using 5a (100 mg, 0.287 mmol) and 2-bromoisonicotinaldehyde (80.1 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7d (56 mg, 40%) as a yellow solid.

¹H NMR (400 MHz, Chloroform-d) δ 9.52 (s, 1H), 8.01 (d, J=5.4 Hz, 1H), 7.36 (s, 1H), 7.15-7.03 (m, 3H), 6.79 (t, J=7.2 Hz, 1H), 6.62 (d, J=7.9 Hz, 2H), 4.11 (t, J=6.4 Hz, 2H), 2.95 (d, J=6.4 Hz, 2H), 1.53 (s, 9H);

¹³C NMR (101 MHz, Chloroform-d) δ 164.0, 152.7, 150.3, 149.5, 142.5, 142.1, 140.9, 139.0, 130.2, 129.0, 121.8, 120.7, 118.1, 116.6, 116.4, 108.9, 83.4, 45.2, 28.1, 22.9;

HRMS (ESI) calculated for C₂₃H₂₄BrN₄O₃⁺ [M+H]⁺ 483.1032; found m/z 483.1028.

### Example 24. Synthesis of tert-Butyl-2-(2-methoxypyridin-4-yl)-4-oxo-3-(phenylamino)-1,4,6,7-tetrahydro-5H-pyrrolo[3,2-c]pyridine-5-carboxylate, 7e

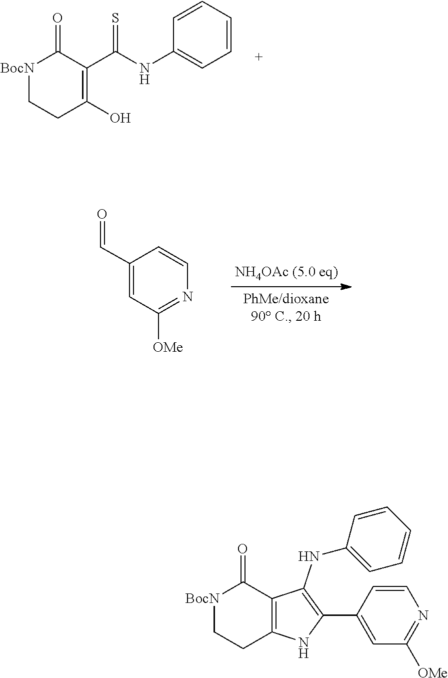 [SMILES: CC(C)(C)OC(=O)N1CCC(O)=C(C(=S)Nc2ccccc2)C1=O ; COc1cc(C=O)ccn1 ; COc1cc(-c2[nH]c3c(c2Nc2ccccc2)C(=O)N(C(=O)OC(C)(C)C)CC3)ccn1]

Synthesised according to GP2 using 5a (100 mg, 0.287 mmol) and 2-methoxyisonicotinaldehyde (40.9 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7e (100 mg, 80%) as an off-white solid.

¹H NMR (400 MHz, Chloroform-d) δ 10.00 (s, 1H), 7.90 (d, J=5.6 Hz, 1H), 7.10-7.01 (m, 3H), 6.95 (d, J=5.6 Hz, 1H), 6.77-6.69 (m, 2H), 6.62 (d, J=8.0 Hz, 2H), 4.03 (t, J=6.3 Hz, 2H), 2.85 (t, J=6.3 Hz, 2H), 1.51 (s, 9H);

¹³C NMR (101 MHz, Chloroform-d) δ 164.6, 164.1, 152.8, 146.7, 143.5, 141.3, 138.2, 128.8, 128.4, 119.9, 119.0, 115.9, 112.87, 108.9, 104.2, 83.0, 53.6, 45.3, 28.1, 22.8;

HRMS (ESI) calculated for C₂₄H₂₇N₄O₄⁺ [M+H]⁺ 435.2032; found m/z 435.2035.

### Example 25. Synthesis of tert-Butyl-2-(2-fluoropyridin-4-yl)-4-oxo-3-(phenylamino)-1,4,6,7-tetrahydro-5H-pyrrolo[3,2-c]pyridine-5-carboxylate, 7f

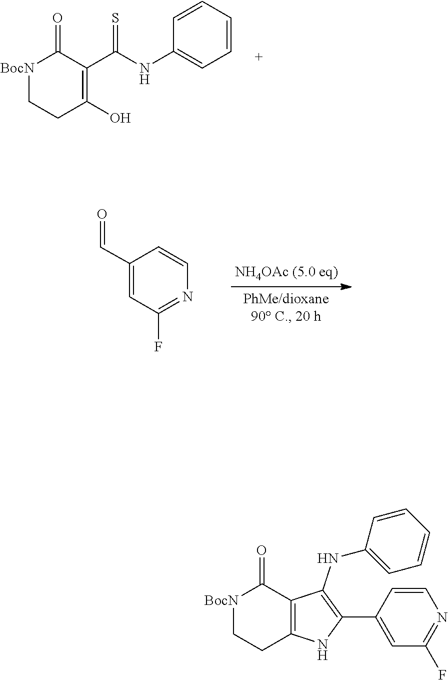 [SMILES: CC(C)(C)OC(=O)N1CCC(O)=C(C(=S)Nc2ccccc2)C1=O ; O=Cc1ccnc(F)c1 ; CC(C)(C)OC(=O)N1CCc2[nH]c(-c3ccnc(F)c3)c(Nc3ccccc3)c2C1=O]

Synthesised according to GP2 using 5a (100 mg, 0.287 mmol) and 2-fluoroisonicotinaldehyde (53.9 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7f (72 mg, 59%) as a yellow solid.

¹H NMR (400 MHz, Chloroform-d) δ 10.02 (s, 1H), 7.89 (d, J=5.5 Hz, 1H), 7.21-7.11 (m, 2H), 7.07 (t, J=7.7 Hz, 2H), 6.88 (d, J=1.4 Hz, 1H), 6.77 (t, J=7.3 Hz, 1H), 6.63 (d, J=7.9 Hz, 2H), 4.06 (t, J=6.3 Hz, 2H), 2.90 (t, J=6.3 Hz, 2H), 1.52 (s, 9H);

¹³C NMR (101 MHz, Chloroform-d) δ 164.2, 152.8, 147.1 (d, J=15.1 Hz), 143.8 (d, J=9.2 Hz), 143.0, 139.15, 130.0, 129.1, 120.6, 117.9, 116.9, 116.3, 109.0, 103.5 (d, J=39.2 Hz), 83.4, 45.4, 28.2, 22.8;

¹⁹F NMR (376 MHz, Chloroform-d) δ −69.1;

HRMS (ESI) calculated for C₂₃H₂₄FN₄O₃⁺ [M+H]⁺ 423.1833; found m/z 428.1830.

### Example 26. Synthesis of tert-Butyl 4-oxo-3-(phenylamino)-2-(pyridin-2-yl)-1,4,6,7-tetrahydro-5H-pyrrolo[3,2-c]pyridine-5-carboxylate, 7g

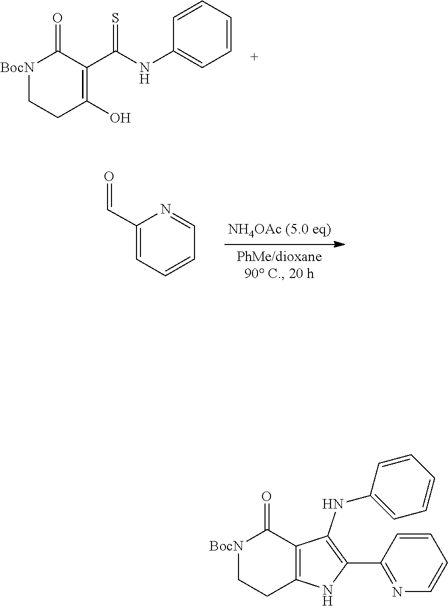 [SMILES: CC(C)(C)OC(=O)N1CCC(O)=C(C(=S)Nc2ccccc2)C1=O ; O=Cc1ccccn1 ; CC(C)(C)OC(=O)N1CCc2[nH]c(-c3ccccn3)c(Nc3ccccc3)c2C1=O]

Synthesised according to GP2 using 5a (100 mg, 0.287 mmol) and picolinaldehyde (40.9 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7g (57 mg, 49%) as an off-white solid.

¹H NMR (400 MHz, Chloroform-d) δ 10.01 (br s, 1H), 8.43 (d, J=5.0 Hz, 1H), 7.46 (td, J=7.7, 1.8 Hz, 1H), 7.23 (s, 1H), 7.18 (d, J=8.2 Hz, 1H), 7.11 (t, J=8.5, 7.2 Hz, 2H), 6.99 (ddd, J=7.6, 4.9, 1.1 Hz, 1H), 6.78 (t, J=7.3 Hz, 1H), 6.73 (d, J=7.6 Hz, 2H), 4.10 (t, J=6.3 Hz, 2H), 2.91 (t, J=6.3 Hz, 2H), 1.55 (s, 9H);

¹³C NMR (101 MHz, Chloroform-d₃) δ 163.8, 153.0, 148.6, 148.0, 143.9, 136.1, 136.7, 1289, 127.3, 121.8, 121.0, 120.4, 119.8, 116.0, 109.3, 82.7, 45.2, 28.2, 22.9;

HRMS (ESI) calculated for C₂₃H₂₅N₄O₃⁺ [M+H]⁺ 405.1927; found m/z 405.1928.

### Example 27. Synthesis of tert-Butyl-2-(5-bromopyridin-2-yl)-4-oxo-3-(phenylamino)-1,4,6,7-tetrahydro-5H-pyrrolo[3,2-c]pyridine-5-carboxylate, 7h

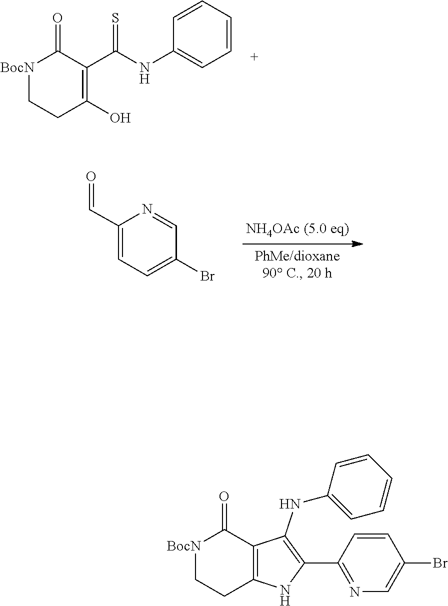 [SMILES: CC(C)(C)OC(=O)N1CCC(O)=C(C(=S)Nc2ccccc2)C1=O ; O=Cc1ccc(Br)cn1 ; CC(C)(C)OC(=O)N1CCc2[nH]c(-c3ccc(Br)cn3)c(Nc3ccccc3)c2C1=O]

Synthesised according to GP2 using 5a (100 mg, 0.287 mmol) and 5-bromopicolinaldehyde (40.9 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7h (72 mg, 52%) as a yellow solid.

¹H NMR (400 MHz, Chloroform-d) δ 9.59 (s, 1H), 8.48 (d, J=2.3 Hz, 1H), 7.54 (dd, J=8.6, 2.4 Hz, 1H), 7.18-7.08 (m, 2H), 7.00 (d, J=8.6 Hz, 1H), 6.85-6.76 (m, 1H), 6.75-6.68 (m, 2H), 4.11 (t, J=6.3 Hz, 2H), 2.94 (t, J=6.3 Hz, 2H), 1.56 (s, 9H);

¹³C NMR (101 MHz, Chloroform-d) δ 163.7, 153.0, 149.0, 146.8, 143.5, 139.0, 137.0, 129.0, 127.9, 121.9, 120.9, 120.1, 116.4, 116.0, 109.4, 82.9, 45.1, 28.2, 22.9;

HRMS (ESI) calculated for C₂₃H₂₄BrN₄O₃⁺ [M+H]⁺ 483.1032; found m/z 483.1026.

### Example 28. Synthesis of tert-Butyl 2-(5-methylpyridin-2-yl)-4-oxo-3-(phenylamino)-1,4,6,7-tetrahydro-5H-pyrrolo[3,2-c]pyridine-5-carboxylate, 7i

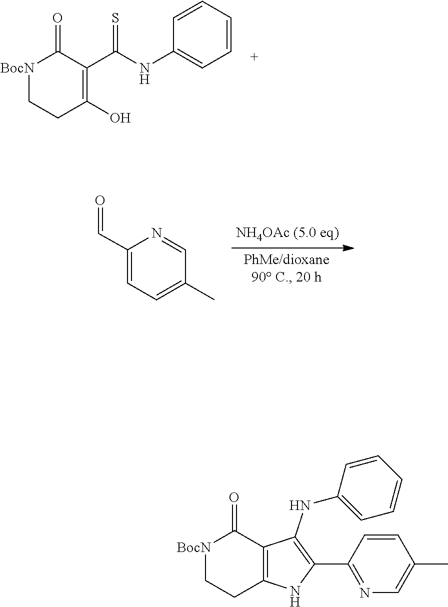

Synthesised according to GP2 using 5a (100 mg, 0.287 mmol) and 5-methylpicolinaldehyde (52.2 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7i (76 mg, 63%) as an off-white solid.

¹H NMR (400 MHz, Chloroform-d) δ 10.60 (s, 1H), 8.36 (d, J=2.2 Hz, 1H), 7.42-7.35 (m, 1H), 7.30-7.15 (m, 3H), 6.85 (dd, J=20.3, 7.6 Hz, 3H), 4.17 (t, J=6.3 Hz, 2H), 2.92 (t, J=6.3 Hz, 2H), 2.35 (s, 3H), 1.64 (s, 9H);

¹³C NMR (101 MHz, Chloroform-d) δ 163.9, 153.1, 148.1, 146.3, 144.2, 137.4, 136.6, 130.0, 128.8, 126.6, 122.2, 120.6, 119.62, 115.9, 109.3, 82.7, 45.2, 28.2, 22.8, 18.2;

HRMS (ESI) calculated for C₂₄H₂₇N₄O₃⁺ [M+H]⁺ 419.2083; found m/z 419.2086.

### Example 29. Synthesis of tert-Butyl 4-oxo-3-(phenylamino)-2-(quinolin-2-yl)-1,4,6,7-tetrahydro-5H-pyrrolo[3,2-c]pyridine-5-carboxylate, 7j

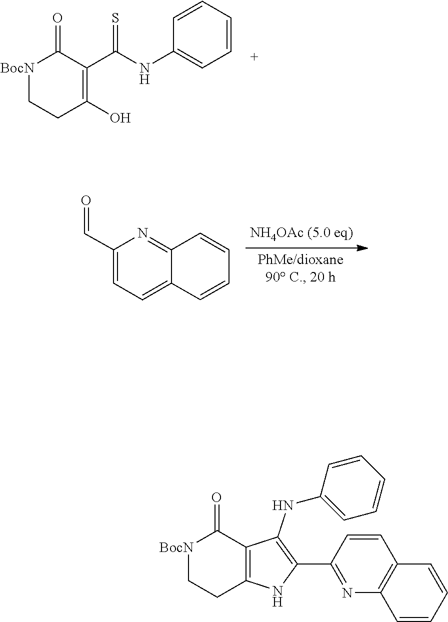 [SMILES: CC(C)(C)OC(=O)N1CCC(O)=C(C(=S)Nc2ccccc2)C1=O ; O=Cc1ccc2ccccc2n1 ; CC(C)(C)OC(=O)N1CCc2[nH]c(-c3ccc4ccccc4n3)c(Nc3ccccc3)c2C1=O]

Synthesised according to GP2 using 5a (100 mg, 0.287 mmol) and quinoline-2-carbaldehyde (67.6 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7j (70 mg, 53%) as an orange solid.

¹H NMR (400 MHz, Chloroform-d₃) δ 7.96 (d, J=8.5 Hz, 1H), 7.90 (d, J=8.8 Hz, 1H), 7.73-7.61 (m, 2H), 7.48 (d, J=8.7 Hz, 1H), 7.43 (t, J=7.5 Hz, 1H), 7.34 (s, 1H), 7.13-7.05 (m, 2H), 6.85-6.74 (m, 3H), 4.09 (t, J=6.3 Hz, 2H), 2.84 (t, J=6.3 Hz, 2H), 1.55 (s, 9H);

¹³C NMR (101 Mz, Chloroform-d) δ 163.2, 152.4, 143.9, 137.5, 135.8, 129.2, 128.3, 127.4, 127.1, 126.0, 124.9, 119.4, 119.0, 115.4, 108.8, 82.1, 44.5, 27.6, 22.2;

HRMS (ESI) calculated for C₂₇H₂₇N₄O₃⁺ [M+H]⁺ 455.2083; found m/z 455.2086.

### Example 30. Synthesis of tert-Butyl 4-oxo-3-(phenylamino)-2-(pyridin-3-yl)-1,4,6,7-tetrahydro-5H-pyrrolo[3,2-c]pyridine-5-carboxylate, 7k

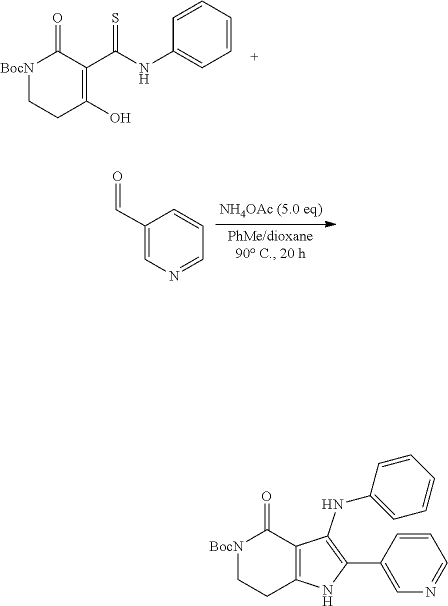 [SMILES: CC(C)(C)OC(=O)N1CCC(O)=C(C(=S)Nc2ccccc2)C1=O ; O=Cc1cccnc1 ; CC(C)(C)OC(=O)N1CCc2[nH]c(-c3cccnc3)c(Nc3ccccc3)c2C1=O]

Synthesised according to GP2 using 5a (100 mg, 0.287 mmol) and 3-formylpyridine (40.4 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7k (22 mg, 19% yield) as a yellow solid.

¹H NMR (400 MHz, Chloroform-d) δ 11.23 (s, 1H), 9.00 (s, 1H), 8.20 (d, J=4.9 Hz, 1H), 7.79 (dd, J=8.1, 2.0 Hz, 1H), 7.19-6.97 (m, 4H), 6.71 (t, J=7.3 Hz, 1H), 6.62 (d, J=7.9 Hz, 2H), 4.03 (t, J=6.3 Hz, 2H), 2.88 (t, J=6.3 Hz, 2H), 1.53 (s, 9H).

¹³C NMR (101 MHz, Chloroform-d) δ 164.0, 152.92, 143.7, 143.4, 138.4, 134.1, 129.2, 128.9, 127.5, 124.1, 119.8, 117.5, 115.9, 108.6, 82.8, 45.4, 28.2, 22.7.

HRMS (ESI) calculated for C₂₃H₂₅N₄O₃⁺ [M+H]⁺ 405.1927; found m/z 405.1927.

### Example 31. Synthesis of tert-Butyl 4-oxo-3-(phenylamino)-2-(thiazol-2-yl)-1,4,6,7-tetrahydro-5H-pyrrolo[3,2-c]pyridine-5-carboxylate, 7l

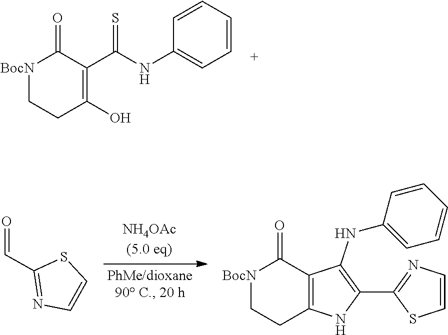 [SMILES: CC(C)(C)OC(=O)N1CCC(O)=C(C(=S)Nc2ccccc2)C1=O ; O=Cc1nccs1 ; CC(C)(C)OC(=O)N1CCc2[nH]c(-c3nccs3)c(Nc3ccccc3)c2C1=O]

Synthesised according to GP2 using 5a (100 mg, 0.287 mmol) and thiazole-2-carbaldehyde (37.8 μL, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7l (40 mg, 34%) as a yellow solid.

¹H NMR (400 MHz, Chloroform-d) δ 10.57 (s, 1H), 7.64 (d, J=3.3 Hz, 1H), 7.21-7.12 (m, 3H), 7.10 (s, 1H), 6.86 (t, J=7.3 Hz, 1H), 6.79 (d, J=8.0 Hz, 2H), 4.10 (t, J=6.3 Hz, 2H), 2.91 (t, J=6.3 Hz, 2H), 1.55 (s, 9H);

¹³C NMR (101 MHz, Chloroform-d₃) δ 163.2, 157.0, 153.0, 143.0, 139.1, 138.7, 129.0, 120.8, 118.4, 118.2, 116.9, 109.2, 83.0, 45.0, 28.2, 22.9;

HRMS (ESI) calculated for C₂₁H₂₂N₄O₃SNa⁺ [M+Na]⁺ 433.1310; found m/z 433.1308.

### Example 32. Synthesis of tert-Butyl 2-(1-methyl-1H-imidazol-2-yl)-4-oxo-3-(phenylamino)-1,4,6,7-tetrahydro-5H-pyrrolo[3,2-c]pyridine-5-carboxylate, 7m

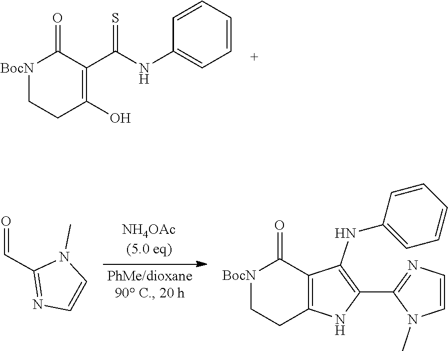

Synthesised according to GP2 using 5a (100 mg, 0.287 mmol) and 1-methyl-1H-imidazole-2-carbaldehyde (47.4 mg, 0.431 mmol). Purified by silica gel chromatography using a 24 g SiO₂ cartridge and a linear gradient of 20-100% EtOAc in heptane over 10 CV, followed by a linear gradient of 0-10% MeOH in DCM over 20 CV to give 7m (40 mg, 34%) as a yellow solid.

¹H NMR (400 MHz, Chloroform-d) δ 12.75 (br s, 1H), 7.04-6.94 (m, 3H), 6.73 (t, J=7.3 Hz, 1H), 6.69-6.63 (m, 3H), 4.10 (t, J=6.3 Hz, 2H), 3.34 (s, 3H), 2.94 (t, J=6.3 Hz, 2H), 1.56 (s, 9H);

¹³C NMR (101 Mz, Chloroform-d) δ 164.5, 153.1, 143.3, 141.8, 138.1, 128.8, 128.6, 126.4, 121.0, 120.0, 115.2, 106.8, 106.5, 82.7, 45.4, 33.6, 28.2, 22.6;

HRMS (ESI) calculated for C₂₂H₂₆N₅O₃⁺ [M+H]⁺ 408.2036; found m/z 408.2035.

### Example 33. Synthesis of tert-Butyl 4-oxo-3-(phenylamino)-2-(4-(trifluoromethyl)phenyl)-1,4,6,7-tetrahydro-5H-pyrrolo[3,2-c]pyridine-5-carboxylate, 7n

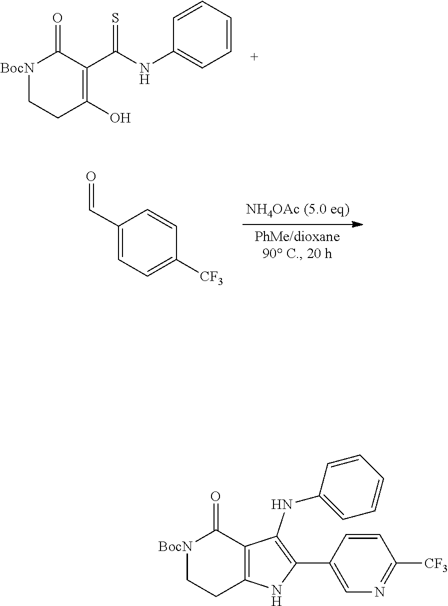 [SMILES: CC(C)(C)OC(=O)N1CCC(O)=C(C(=S)Nc2ccccc2)C1=O ; O=Cc1ccc(C(F)(F)F)cc1 ; CC(C)(C)OC(=O)N1CCc2[nH]c(-c3ccc(C(F)(F)F)nc3)c(Nc3ccccc3)c2C1=O]

Synthesised according to GP2 using 5a (100 mg, 0.287 mmol) and 4-(trifluoromethyl)benzaldehyde (60.0 μL, 0.431 mmol). Purified by silica gel chromatography using a 24 g SiO₂ cartridge and a linear gradient of 0-55% EtOAc in heptane over 13 CV to give 7n (93 mg, 69%) as a yellow solid.

¹H NMR (400 MHz, DMSO-d₆) δ 11.96 (s, 1H), 7.77 (d, J=8.1 Hz, 2H), 7.64 (d, J=8.1 Hz, 2H), 7.35 (s, 1H), 7.02 (t, J=7.6 Hz, 1H), 6.63-6.55 (m, 3H), 3.96 (t, J=6.0 Hz, 2H), 2.95 (t, J=6.0 Hz, 2H), 1.44 (s, 9H);

¹³C NMR (101 MHz, DMSO-d₆) δ 161.7, 153.1, 146.1, 139.0, 135.3, 128.7, 125.4, 124.7, 124.6, 122.6, 117.6, 114.0, 109.4, 81.3, 45.1, 27.8, 22.3;

¹⁹F NMR (376 MHz, DMSO-d₆) δ −60.7;

HRMS (ESI) calculated for C₂₅H₂₅F₃N₃O₃⁺ [M+H]⁺ 472.1848; found m/z 472.1845.

### Example 34. Synthesis of tert-Butyl 2-(4-nitrophenyl)-4-oxo-3-(phenylamino)-1,4,6,7-tetrahydro-5H-pyrrolo[3,2-c]pyridine-5-carboxylate, 7o

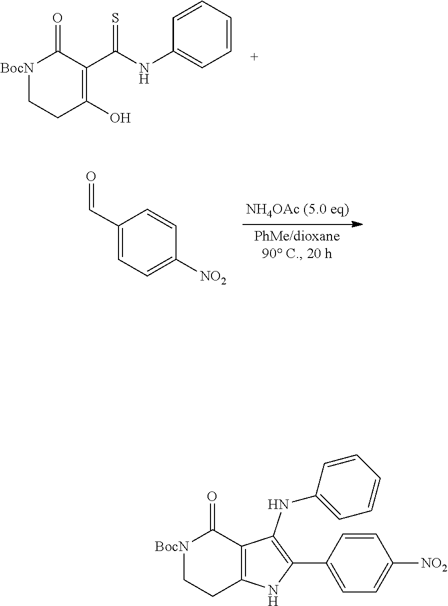 [SMILES: CC(C)(C)OC(=O)N1CCC(O)=C(C(=S)Nc2ccccc2)C1=O ; O=Cc1ccc([N+](=O)[O-])cc1 ; CC(C)(C)OC(=O)N1CCc2[nH]c(-c3ccc([N+](=O)[O-])cc3)c(Nc3ccccc3)c2C1=O]

Synthesised according to GP2 using 5a (100 mg, 0.287 mmol) and 4-nitrobenzaldehyde (65.1 mg, 0.431 mmol). Purified by silica gel chromatography using a 24 g SiO₂ cartridge and a linear gradient of 0-90% EtOAc in heptane over 10 CV to give 7o (95 mg, 74%) as a red solid.

¹H NMR (400 MHz, DMSO-d₆) δ 12.10 (s, 1H), 8.14 (d, J=8.6 Hz, 2H), 7.77 (d, J=8.6 Hz, 2H), 7.51 (s, 1H), 7.04 (t, J=7.6 Hz, 2H), 6.71-6.54 (m, 3H), 3.97 (t, J=6.2 Hz, 2H), 2.98 (t, J=6.3 Hz, 2H), 1.45 (s, 9H);

¹³C NMR (101 MHz, DMSO-d₆) δ 162.2, 153.5, 145.8, 144.6, 140.7, 138.2, 129.2, 127.2, 124.9, 124, 122.2, 118.6, 114.8, 109.84, 81.9, 45.5, 28.3, 22.8;

HRMS (ESI) calculated for C₂₄H₂₅N₄O₅⁺ [M+H]⁺ 449.1823; found m/z 449.1823.

### Example 35. Synthesis of tert-Butyl 2-(4-cyanophenyl)-4-oxo-3-(phenylamino)-1,4,6,7-tetrahydro-5H-pyrrolo[3,2-c]pyridine-5-carboxylate, 7p

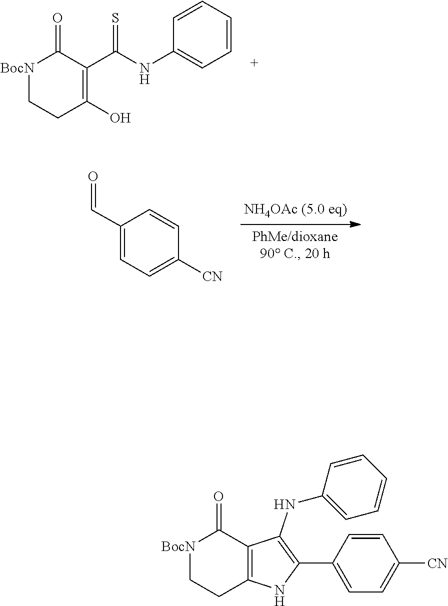 [SMILES: CC(C)(C)OC(=O)N1CCC(O)=C(C(=S)Nc2ccccc2)C1=O ; N#Cc1ccc(C=O)cc1 ; CC(C)(C)OC(=O)N1CCc2[nH]c(-c3ccc(C#N)cc3)c(Nc3ccccc3)c2C1=O]

Synthesised according to GP2 using 5a (100 mg, 0.287 mmol) and 4-formylbenzonitrile (56.5 mg, 0.431 mmol). Purified by silica gel chromatography using a 24 g SiO₂ cartridge and a linear gradient of 10-100% EtOAc in heptane over 20 CV to give 7p (76 mg, 62%) as a yellow solid.

¹H NMR (400 MHz, Chloroform-d) δ 9.83 (s, 1H), 7.49 (d, J=8.7 Hz, 2H), 7.40 (d, J=8.6 Hz, 2H), 7.06-6.95 (m, 3H), 6.78-6.67 (m, 1H), 6.67-6.50 (m, 2H), 4.03 (t, J=6.3 Hz, 2H), 2.89 (t, J=6.3 Hz, 2H), 1.50 (s, 9H);

¹³C NMR (101 MHz, Chloroform-d) δ 164.3, 152.6, 143.4, 138.3, 135.8, 132.2, 128.8, 127.6, 124.8, 120.0, 119.8, 119.3, 115.8, 108.8, 108.0, 83.1, 45.4, 28.1, 22.8;

HRMS (ESI) calculated for C₂₅H₂₃N₄O₃⁻ [M−H]⁻ 427.1770; found m/z 427.1773.

### Example 36. Synthesis of tert-Butyl 2-(3,5-difluorophenyl)-4-oxo-3-(phenylamino)-1,4,6,7-tetrahydro-5H-pyrrolo[3,2-c]pyridine-5-carboxylate, 7q

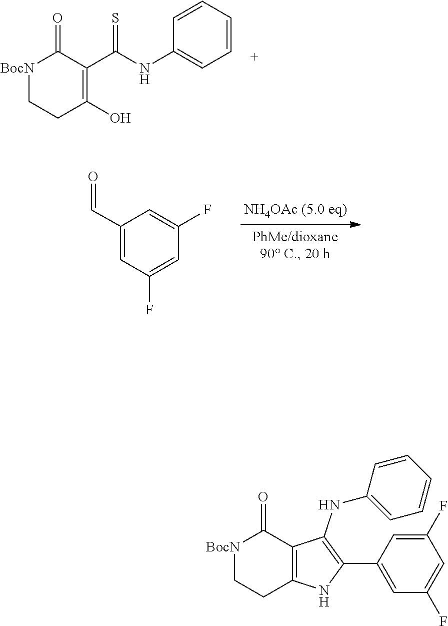 [SMILES: CC(C)(C)OC(=O)N1CCC(O)=C(C(=S)Nc2ccccc2)C1=O ; O=Cc1cc(F)cc(F)c1 ; CC(C)(C)OC(=O)N1CCc2[nH]c(-c3cc(F)cc(F)c3)c(Nc3ccccc3)c2C1=O]

Synthesised according to GP2 using 5a (100 mg, 0.287 mmol) and 3,5-difluorobenzaldehyde (40.0 μL, 0.431 mmol). Purified by silica gel chromatography using a 24 g SiO₂ cartridge and a linear gradient of 0-55% EtOAc in heptane over 10 CV to give 7q (82 mg, 65%) as a yellow solid.

¹H NMR (400 MHz, DMSO-d₆) δ 11.90 (br s, 1H), 7.34 (s, 1H), 7.30-7.23 (m, 2H), 7.10-7.00 (m, 2H), 6.97 (t, J=9.0 Hz, 1H), 6.65-6.56 (m, 3H), 3.95 (t, J=6.2 Hz, 2H), 2.94 (t, J=6.2 Hz, 2H), 1.44 (s, 9H);

¹³C NMR (101 MHz, DMSO-d₆) δ 164.3, 162.0, 146.4, 139.4, 134.8, 129.2, 125.2, 122.5, 118.2, 114.4, 110.0, 107.7, 107.4, 81.8, 45.5, 28.3, 22.7;

¹⁹F NMR (376 MHz, DMSO-d₆) δ −110.0;

HRMS (ESI) calculated for C₂₄H₂₄F₂N₃O₃⁺ [M+H]⁺ 440.1786; found m/z 440.1786.

### Example 37. Synthesis of tert-Butyl 4-oxo-3-(phenylamino)-2-(pyridin-3-yl)-1,4,6,7-tetrahydro-5H-pyrrolo[3,2-c]pyridine-5-carboxylate, 7a

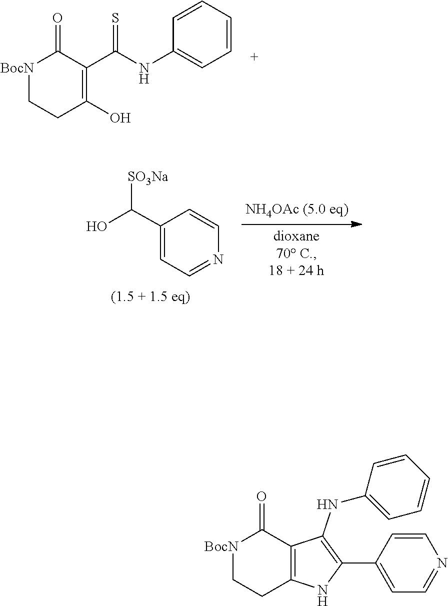

Due to solubility issues with bisulfite adduct 11, the reaction was carried out in a one-pot manner with 1,4-dioxane as the solvent, rather than following GP2.

A mixture of 5a (100 mg, 0.287 mmol), 11 (90.9 mg, 0.431 mmol), and NH₄OAc (111 mg, 1.44 mmol) in 1,4-dioxane (2.00 mL) was heated to 70° C. for 18 hours. Additional 11 (90.9 mg, 0.431 mmol) was added and the mixture stirred at 70° C. for a further 24 hours. The reaction mixture was quenched with saturated aqueous NaHCO₃ (10 mL) and extracted with EtOAc (3×10 mL). The combined organic extracts were washed with brine, dried over Na₂SO₄, filtered, and concentrated under reduced pressure. Purification was carried out by silica gel chromatography, eluting with n-heptane/EtOAc (80:20 to 0:100) and then DCM/MeOH (100:0 to 90:10) to afford 7a (35 mg, 30% yield) as a white solid.

¹H NMR (400 MHz, Chloroform-d) δ 10.20 (s, 1H), 8.37-8.27 (m, 2H), 7.32-7.22 (m, 3H), 7.11-7.00 (m, 2H), 6.76 (tt, J=7.3, 1.1 Hz, 1H), 6.70-6.61 (m, 2H), 4.08 (t, J=6.3 Hz, 2H), 2.91 (t, J=6.3 Hz, 2H), 1.54 (s, 9H);

LCMS (ESI) calculated for C₂₃H₂₅N₄O₃⁺ [M+H]⁺ 405.2; found m/z 405.3.

The NMR/MS spectra matched that obtained previously.

### Example 38. Synthesis of tert-Butyl 3-((2-chlorophenyl)amino)-4-oxo-2-(pyridin-4-yl)-1,4,6,7-tetrahydro-5H-pyrrolo[3,2-c]pyridine-5-carboxylate, 7r

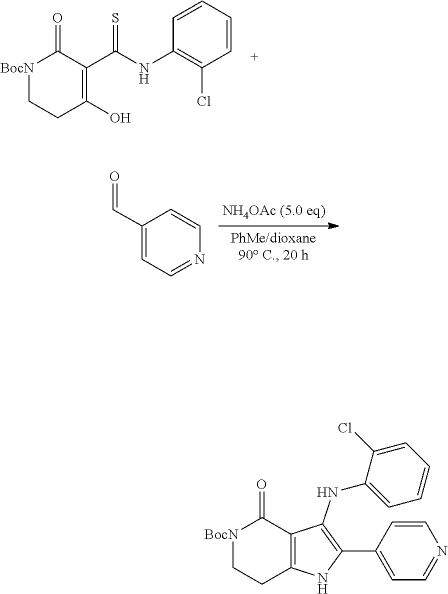 [SMILES: CC(C)(C)OC(=O)N1CCC(O)=C(C(=S)Nc2ccccc2Cl)C1=O ; O=Cc1ccncc1 ; CC(C)(C)OC(=O)N1CCc2[nH]c(-c3ccncc3)c(Nc3ccccc3Cl)c2C1=O]

Synthesised according to GP2 using 5b (50.0 mg, 0.131 mmol) and 6a (18.5 μL, 0.197 mmol). Purified by silica gel chromatography using a 12 g SiO₂ cartridge and a linear gradient of 0-100% EtOAc in heptane over 45 CV to give 7r (39.0 mg, 68%) as a yellow solid.

¹H NMR (400 MHz, Chloroform-d) δ 11.27 (s, 1H), 8.31 (d, J=5.4 Hz, 2H), 7.33 (d, J=5.4 Hz, 2H), 7.27 (d, J=4.7 Hz, 3H), 6.84 (t, J=7.7 Hz, 1H), 6.66 (t, J=7.6 Hz, 1H), 6.41 (d, J=8.1 Hz, 1H), 4.06 (t, J=6.4 Hz, 2H), 2.90 (t, J=6.3 Hz, 2H), 1.51 (s, 9H);

¹³C NMR (101 MHz, Chloroform-d) δ 163.8, 152.2, 144.0, 141.9, 141.7, 139.4, 129.5, 127.2, 121.6, 120.6, 119.7, 119.2, 114.4, 109.0, 83.5, 45.3, 28.2, 27.9, 22.5;

HRMS (ESI) calculated for C₂₃H₂₄ClN₄O₃⁺ [M+H]⁺ 439.1541; found m/z 439.1541.

### Example 39. Synthesis of tert-Butyl 3-((3-chlorophenyl)amino)-4-oxo-2-(pyridin-4-yl)-1,4,6,7-tetrahydro-5H-pyrrolo[3,2-c]pyridine-5-carboxylate, 7s

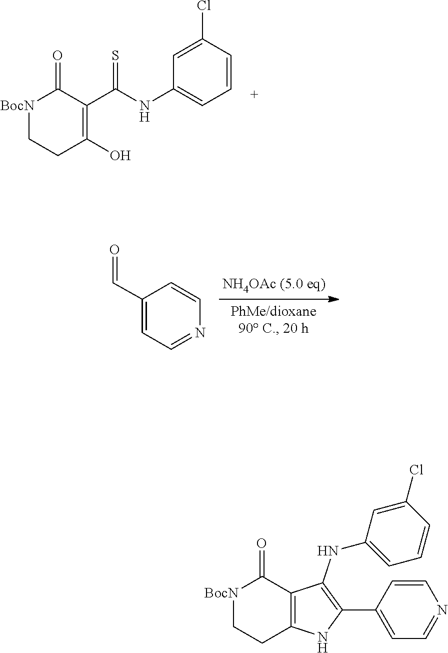 [SMILES: CC(C)(C)OC(=O)N1CCC(O)=C(C(=S)Nc2cccc(Cl)c2)C1=O ; O=Cc1ccncc1 ; CC(C)(C)OC(=O)N1CCc2[nH]c(-c3ccncc3)c(Nc3cccc(Cl)c3)c2C1=O]

Synthesised according to GP2 using 5s (50.0 mg, 0.131 mmol) and 6a (18.5 μL, 0.197 mmol). Purified by silica gel chromatography using a 12 g SiO₂ cartridge and a linear gradient of 0-100% EtOAc in heptane over 45 CV to give 7s (35 mg, 61%) as a yellow solid.

¹H NMR (400 MHz, Chloroform-d) δ 11.10 (s, 1H), 8.32 (d, J=6.3 Hz, 2H), 7.34 (d, J=6.3 Hz, 2H), 7.19 (s, 1H), 6.98 (t, J=8.0 Hz, 1H), 6.71 (d, J=7.9 Hz, 1H), 6.59-6.53 (m, 2H), 4.07 (t, J=6.3 Hz, 2H), 2.91 (t, J=6.3 Hz, 2H), 1.54 (s, 9H);

¹³C NMR (101 MHz, Chloroform-d) δ 164.3, 153.2, 148.1, 145.2, 140.3, 140.2, 135.1, 130.4, 129.3, 120.5, 119.5, 116.2, 114.7, 109.6, 83.7, 45.7, 28.8, 28.6, 23.2;

HRMS (ESI) calculated for C₂₃H₂₄ClN₄O₃⁺ [M+H]⁺ 439.1541; found m/z 439.1533.

### Example 40. Synthesis of tert-Butyl 3-((4-chlorophenyl)amino)-4-oxo-2-(pyridin-4-yl)-1,4,6,7-tetrahydro-5H-pyrrolo[3,2-c]pyridine-5-carboxylate, 7t

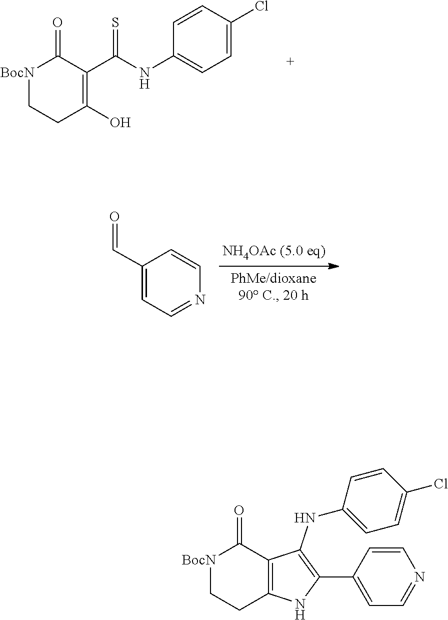 [SMILES: CC(C)(C)OC(=O)N1CCC(O)=C(C(=S)Nc2ccc(Cl)cc2)C1=O ; O=Cc1ccncc1 ; CC(C)(C)OC(=O)N1CCc2[nH]c(-c3ccncc3)c(Nc3ccc(Cl)cc3)c2C1=O]

Synthesised according to GP2 using 5t (50.0 mg, 0.131 mmol) and 6a (18.5 μL, 0.197 mmol). Purified by silica gel chromatography using a 12 g SiO₂ cartridge and a linear gradient of 0-100% EtOAc in heptane over 10 CV, followed by a linear gradient of 0-10% MeOH in DCM over 20 CV to give 7t (35 mg, 61%) as a yellow solid.

¹H NMR (400 MHz, Chloroform-d) δ 10.94 (s, 1H), 8.31 (d, J=6.1 Hz, 2H), 7.28 (d, J=6.1 Hz, 2H), 7.25 (s, 1H), 7.00 (d, J=8.8 Hz, 2H), 6.57 (d, J=8.8 Hz, 2H), 4.08 (t, J=6.3 Hz, 2H), 2.91 (t, J=6.3 Hz, 2H), 1.54 (s, 9H);

¹³C NMR (101 MHz, Chloroform-d) δ 163.9, 152.4, 143.9, 141.7, 141.6, 141.4, 131.5, 128.8, 125.1, 119.0, 118.1, 117.1, 108.6, 83.3, 45.1, 28.2, 27.9;

HRMS (ESI) calculated for C₂₃H₂₄ClN₄O₃⁺ [M+H]⁺ 439.1541; found m/z 439.1539.

### Example 41. Synthesis of tert-Butyl 3-((2-methoxyphenyl)amino)-4-oxo-2-(pyridin-4-yl)-1,4,6,7-tetrahydro-5H-pyrrolo[3,2-c]pyridine-5-carboxylate, 7u

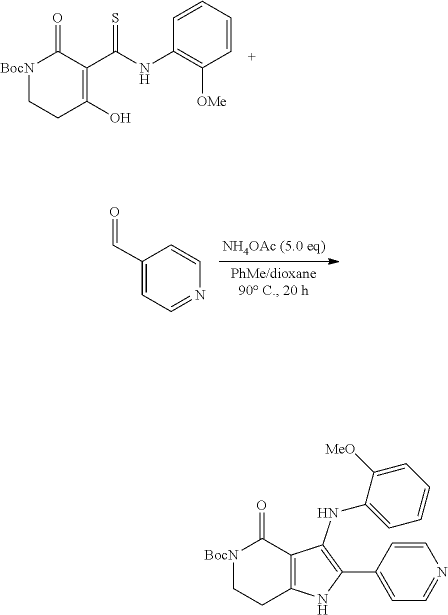 [SMILES: COc1ccccc1NC(=S)C1=C(O)CCN(C(=O)OC(C)(C)C)C1=O ; O=Cc1ccncc1 ; COc1ccccc1Nc1c(-c2ccncc2)[nH]c2c1C(=O)N(C(=O)OC(C)(C)C)CC2]

Synthesised according to GP2 using 5u (50.0 mg, 0.132 mmol) and 6a (18.7 μL, 0.198 mmol). Purified by silica gel chromatography using a 12 g SiO₂ cartridge and a linear gradient of 0-100% EtOAc in heptane over 40 CV to give 7u (37 mg, 64%) as a yellow solid.

¹H NMR (400 MHz, Chloroform-d) δ 10.56 (s, 1H), 8.31 (d, J=5.4 Hz, 2H), 7.47 (s, 1H), 7.32 (d, J=5.5 Hz, 2H), 6.81 (d, J=8.0 Hz, 1H), 6.70 (t, J=7.7 Hz, 1H), 6.54 (t, J=7.6 Hz, 1H), 6.26 (d, J=7.8 Hz, 1H), 4.05 (t, J=6.3 Hz, 2H), 3.86 (s, 3H), 2.89 (t, J=6.3 Hz, 2H), 1.51 (s, 9H);

¹³C NMR (101 MHz, Methanol-d4) δ 174.6, 163.0, 158.8, 158.5, 150.3, 150.1, 143.0, 139.0, 130.2, 129.5, 129.4, 129.1, 123.1, 120.4, 118.6, 93.0, 65.2, 55.8, 42.0, 37.3;

HRMS (ESI) calculated for C₂₄H₂₆N₄O₄⁺ [M+H]⁺ 435.2032; found m/z 435.2032.

### Example 42. Synthesis of tert-Butyl 3-((4-(ethoxycarbonyl)phenyl)amino)-4-oxo-2-(pyridin-4-yl)-1,4,6,7-tetrahydro-5H-pyrrolo[3,2-c]pyridine-5-carboxylate, 7v

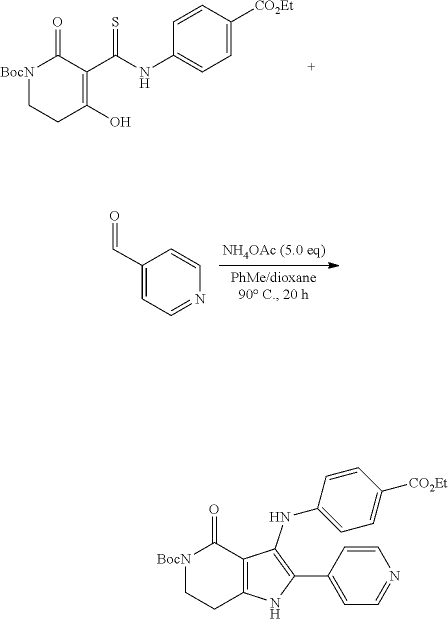 [SMILES: CCOC(=O)c1ccc(NC(=S)C2=C(O)CCN(C(=O)OC(C)(C)C)C2=O)cc1 ; O=Cc1ccncc1 ; CCOC(=O)c1ccc(Nc2c(-c3ccncc3)[nH]c3c2C(=O)N(C(=O)OC(C)(C)C)CC3)cc1]

Synthesised according to GP2 using 5v (50.0 mg, 0.119 mmol) and 6a (16.8 μL, 0.178 mmol). Purified by silica gel chromatography using a 12 g SiO₂ cartridge and a linear gradient of 0-100% EtOAc in heptane over 10 CV, followed by a linear gradient of 0-10% MeOH in DCM over 20 CV to give 7v (35 mg, 61%) as a yellow solid.

¹H NMR (400 MHz, Chloroform-d) δ 11.42 (s, 1H), 8.29 (d, J=6.0 Hz, 2H), 7.74 (d, J=8.5 Hz, 2H), 7.39 (s, 1H), 7.31 (d, J=6.0 Hz, 2H), 6.60 (d, J=8.5 Hz, 2H), 4.27 (q, J=7.1 Hz, 2H), 4.06 (t, J=6.3 Hz, 2H), 2.90 (t, J=6.3 Hz, 2H), 1.52 (s, 9H), 1.32 (t, J=7.1 Hz, 3H);

¹³C NMR (101 MHz, Chloroform-d) δ 167.2, 164.2, 153.1, 148.0, 139.6, 139.4, 131.4, 127.5, 121.7, 120.1, 119.5, 115.2, 109.6, 83.5, 61.0, 45.7, 28.8, 28.5, 23.2, 14.8;

HRMS (ESI) calculated for C₂₆H₂₉N₄O₅⁺ [M+H]⁺ 477.2138; found m/z 477.2138.

### Example 43. Synthesis of tert-Butyl 7-methyl-4-oxo-3-(phenylamino)-2-(pyridin-2-yl)-1,4,6,7-tetrahydro-5H-pyrrolo[3,2-c]pyridine-5-carboxylate, 7y

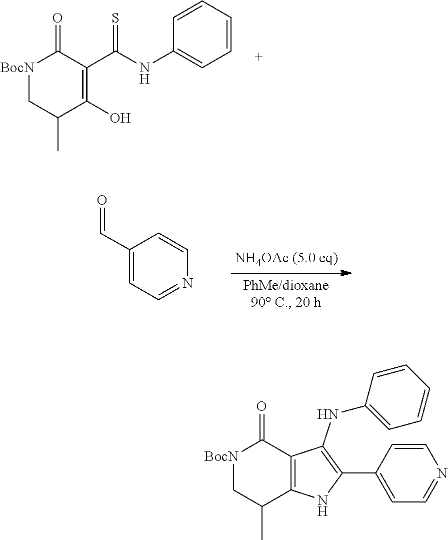 [SMILES: CC1CN(C(=O)OC(C)(C)C)C(=O)C(C(=S)Nc2ccccc2)=C1O ; O=Cc1ccncc1 ; CC1CN(C(=O)OC(C)(C)C)C(=O)c2c1[nH]c(-c1ccncc1)c2Nc1ccccc1]

Synthesised according to GP2 using 5y (100 mg, 0.287 mmol) and 6a (39.0 μL, 0.431 mmol). Purified by silica gel chromatography using a 24 g SiO₂ cartridge and a linear gradient of 20-100% EtOAc in heptane over 13 CV, followed by a linear gradient of 0-10% MeOH in DCM over 13 CV to give 7y (75 mg, 65%) as a yellow solid.

¹H NMR (400 MHz, Chloroform-d) δ 9.94 (br s, 1H), 8.30-8.24 (m, 2H), 7.37 (s, 1H), 7.28 (d, J=5.7 Hz, 2H), 7.06 (t, J=7.6 Hz, 2H), 6.76 (t, J=7.3 Hz, 1H), 6.65 (d, J=7.9 Hz, 2H), 4.15 (dd, J=13.1, 4.6 Hz, 1H), 3.70 (dd, J=13.0, 8.3 Hz, 1H), 3.26-3.13 (m, 1H), 1.55 (s, 9H), 1.37 (d, J=7.3 Hz, 3H);

¹³C NMR (101 MHz, Chloroform-d) δ 163.8, 153.0, 148.5, 143.2, 142.9, 139.1, 129.6, 128.8, 120.2, 118.9, 117.8, 116.3, 108.0, 83.0, 51.9, 28.5, 28.2, 15.9;

HRMS (ESI) calculated for C₂₄H₂₇N₄O₃⁺ [M+H]⁺ 419.2083; found m/z 419.2082.

### Example 45. Synthesis of 3-((3-fluoro-2-methoxyphenyl)amino)-2-(3-(2-methoxy-2-methylpropoxy)pyridin-4-yl)-1,5,6,7-tetrahydro-4H-pyrrolo[3,2-c]pyridin-4-one (18) 3-(2-Methoxy-2-methylpropoxy)isonicotinonitrile, 134

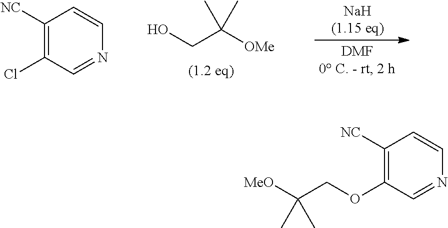

According to a modified literature procedure,⁴ 2-methoxy-2-methylpropan-1-ol (285 μL, 2.60 mmol, 1.20 eq) was added to a suspension of NaH (60% dispersion in mineral oil, 99.6 mg, 2.49 mmol, 1.15 eq) in DMF (6.00 mL) at 0° C. and the mixture stirred at that temperature for 15 minutes. 3-chloropyridine-4-carbonitrile (300 mg, 2.17 mmol, 1.00 eq) was added and the mixture stirred for 2 hours, allowing to warm to room temperature. The reaction mixture was quenched with water (60 mL) and extracted with EtOAc (3×30 mL). The combined organic extracts were washed with water and brine, dried over Na₂SO₄, filtered, and concentrated under reduced pressure. The residue was purified by silica gel chromatography using a 24 g SiO₂ cartridge and a linear gradient of 5-70% EtOAc in heptane to afford 13 (392 mg, 88%) as a white solid,

¹H NMR (400 MHz, Chloroform-d) δ 8.72 (s, 1H), 8.38 (d, J=4.8 Hz, 1H), 7.77 (dd, J=4.8, 0.7 Hz, 1H), 4.19 (s, 2H), 3.17 (s, 3H), 1.24 (s, 6H);

¹³C NMR (101 MHz, DMSO-d₆) δ 155.2, 142.7, 137.3, 126.5, 114.8, 108.3, 75.2, 74.3, 49.8, 22.3;

HRMS (ESI) calculated for C₁₁H₁₅N₂O₂⁺ [M+H]⁺ 207.1134; found m/z 207.1138.

The data matched those of the literature.⁴

### 3-(2-Methoxy-2-methylpropoxy)isonicotinaldehyde, 14

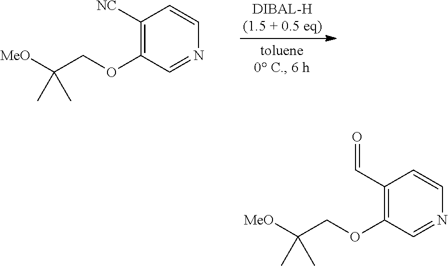

DIBAL-H (1.0 M in toluene, 2.18 mL, 0.728 mmol, 1.50 eq) was added dropwise to a solution of 13 (300 mg, 1.46 mmol, 1.00 eq) in toluene (15.0 mL) at 0° C. and the mixture stirred at that temperature for 4 hours. DIBAL-H (1.0 M in toluene, 727 μL, 0.728 mmol, 0.500 eq) was added and the mixture stirred for a further 2 hours. The mixture was quenched with MeOH and then diluted with 0.1 m HCl (50 mL). The mixture was extracted with DCM (3×40 mL) and the combined organic extracts were dried over Na₂SO₄, filtered, and concentrated under reduced pressure. The residue was purified by silica gel chromatography using a 24 g SiO₂ cartridge and a linear gradient of 0-6% MeOH in DCM to afford 14 (185 mg, 61%) as an orange solid.

¹H NMR (400 MHz, Chloroform-d) δ 10.58 (s, 1H), 8.55 (s, 1H), 8.40 (d, J=4.8 Hz, 1H), 7.59 (d, J=4.8 Hz, 1H), 4.06 (s, 2H), 3.29 (s, 3H), 1.34 (s, 6H);

¹³C NMR (101 MHz, Chloroform-d) δ 188.9, 155.3, 143.1, 137.2, 129.4, 119.9, 75.3, 74.2, 49.9, 22.1;

HRMS (ESI) calculated for C₁₁H₁₅NO₃⁺ [M+H]⁺ 210.1130; found m/z 210.1134.

### 1-Fluoro-3-isothiocyanato-2-methoxybenzene, 165

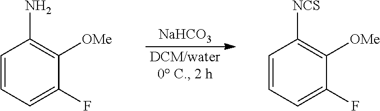

According to a literature procedure,⁵ thiophosgene (272 μL, 3.54 mmol, 1.00 eq) was added to a stirred mixture of 3-fluoro-2-methoxyaniline (500 mg, 3.54 mmol, 1.00 eq) in DCM (5.00 mL) and saturated aqueous NaHCO₃ (5.00 mL) and the mixture stirred at 0° C. for 2 hours. The layers were separated, and the aqueous layer extracted with DCM (2×10 mL). The combined organic extracts were dried over Na₂SO₄, filtered, and concentrated under reduced pressure to afford 16 (616 mg, 95%) as a brown oil that was used without further purification

¹H NMR (400 MHz, Chloroform-d) δ 7.09-6.79 (m, 3H), 4.03 (d, J=1.9 Hz, 3H);

¹⁹F NMR (376 MHz, Chloroform-d) δ −129.1.

The data matched those of the literature.⁵

### tert-Butyl 5-((3-fluoro-2-methoxyphenyl)carbamothioyl)-4-hydroxy-6-oxo-3,6-dihydropyridine-1(2H)-carboxylate, 175

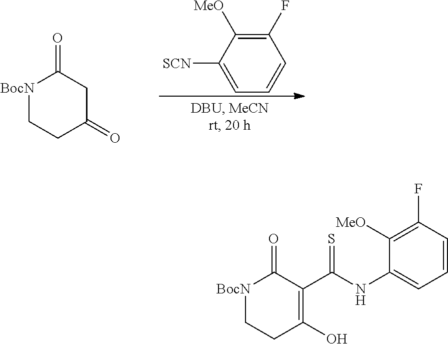

Synthesised according to GP1 using tert-butyl 2,4-dioxopiperidine-1-carboxylate (710 mg, 3.33 mmol) and 16 (610 mg, 3.33 mmol). Purified by silica gel chromatography using a 40 g SiO₂ cartridge and a linear gradient of 0-50% EtOAc in heptane over 15 CV to give 17 (960 mg, 73%) as an off-white solid.

¹H NMR (400 MHz, DMSO-d₆) δ 13.38 (s, 1H), 7.57 (dt, J=8.1, 1.5 Hz, 1H), 7.26 (ddd, J=11.3, 8.4, 1.6 Hz, 1H), 7.16 (td, J=8.3, 5.8 Hz, 1H), 3.86 (d, J=1.5 Hz, 3H), 3.79 (t, J=6.5 Hz, 2H), 2.89 (t, J=6.5 Hz, 2H), 1.49 (s, 9H);

¹³C NMR (101 MHz, DMSO-d₆) δ 189.9, 186.2, 155.6 (d, J=245.0 Hz), 152.3, 132.4 (d, J=4.3 Hz), 123.8 (d, J=8.7 Hz), 123.1 (d, J=3.1 Hz), 116.1 (d, J=18.9 Hz), 103.1, 83.1, 62.0 (d, J=4.9 Hz), 31.4, 28.1;

¹⁹F NMR (376 MHz, DMSO-d₆) δ −130.0;

HRMS (ESI) calculated for C₁₈H₂₀FN₂O₅S⁻ [M−H]⁻ 395.1077; found m/z 395.1077.

The data matched those of the literature.⁵

18⁴

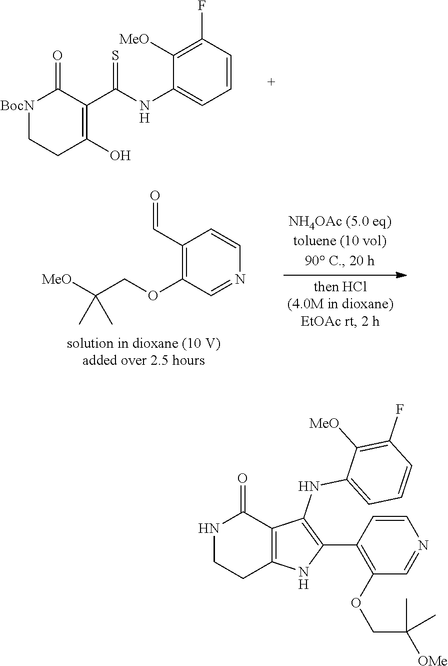 [SMILES: COc1c(F)cccc1NC(=S)C1=C(O)CCN(C(=O)OC(C)(C)C)C1=O ; COC(C)(C)COc1cnccc1C=O ; COc1c(F)cccc1Nc1c(-c2ccncc2OCC(C)(C)OC)[nH]c2c1C(=O)NCC2]

A solution of 14 (79.2 mg, 0.378 mmol, 1.50 eq) in 1,4-dioxane (1.00 mL) was added dropwise, over 2.5 hours using a syringe pump, to a mixture of 17 (100 mg, 0.252 mmol, 1.00 eq) and NH₄OAc (97.2 mg, 1.26 mmol, 5.00 eq) in toluene (1 mL) at 90° C. and the mixture stirred at 90° C. for 18 hours and then cooled to room temperature. The reaction mixture was concentrated to around 0.5 mL and EtOAc (1 mL) was added. HCl in 1,4-dioxane (4.0 m, 0.63 mL, 2.52 mmol, 10.0 eq) was added with vigorous stirring and the mixture stirred for 1 hour at room temperature. The reaction mixture was quenched with saturated aqueous NaHCO₃ (25 mL) and diluted with DCM (25 mL). The layers were separated, and the aqueous layer extracted with DCM (2×10 mL). The combined organic extracts were dried over Na₂SO₄, filtered, and concentrated under reduced pressure. The residue wasp purified by silica gel chromatography using a 24 g SiO₂ cartridge and a linear gradient of 20-100% EtOAc in heptane over 13 CV, followed by a linear gradient of 0-10% MeOH in DCM over 20 CV to give 18 (64 mg, 56%) as an off-white solid.

¹H NMR (400 MHz, DMSO-d₆) δ 11.17 (s, 1H), 8.42 (s, 1H), 8.02 (d, J=5.1 Hz, 1H), 7.51 (s, 1H), 7.29 (d, J=5.1 Hz, 1H), 7.16 (t, J=2.6 Hz, 1H), 6.67 (td, J=8.3, 6.1 Hz, 1H), 6.51 (ddd, J=11.0, 8.4, 1.5 Hz, 1H), 6.04 (dt, J=8.3, 1.3 Hz, 1H), 4.19 (s, 2H), 3.92 (d, J=0.8 Hz, 3H), 3.43 (td, J=6.9, 2.5 Hz, 2H), 3.26 (s, 3H), 2.84 (t, J=6.8 Hz, 2H), 1.28 (s, 6H);

¹³C NMR (101 MHz, DMSO-d₆) δ 165.8, 155.8 (d, J=242.0 Hz), 150.2, 143.3, 140.0 (d, J=4.7 Hz), 137.4, 136.1, 135.5 (d, J=13.4 Hz), 127.8, 125.9, 124.0 (d, J=9.8 Hz), 120.4, 117.1, 109.2, 108.1, 106.2 (d, J=19.0 Hz), 75.5, 75.1, 49.7, 40.5, 22.5, 22.1;

¹⁹F NMR (376 MHz, DMSO-d₆) δ −132.7;

HRMS (ESI) calculated for C₂₄H₂₈FN₄O₄ [M+H]⁺ 455.2095; found m/z 455.2098.

The data matched those of the literature.⁴

### Example 46. Scale-up synthesis of 2-(3-bromopyridin-4-yl)-3-((3-chloro-2-methoxyphenyl)amino)-1,5,6,7-tetrahydro-4H-pyrrolo[3,2-c]pyridin-4-one (Compound 102)

### Step 1:

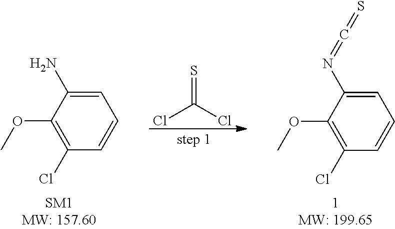

| | | | | Reaction | | | |
|---|---|---|---|---|---|---|---|
| | Starting Materials | | | Conditions | | IPC | |
| Lot | SM1 | | Reagents | Temp./Time | | Conversion | Result |
| 1 | 2 | g | SCCl₂: 1.3 eq. | 0~10 | C. | SM1: N.D | 2.0 g Oil |
| | 1.0 | eq. | NaHCO₃: 1.1 eq. | 4 | h | 1: 95.6 A % | Purity: 98.8 A % |
| | | | DCM/H₂O (4 V/4 V) | | | | Yield: 80% |
| | | | | | | | (uncorrected |
| | | | | | | | by QNMR) |
| 2 | 2 | g | SCCl₂: 1.05 eq. | 0~10 | C. | SM1: N.D | 24.5 g Oil |
| | 1.0 | eq. | TEA: 2.5 eq. | 3 | h | 1: 97.7 A % | Purity: 98.8 A % |
| | | | MTBE (25 V) | | | | Yield: 88% |
| 3 | 20 | g | SCCl₂: 1.05 eq. | 0~10 | C. | SM1: N.D | (corrected by |
| | 1.0 | eq. | TEA: 2.5 eq. | 3 | h | 1: 97.4 A % | QNMR) |
| | | | MTBE (25 V) | | | | |

| | | | | Reaction | | IPC | |
|---|---|---|---|---|---|---|---|
| | Starting Materials | | | Conditions | | SM1: 5.64 min | |
| Lot | SM1 | | Reagents | Temp./Time | | Compound 1: 9.28 min | Result |
| 4 | 50 | g | SCCl₂: 1.05 eq. | 0~10 | C. | 5.64 min: 1.6% | 63.9 g Oil |
| | 1.0 | eq. | TEA: 2.5 eq. | 3 | h | 9.28 min: 97.6% | Purity: 96.4 A % |

| | | |
|---|---|---|
| QNMR: 99% | MTBE (25 V) | Yield: 86% |
| | | (corrected by |
| | | QNMR) |

| | | | | Reaction | | IPC | |
|---|---|---|---|---|---|---|---|
| | Starting Materials | | | Conditions | | SM1: 6.12 min | |
| Lot | SM1 | | Reagents | Temp./Time | | Compound 1: 9.65 min | Result |
| 5 | Each 1 | g | SCCl₂: 1.05 eq. | 0~10 | C. | The reaction | N/A |
| | 1.0 | eq. | TEA: 2.5 eq. | 3 | h | mixture is very | |

| | QNMR: 99% | MTBE (10 V) | | | sticky | |
|---|---|---|---|---|---|---|
| 6 | | SCCl₂: 1.05 eq. | 0~10 | C. | 6.12 min: 0.3% | Sticky as well. |
| | | TEA: 2.5 eq. | 3 | h | 9.65 min: 97.9% | |
| | | IPAC (10 V) | | | | |
| 7 | | SCCl₂: 1.05 eq. | 0~10 | C. | 6.12 min: 4.5% | Solid precipitate out. |
| | | TEA: 2.5 eq. | 3 | h | 9.65 min: 86.4% | |
| | | DCM (10 V) | | | | |
| 8 | | SCCl₂: 1.05 eq. | 0~10 | C. | 6.12 min: 9.5% | Solid precipitate out. |
| | | TEA: 2.5 eq. | 3 | h | 9.65 min: 75.3% | |

ACN (10 V)

10 V of IPAc provided highest WC purity (97.9 A %).

| | | | Reaction | | |
|---|---|---|---|---|---|
| | Starting Materials | | Conditions | | |
| Lot | SM1 | Reagents | Temp./Time | IPC | Result |
| 9 | Each | SCCl₂: 1.05 eq. | 0~10 C. | SM1 (5.64 min): | TEA salt |
| | 5 g | TEA: 2.5 eq. | 3 h | 1.6% | precipitated |
| | 1.0 eq. | MTBE (25 V) | | Compound 1 (9.28 | out. |
| | QNMR: | | | min): 97.6% | |
| 10 | 99% | SCCl₂: 1.05 eq. | 0~10 C. | SM1 (6.33 min): | DIEA salt |
| | | DIEA: 2.5 eq. | 3 h | 0.1% | precipitated |
| | | MTBE (25 V) | | Compound 1 (9.73 | out. |
| | | | | min): 98.2% | |
| 11 | | SCCl₂: 1.05 eq. | 0~10 C. | SM1 (6.31 min): | CSC12 may |
| | | DBU: 2.5 eq. | 3 h | 32.8% | be |
| | | MTBE (25 V) | | Compound 1 (9.72 | consumed |
| | | | | min): 42.8% | by DBU. |

Different base (TEA, DIIEA and DBU) were compared on 5 g scale reactions, DIEA provided 98.2 A % IPC purity.

| | | | Reaction | | |
|---|---|---|---|---|---|
| | Starting Materials | | Conditions | | |
| Lot | SM1 | Reagents | Temp./Time | IPC | Result |
| 12 | 20 g | SCCl₂: 1.05 eq. | 0~10 C. | SM1 (6.33 | 371 g of IPAc's solution |
| | 1.0 eq. | DIEA: 2.5 eq. | 3 h | min): ND | QNMR: 6.2% |
| | QNMR: 99% | | | Compound | P:94.4 A % |
| | | IPAC (20 V) | | 1 (9.66 | Y: 90% by QNMR |
| | | | | min): 98.2% | KF: 1.3% |
| 13 | 20 g | SCCl₂: 1.0 eq. | 0~10 C. | SM1 (5.71 | 116 g of IPAc's solution |
| | 1.0 eq. | DIEA: 2.5 eq. | 3 h | min): 1.1% | QNMR: 19.4% |
| | QNMR: 99% | IPAC (20 V) | | Compound | P:96.7A % |
| | | | | 1 (9.25 | Y:88.7% by QNMR |
| | | | | min): 98.5% | KF:0.16% |

### Step 2:

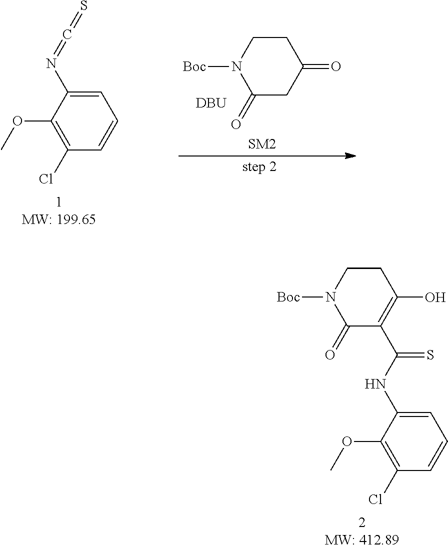

| | | | Reaction | | |
|---|---|---|---|---|---|
| | Starting Materials | | Conditions | IPC | |
| Lot | 1 | Reagents | Temp./Time | Conversion | Result |
| 1 | 2 g | SM2: 1.0 eq. | 0~10 C. | 1: N.D | 38.6 g |
| | 1.0 | DBU: 1.5eq. | 3 h | 2: 85.4 A % | Purity: 96.1 A % |
| | eq. | ACN (6.5 V) | | | Yield: 85% |
| 2 | 20 g | SM2: 1.0 eq. | 0~10 C. | 1: 0.7 A % | (uncorrected by |
| | 1.0 | DBU: 1.5eq. | 3 h | 2: 90.8 A % | QNMR) |
| | eq. | ACN (6.5 V) | | | |

| | | | | | IPC | |
|---|---|---|---|---|---|---|
| | Starting Materials | | | Reaction | 1: ~9.62 min | |
| | Compound | | | Conditions | Compound | |
| Lot | 1 | SM2 | Reagents | Temp./Time | 2: ~10.15 min | Result |
| 3 | 10 g | 1.0 eq. | DBU: | 0~25 C. | 9.62 min: 0.6 A % | 15 g |
| | 1.0 eq. | Assay: 99% | 1.5eq. | 12 h | 10.15 min: 79.7 A % | 99.8 A % |
| | P: 96.4 A % | | ACN (10 V) | | | Y: 75% |
| | Corrected | | | | | |
| | by QNMR | | | | | |
| 4 | 1 g | 1.0 eq. | DBU: | 0~25 C. | 9.65 min: 0.4 A % | Sticky. |
| | 1.0 eq. | Assay: 99% | 1.5eq. | 12 h | 10.17 min: 85.9 A % | |
| | P: 96.4 A % | | IPAC (10 | | | |
| | Corrected | | V) | | | |
| | by QNMR | | | | | |
| 5 | 1 g | 1.0 eq. | DBU: | 0~25 C. | 9.62 min: 14.6 A % | N/A |
| | 1.0 eq. | Assay: 99% | 1.5eq. | 12 h | 10.15 min: 51.5 A % | |
| | P: 96.4 A % | | DCM (10 | | | |
| | Corrected | | V) | | | |
| | by QNMR | | | | | |

| | | | | Reaction | | |
|---|---|---|---|---|---|---|
| | Starting Materials | | | Conditions | | |
| Lot | 1 | SM2 | Reagents | Temp./Time | IPC | Result |
| 6 | Each 5 g | 1.0 eq. | DBU: 1.1eq. | 0~25 C. | Compound 1 (9.62 | Heterogeneous |
| | 1.0 eq. | Assay: 99% | IPAC (20 V) | 12 h | min): 0.4 A % | |
| | | | | | Compound 2(10.15 | |
| | | | | | min): 9 | |
| | | | | | 0.4 A % | |
| 7 | P: 96.4 | 1.0 eq. | DBU: 0.2 eq. | 0~25 C. | Compound 1 (9.59 | Homogeneous |
| | | Assay: 99% | IPAC (20 V) | 12 h | min): 77.8 A % | |
| | | | | | Compound 2 (10.15 | |
| | | | | | min): 4.1 A % | |
| 8 | A % | 1.0 eq. | IPAC (20 V) | 0~25 C. | No reaction | Homogeneous |
| | | Assay: 99% | | 12 h | | |
| 9 | Corrected | 1.0 eq. | DIEA: 2.0 eq. | 0~25 C. | 1 (9.62 min): 72.7 | Homogeneous |
| | | | | 12 h | A % | |
| | | | | | Compound 2 (10.15 | |
| | | | | | min): 6.9 A % | |
| | by | Assay: 99% | IPAC (20 V) | 40-50 C. | 1 (9.24 min): 52.3 | |
| | | | | 12 h | A % | |
| | | | | | Compound 2 (9.8 | |
| | | | | | min): 18.6 A % | |
| 10 | QNMR | 1.0 eq. | TEA: 2.0 eq. | 40-50 C. | 1 (9.24 min): 48.7 | Homogeneous |
| | | Assay: 99% | IPAC (20 V) | 12 h | A % | |
| | | | | | 1 (9.8 min): 33.3 | |
| | | | | | A % | |

Different bases (DBU, DIEA and TEA), equivalents of base, and temperature were screened using 20 V of IPAc as solvent, 1.1 equivalents of DBU provided 90.4 A % IPC purity.

| | | | | Reaction | | |
|---|---|---|---|---|---|---|
| | Starting Materials | | | Conditions | | |
| Lot | 1 | SM2 | Reagents | Temp./Time | IPC | Result |
| 11 | 2 g | 1.0 eq. | DBU: 1.1 eq. | 0~25 C. | 1 (9.67 | N/A. |
| | 1.0 eq. | Assay: 99% | IPAC (20 V) | 12 h | min): 53.3 | |
| | P: 96.4 A % | | | | A % | |
| | Corrected by | | | | Compound 2 | |
| | QNMR | | | | (10.18 min): | |
| | KF: 0.2% by | | | | 34.5 A % | |
| | add H₂O | | | | | |
| 12 | 2 g | 1.0 eq. | DBU: 1.1 eq. | 0~25 C. | 1 (9.65 | N/A |
| | 1.0 eq. | Assay: 99% | | 12 h | min): 45.5 | |
| | 29.8 g IPAC | | | | A % | |
| | solution | | | | Compound 2 | |
| | KF: 1.3% | | | | (10.17 min): | |
| | P: 94.4 A % | | | | 45.3 A % | |
| 13 | 2 g | 1.0 eq. | DBU: 1.1 eq. | 0~25 C. | Compound 1 | N/A |
| | 1.0 eq. | Assay: 99% | IPAC (14 V) | 12 h | (9.65 min): | |
| | 10.3 g IPAC | | | | 2.7 A % | |
| | solution | | | | Compound 2 | |
| | KF: 0.16% | | | | (10.17 min): | |
| | P: 96.7 A % | | | | 87.8 A % | |

Different bases (DBU, DIEA and TEA), equivalents of base, and temperature were screened using 20 V of IPAc as solvent, 1.1 equivalents of DBU provided 90.4 A % IPC purity. This condition was used for scale up.

Solubility data of Compound 2 & Compound 2 containing DBU

| | | Solubility Compound 2 |
|---|---|---|
| Solvent | Solubility Compound 2 | containing DBU |

| | | | | |
|---|---|---|---|---|
| n-heptane | <5 | mg/ml | <5 | mg/ml |
| EtOAc | 100 | mg/ml | <5 | mg/ml |
| DCM | >200 | mg/ml | >200 | mg/ml |
| MeOH | 5 | mg/ml | 50 | mg/ml |
| EtOH | <5 | mg/ml | 25 | mg/ml |
| MTBE | 28 | mg/ml | <5 | mg/ml |
| ACN | 33 | mg/ml | 33 | mg/ml |

### Step 1&2: (Telescope Procedure)

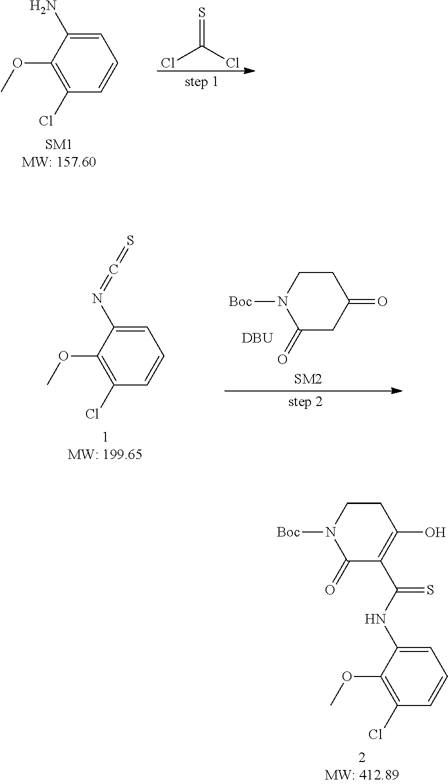

| | | | Reaction | | | | Reaction | | |
|---|---|---|---|---|---|---|---|---|---|
| | Step 1 | | Conditions | | | | Conditions | | |
| | Starting Materials | | Temp./ | | | Step 2 | Temp./ | | |
| Option | SM1 | Reagents | Time | IPC | Work up | Reagents | Time | IPC | Result |
| 1 | Each 100 g | SCCl₂: | 0~10 C. | SM1: | Filtrate | SM2: | 0~25 C. | 1: 1.1 A % | 204 g |
| | 1.0 eq. | 1.0 eq. | 3 h | 1.1 A % | Use the | 1.0 eq. | 12 h | 2: 91.0 A % | P: 99.8% |
| | QNMR: 99% | DIEA: | | 1: 97.4 | filtrate to | DBU: | | | Y: 78% |
| | | 2.5 eq. | | A % | next step | 1.0 eq. | | | (2 step) |
| | | IPAC | | | directly | | | | |
| | | (20 V) | | | (KF: 0.032%) | | | | |
| 2 | | | | SM1: | Quench with | | | 1: 0.7 A % | 205 g |
| | | | | 0.9 A % | 5% aq. | | | 2: 89.3 A % | P: 99.4% |
| | | | | 1: 98.2 | NaHCO₃ (10 | | | | Y: 78% |
| | | | | A % | V) | | | | (2 step) |
| | | | | | Concentrate | | | | |
| | | | | | to KF ≤ 0.1% | | | | |

| | | 0 d | 1 d | 2 d | 3 d |
|---|---|---|---|---|---|
| Quenching the | 20 ± 5 C. | 96.9 A % | 94.5 A % | 94.9 A % | N/A |
| reaction of step | | | | | |
| 1 with | | | | | |
| aq.NaHCO3 | | | | | |
| Comp. 1's IPAC | 40 ± 5 C. | | 96.0 A % | 95.9 A % | 94.3 A % |
| solution | 50 ± 5 C. | | 95.5 A % | 94.7 A % | 90.5 A % |
| Comp. 2's | 20 ± 5 C. | 90.1 A % | 89.8 A % | N/A | 90.9 A % |
| IPAC solution | | | | | |
| pH = 5-6 | | | | | |
| Comp. 2's | 25 ± 5 C. | 87.3% | 88.7% | 86.5% | 85.2% |
| IPAC solution | 25 ± 5 C. | | 86.0% | 85.6% | 83.5% |
| | 40 ± 5 C. | 90.1 A % | 88.6 A % | N/A | 84.7 A % |
| | 40 ± 5 C. | | 87.7 A % | N/A | 80.0% |
| Comp. 2 on | 45 ± 5 C. | 99.7 A % | N/A | N/A | 99.8% |
| drying | | | | | |

### Step 3:

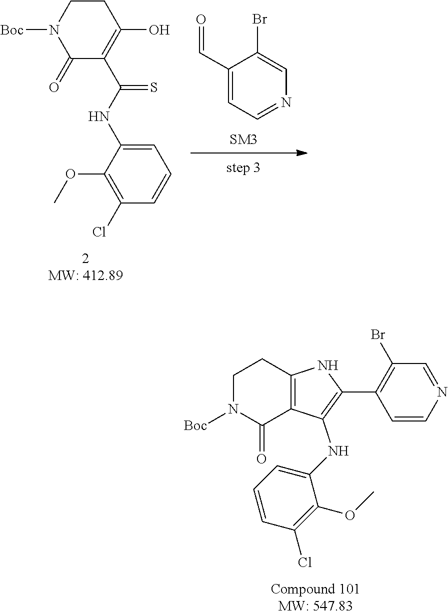

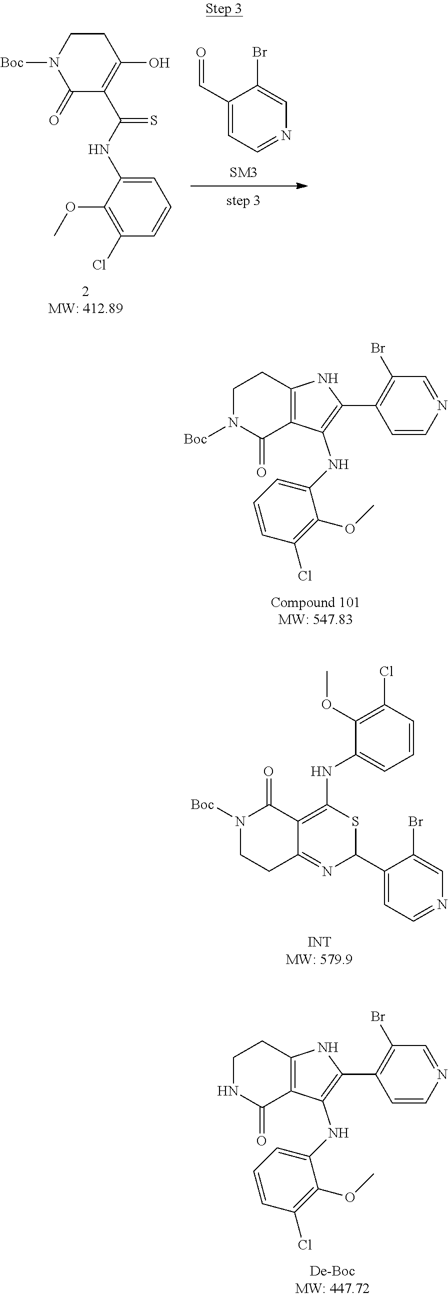

| | | | | IPC | |
|---|---|---|---|---|---|
| | | | | Compound 2: 22.4 | |
| | Starting Materials | | Reaction | min | |
| | Compound | | Conditions | Compound 101: 17.3 | |
| Lot | 2 | Reagents | Temp./Time | min | Result |
| 1 | 1 g | SM3: 1.5 eq. | 95 = 5 C. | 22. 4 min: 0.2 A % | 0.5 g |
| | P: 99.8 A % | NH₄OAc: 5.0 | 16 h | 17. 3 min: 56.9 A % | 97.1 A % |
| | Assay:98% | eq. | | | Y: 38% |
| | 1.0 eq. | toluene: 15 V | | | |
| | | Dioxane: 7.5 V | | | |
| 2 | 5 g | SM3: 1.5 eq. | 95 = 5 C. | 22.4 min: 0.8 A % | 3.83 g of |
| | P: 99.8 A % | NH₄OAc: 5.0 | 16 h | 17.3 min: 57.8 A % | Compound |
| | Assay:98% | eq. | | | 101 in the |
| | 1.0 eq. | toluene: 15 V | | | reaction |
| | | Dioxane: 7.5 V | | | mixture by |
| | | | | | assay |
| | | | | | Y: 57.8% |

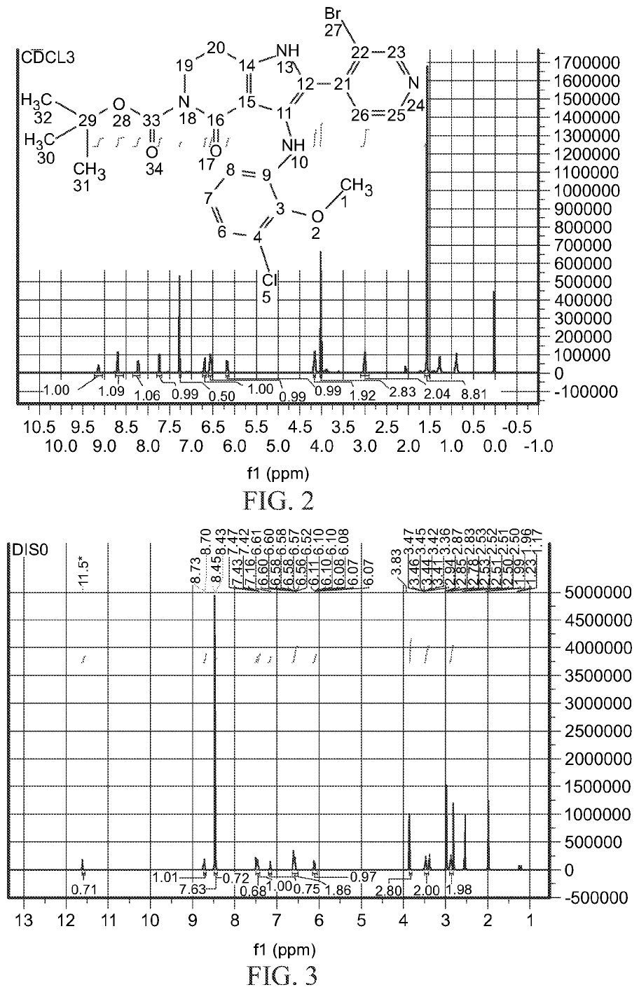

HNMR Data of Compound 101 is included in FIG. 2.

Solubility data of Compound 101

| Solvent | Solubility | |
|---|---|---|
| n-heptane | <5 | mg/ml |
| EtOAc | 6.25 | mg/ml |
| DCM | >125 | mg/ml |
| MeOH | 7.1 | mg/ml |
| EtOH | 6.25 | mg/ml |
| MTBE | 6.25 | mg/ml |
| ACN | 33 | mg/ml |
| toluene | <5 | mg/ml |

| | | | | IPC | |
|---|---|---|---|---|---|
| | | | | Compound 2: 22.4 | |
| | Starting Materials | | Reaction | min | |
| | Compound | | Conditions | Compound 101: | |
| Lot | 2 | Reagents | Temp./Time | 17.3 min | Result |
| 3 | 30 g | SM3: 1.5 eq. | 95 = 5 C. | 22.4 min: 1.0 A % | Will be used to |
| | P: 99.8 | NH4OAc: | 16 h | 17.3 min: 52.3 | next step directly |
| | A % | 5.0 eq. | | A % | |
| | Assay:98% | toluene: 15 | | De-Boc: 5.5 A % | |
| | 1.0 eq. | V | | INT: 0.5 A % | |
| | | Dioxane: 7.5 | | Assay yield: | |
| | | V | | 54.3% | |

### Step 3&4

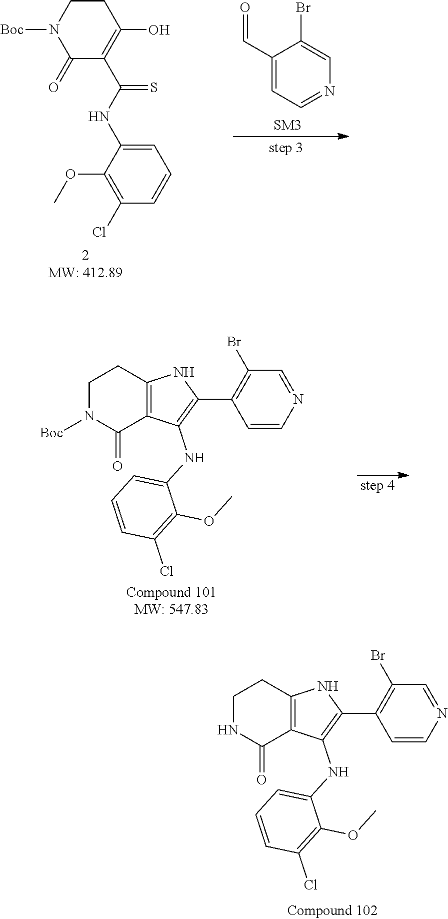 [SMILES: COc1c(Cl)cccc1NC(=S)C1=C(O)CCN(C(=O)OC(C)(C)C)C1=O ; O=Cc1ccncc1Br ; COc1c(Cl)cccc1Nc1c(-c2ccncc2Br)[nH]c2c1C(=O)N(C(=O)OC(C)(C)C)CC2 ; COc1c(Cl)cccc1Nc1c(-c2ccncc2Br)[nH]c2c1C(=O)NCC2]

| | | | | | Step 4: | |
|---|---|---|---|---|---|---|
| | Starting Materials | | Reaction | | Reaction | |
| | Solution | Solution | Conditions | | Conditions | |
| Lot | 1 | 2 | Temp./Time | IPC | Temp./Time | IPC |
| 1 | 2: 20 g | SM3: 1.5 eq | Solution | 2 (22.4 min): 10.0A % | N/A | N/A |
| | (1.0 eq.) | toluene: | 2addedto | Compound 101 | | |
| | NH₄OAc: | 7.5 V | solution 1 | (17.3 min): 36.4 A % | | |
| | 5.0 eq. | Dioxane: | with Dean- | Compound 102 | | |
| | toluene: | 7.5 V | stark trap and | (11.24 min): 21.4 | | |
| | 7.5 V | | reflux for 3 h, | A % | | |
| | | | stir with | INT: N.D | | |
| | | | reflux for 16 h | | | |
| | | 0.2 eq. | Re-charged | 2 (22.4 min): 3.4 | Add 60 mL | Compound |
| | | SM3: in | 1.5 eq. | A %; Compound 101 | HCl/MeOH | 101: |
| | | 1 V | NH₄OAc to | (17.3 min): 12.8 A %, | (4M) | 17.6 min: |
| | | toluene | solution 1. | Compound 102 | Stir 2 h at | ND |
| | | and | Solution 2 | (11.24min): 49.0 | 20 ± 5 C. | Compound |
| | | Dioxane | added to | A %, INT: N.D | | 102: |
| | | | solution 1 | | | 11.2 min: |
| | | | with Dean- | | | 62.7 A % |
| | | | stark trap and | | | Assay |
| | | | reflux for 1 h, | | | yield: |
| | | | stir with | | | 60.4% |
| | | | reflux for 16 h | | | |

| | | | | | Step 4: | |
|---|---|---|---|---|---|---|
| | Step 3: Starting | | Reaction | | Reaction | |
| | Materials | | Conditions | | Conditions | |
| Lot | 2 | Reagents | Temp./Time | IPC | Temp./Time | IPC |
| 2 | 50 g | SM3: 1.5eq; | 95 ± 5 C.; | 2: 22.4 min: | Add | Compound |
| | P: | NH₄OAc: 5.0eq. | 16 h | 2.0A % | 150 mL | 101: 17.3 |
| | 99.8 | toluene: 15 V | | Compound | HCl/MeOH | min: |
| | A % | Dioxane: 7.5 V | | 101: 17.3 min: | (4M) | ND |
| | Assay | | | 56.1 A % | Stir 2 h at | Compound |
| | 98%; | | | Compound 102: | 20 ± 5 C. | 102: 11.2 |
| | 1.0 | | | 11.2 min: 4.2 | | min: |
| | eq. | | | A % | | 61.2 A % |
| | | | | INT: 0.2 A % | | |

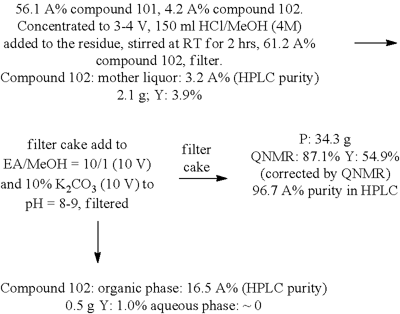

| Compound 102 | Conditions | | result |
|---|---|---|---|
| Each 5 g | DCM (5 V) with | Drying at | 4.8 g; P: 96.1 A % |
| P: 95.3 A % | reflux for 2 h | 60 C. for 24 h | Residual S: 0.20% by IC |
| | (suspension) | | KF :2.0% |
| | MeCN (5 V) with | | 4.5 g; P: 96.6 A % |
| | reflux for 2 h | | Residual S:0.17% by IC |
| | (suspension) | | KF: 0.18% |
| | MTBE (5 V) with | | 4.9 g; P: 95.7 A % |
| | reflux for 2 h | | Residual S:0.25% by IC |
| | (suspension) | | KF:0.19% |
| | THF (5 V) with reflux | | 2.0 g; P: 98.6 A % |
| | for 2 h | | KF: 2.0% |
| | (clear solution) | | |
| | MeOH (5 V) with | | 2.5 g; P: 98.0 A % |
| | reflux for 2 h | | KF: 1.8% |
| | (suspension) | | |
| 5 g | EtOAc (5 V) with | | 4.8 g; P: 96.9 A % |
| P: 96.7 A % | reflux for 2 h | | KF: 1.8% |
| QNMR:87.1 A % | (suspension) | | |
| Residual S: 1.0% | Toluene (5 V) with | | 4.0 g; P: 97.1 A % |
| w/w by IC | reflux for 16h | | QNMR: 95.7 A % |
| KF: 2.0% | (suspension) | | Residual S:0.056% by IC |
| | | | KF: 0.081% |

The crystallization for purification of Compound 102 with 5 V of different solvents (DCM, MeCN, MTBE, TIE, MeOH, EtOAc and toluene) were tried, however, Compound 102 has not been dissolved in all these solvents at reflux except for THE.
THF as solvent for purification, the purity of Compound 102 increased to 98.6 A % from 95.3 A %, the yield was about 40%.
Toluene as solvent for purification, the residual S reduced to 0.056% w/w from 1.0% w/w by IC and the QNMR increased to 95.7% from 87.1%, and HPLC purity 97.1 A).

Solubility data of Compound 102

| Solvent | Solubility / mg/ml |
|---|---|
| DMF | >100 |
| NMP | >100 |
| DMAC | >200 |
| DMSO | >100 |
| HFIP | 94 |
| TFE | 79 |
| THE | 31 |
| 2-MeTHF | 5 |
| MeCN | 2 |
| Tol | ~0 |

| | | | | | Step 4: | |
|---|---|---|---|---|---|---|
| | | | Reaction | | Reaction | |
| | Step 3: Starting Materials | | Conditions | | Conditions | |
| Lot | Solution 1 | Solution 2 | Temp./Time | IPC | Temp./Time | IPC |
| 3 | 2: 50 g (1.0 eq.) | SM3: | Solution 2 | 2: 22.4 min: | Add 150 mL | Compound |
| | NH₄OAc: 3.0 | 1.5 cq. | added to | 4.9 A% | HCI/McOH | 101: 17.4 |
| | eq. | toluene: | solution 1 | Compound | (4M) | min: ND |
| | toluene: 7.5 V | 15 V | at 95 ± 5 C. | 101: 17.4 | Stir 2 h at | Compound |
| | H₂O (0.2 V) | Dioxane: | for 6 h, stir | min: 60.7 | 20 ± 5 C | 102: 11.2 |
| | | 7.5 V | at 95 ± 5 for | A% | | min: 58.6 |
| | | | 16 h | Compound | | A% |
| | | | | 102: 11.2 | | Assay |
| | | | | min: 2.4 A% | | yicld: 60.2% |
| | | | | INT: 3.0 A% | | |
| | | | | Assay yield | | |
| | | | | (Compound | | |
| | | | | 101): 64.4% | | |

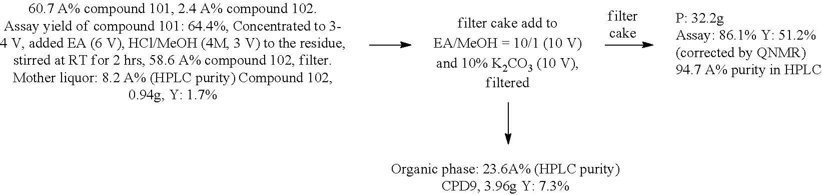

| | | | | | | |
|---|---|---|---|---|---|---|
| 4 | 2: 50 g | SM3: 1.5 | Solution 2 | 2: 22.4 | Add 150 mL | Compound |
| | (1.0 eq.) | eq. | added to | min: 2.7 | HCl/MeOH | 101: 17.4 |
| | NH₄OAc: | toluene: 15 | solution 1 | A% | (4M) | min: 0.3 |
| | 5.0 eq. | V | at 95 ± 5 C. | Compound | Stir 2 h at | A% |
| | toluene: | Dioxane: | for 6 h, stir | 101: 17.4 | 20 ± 5 C. | Compound |
| | 7.5 V | 7.5 V | at 95 ± 5 for | min: 59.8 | | 102: 11.2 |
| | H₂O (0.2 | | 16 h | A% | | min: 59.5 |
| | V) | | | Compound | | A% |
| | | | | 102: 11.2 | | Assay |
| | | | | min: 3.9 | | yield:65.1% |
| | | | | A% | | |
| | | | | INT: 1.8 | | |
| | | | | A% | | |
| | | | | Assay yield | | |
| | | | | (Compound | | |
| | | | | 101): | | |
| | | | | 65.2% | | |

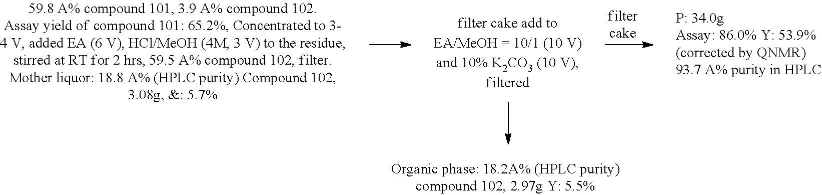

| Compound 102 | Conditions | Result |
|---|---|---|
| 5 g | THF (7 V) with reflux for 2 h (clear | 3.6 g |
| P: 95.2 A % | solution). | P: 97.6 A % |
| QNMR: 88% | Charge 7 V of toluene drop-wise to the | Yield: 72% |
| Residual S: | solution at 65+5C. | QNMR: 96.7% |
| 2.24% | Stir for at least 1 h at 65+5C. | Residual S: 0.15% |
| | Cool to 20 ± 5 C. and stir for at least 1 h. | |
| | Filtrate. | |
| Each 500 mg | NMP (3 V) with R.T for 1 h. (clear solution) | P: 98.8 A % |
| P: 95.2 A % | Charge 7 V of toluene to the solution. | (NMP are not integrated) |
| QNMR: 88% | (suspension) | |
| Residual S: | Stir for at least 1 h at R.T. | Residual S: 0.53% |
| 2.24% | DMAc (3 V) with R.T for 1 h. (clear | P: 99.2 A % |
| | solution) | (DMAC are not integrated) |
| | Charge 10 V of toluene to the solution. | Residual S: 0.37% |
| | (suspension) | |
| | Stir for at least 1 h at R.T. | |
| | NMP (3 V) with R. T for 1 h. (clear solution) | P: 97.6 A % |
| | Charge 3 V of water to the solution. | (NMP are not integrated) |
| | (suspension) | Residual S: 0.10% |
| | Stir for at least 1 h at R.T. | |
| | DMAc (3 V) with R.T for 1 h. (clear | P: 97.8 A % |
| | solution) | (DMAC are not integrated) |
| | Charge 3 V of water to the solution. | Residual S: 0.10% |
| | (suspension) | |
| | Stir for at least 1 h at R.T. | |
| | EtOH (10 V) with reflux for 2 h. | P: 97.3 A % |
| | (suspension) | Residual S: 0.5% |
| | Cool to R. T and stir for at least 2 h. | |
| 5 g | DMAc (2 V) with R.T for 1 h. (clear | 3.6 g |
| P: 95.2 A % | solution) | P: 99.1 A % |
| QNMR: 88% | Charge 10 V of toluene to the solution. | (DMAC are not integrated) |
| Residual S: | (suspension) | Yield: 72% |
| 2.24% | Stir for at least 1 h at R.T. | QNMR: 88% (12% DMAc residual |
| | Filtrate. | by QNMR) |
| | | Residual S: 0.35% |

To upgrade the purity of Compound 102 and reduce the content of sulfur in Compound 102, the crystallization/slurry with different solvents were tried, DMAc/toluene condition gave the best result for purity upgrade (from 95.2 A % to 99.2 A %), NMP/water and DMAc/water conditions gave the best S removal capacity (from 2.24% to 0.10%).
5 g of crude Compound 102 was purified using DMAc/toluene condition. 3.6 g of Compound 102 was obtained with 99.1 A % purity (DMAC are not integrated) and 72% yield (uncorrected by QNMR). The residual S was 0.35% and the potency by QNMR was 88%.

¹H NMR of compound 102 is included in FIG. 3. Around 1200 of DMAC was remained in Compound 102.

| Compound 102 | Conditions | Result |
|---|---|---|
| 3.5 g | Water (10 V) at 45 ± 5° C. for 1 h. | 2.7 g |
| P: 99.1 A % (DMAC | (suspension) | P: 99.2 A % |
| are not integrated) | Cool to 25 ± 5 C. and stir for 1 h. | Yield: 77% |
| QNMR: 88% | Filtrate. | QNMR: 99.5% |
| Residual S: 0.35% | | Residual S: <LOQ |
| | | (161 ppm) |
| 500 mg | DMAC (2 V) and toluene (2 V) at | P: 99.2 A % |
| P: 95.2 A % | 75 ± 5° C. for 1 h. (clear solution) | |
| QNMR: 88% | Cool to 45 = 5 C and charge toluene (5 | |
| Residual S: 2.24% | V) drop-wise to the mixture. | |
| | (suspension) | |
| | Stir for 1 h at 45 ± 5 C. and cool to | |
| | 25 ± 5 C. (suspension) | |
| | Stir for at least 1 h. (suspension) | |
| 10 g | DMAC (2 V) and toluene (2 V) at | 8.5 g |
| P: 95.2 A % | 75 ± 5° C. for 1 h. (clear solution) | P: 99.2 A % |
| QNMR: 88% | Cool to 45 ± 5 C. and charge toluene (5 | Yield: 85% |
| Residual S: 2.24% | V) drop-wise to the mixture. | (uncorrected by |
| | (suspension) | QNMR) |
| | Stir for 1 h at 45 ± 5 C. and cool to | The solid is being re- |
| | 25 ± 5 C. (suspension) | slurried with water |
| | Stir for at least 1 h. (suspension) | |
| | Filtrate. | |

To remove DMAc, 3.5 g of Compound 102 (QNMR: 88% ) was re-slurried with 10 V of water, 2.7 g of Compound 102 was obtained with 99.2 A % purity and 77% yield (uncorrected by QNMR).
To increase the yield of crystallization (DMAc/toluene system), 2 V of DMAC and 7 V of toluene was tried, 99.2 A % purity (DMAC are not integrated) of Compound 102 was obtained.

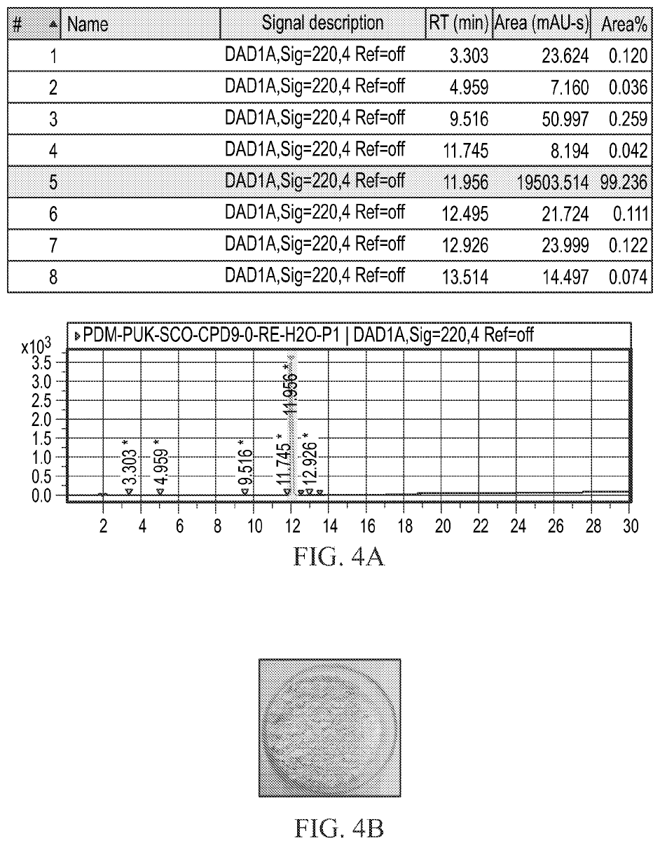

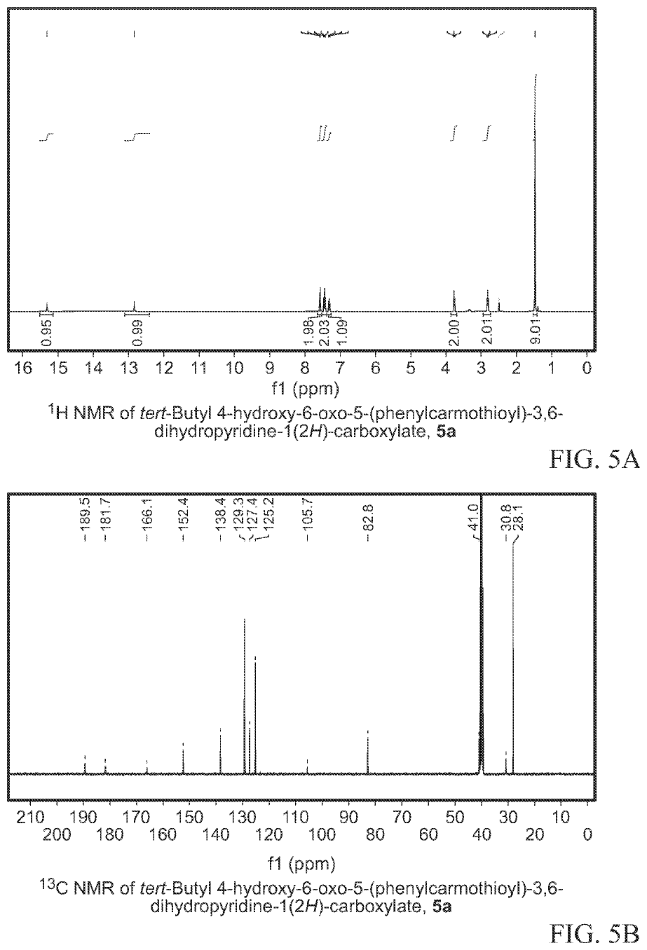

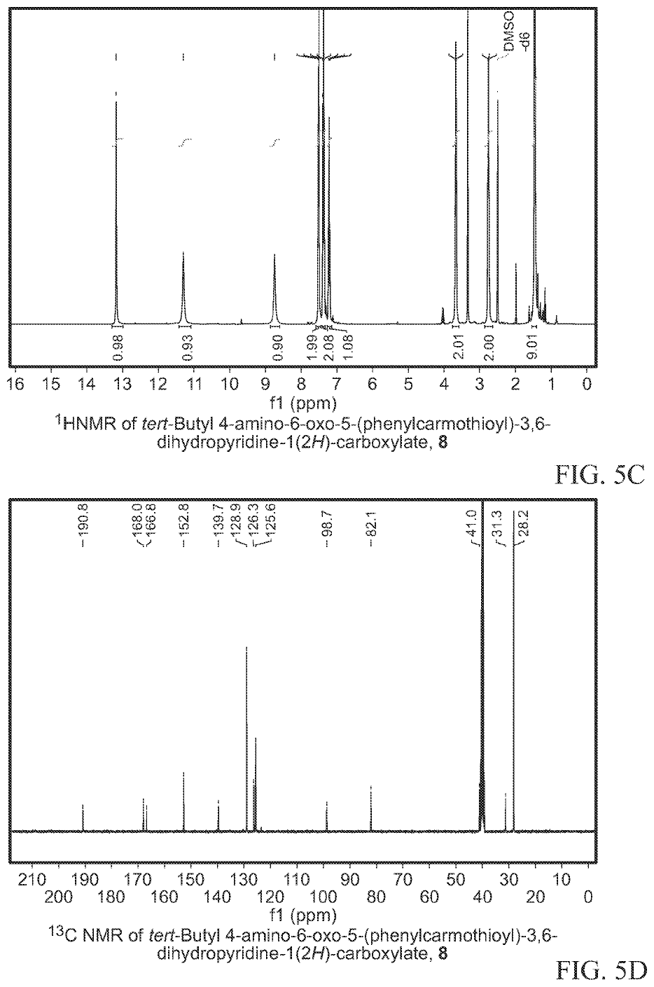

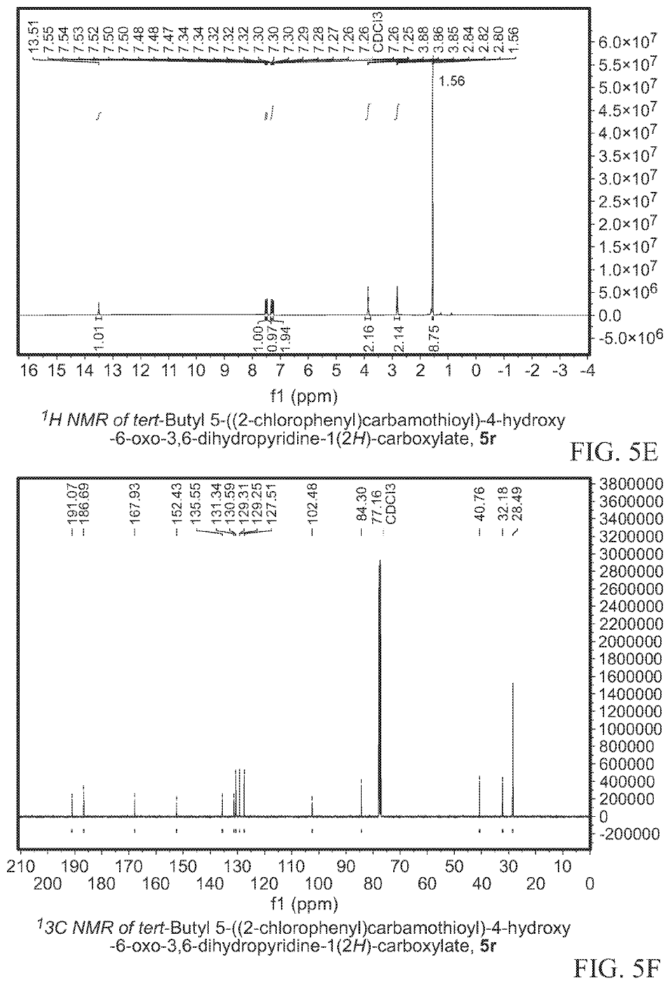

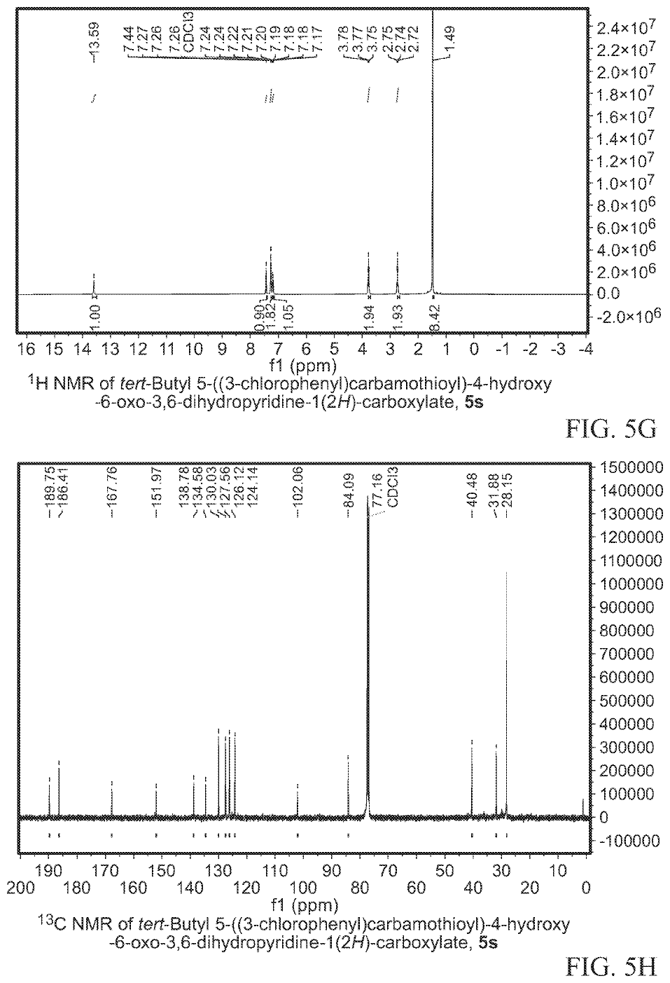

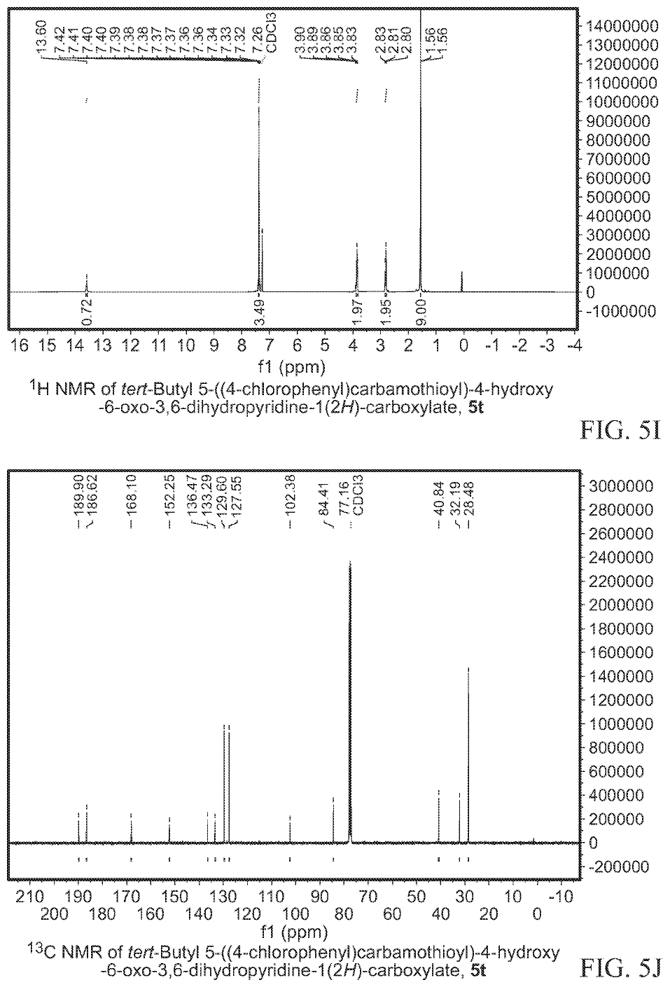

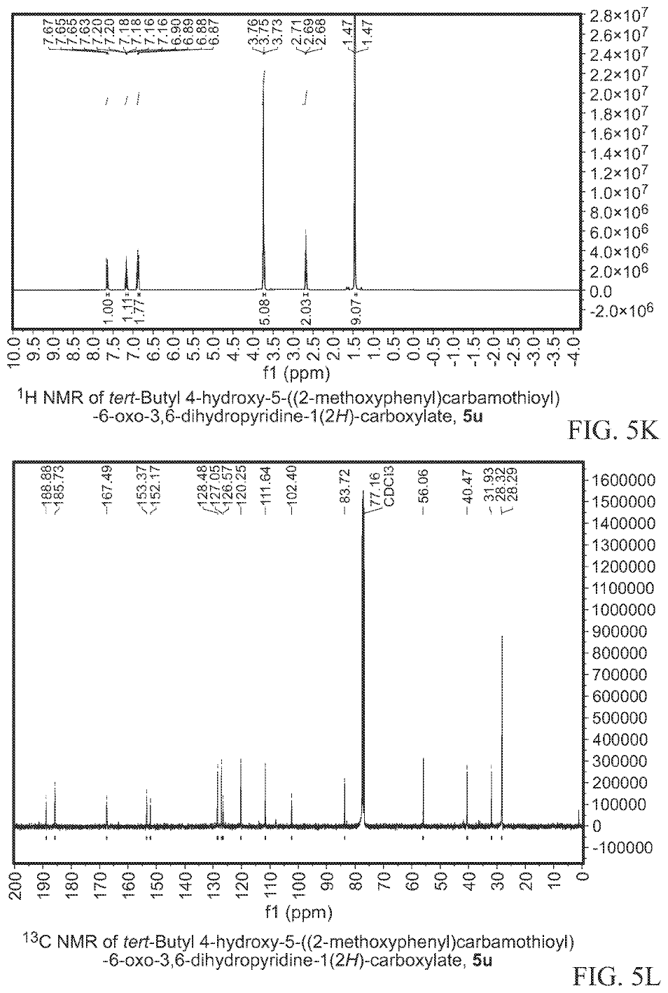

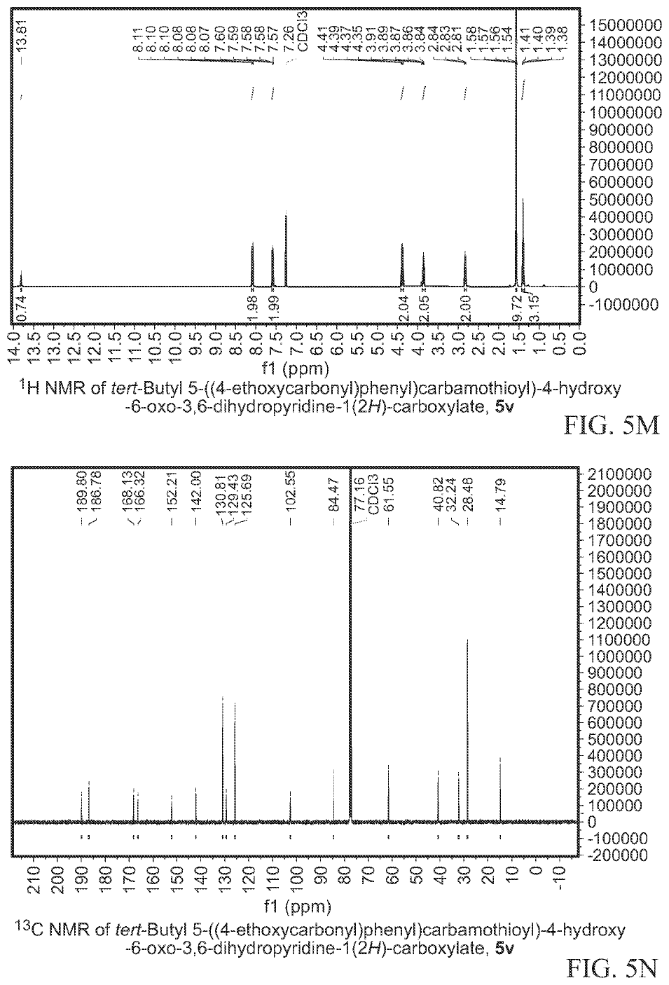

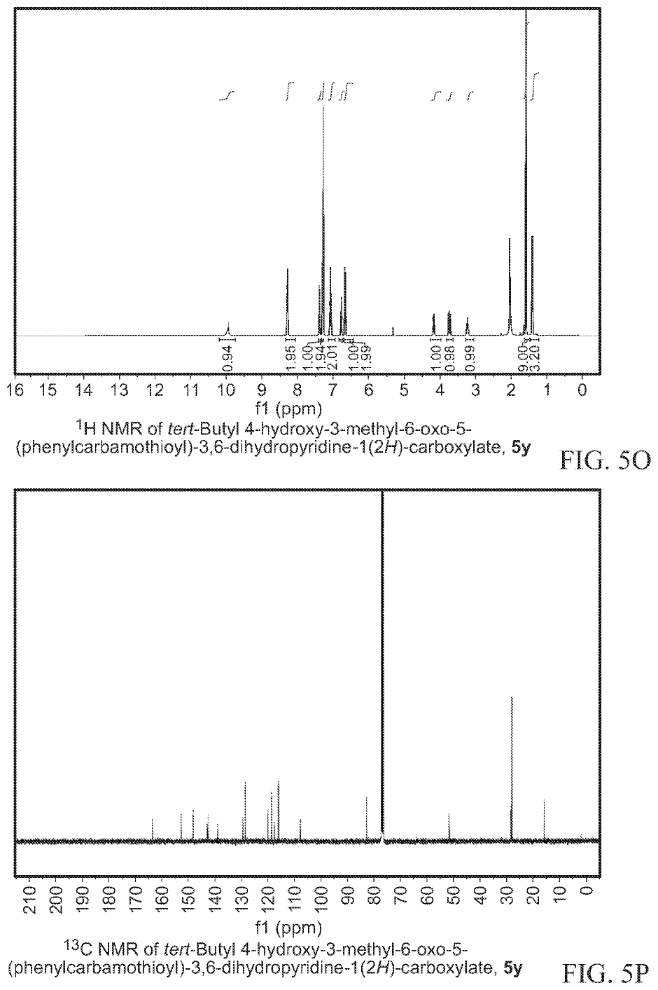

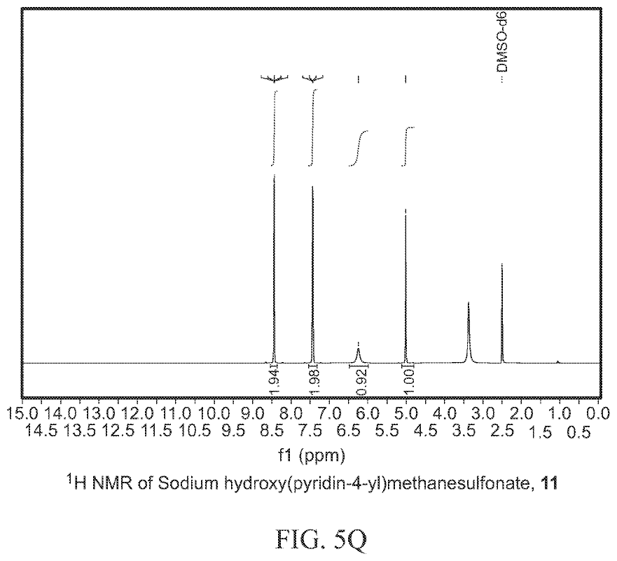

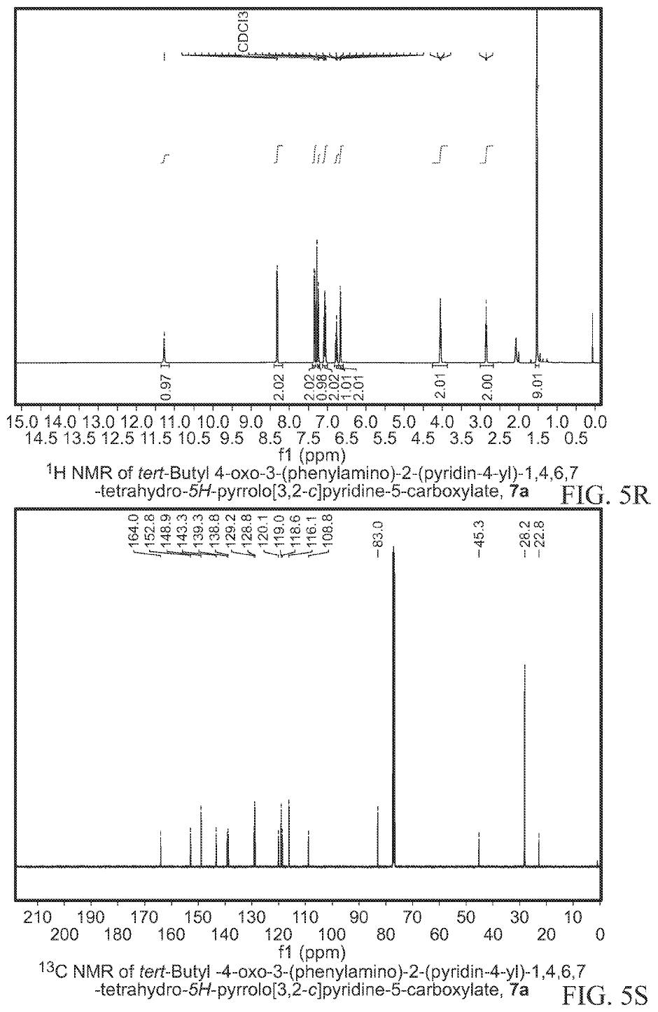

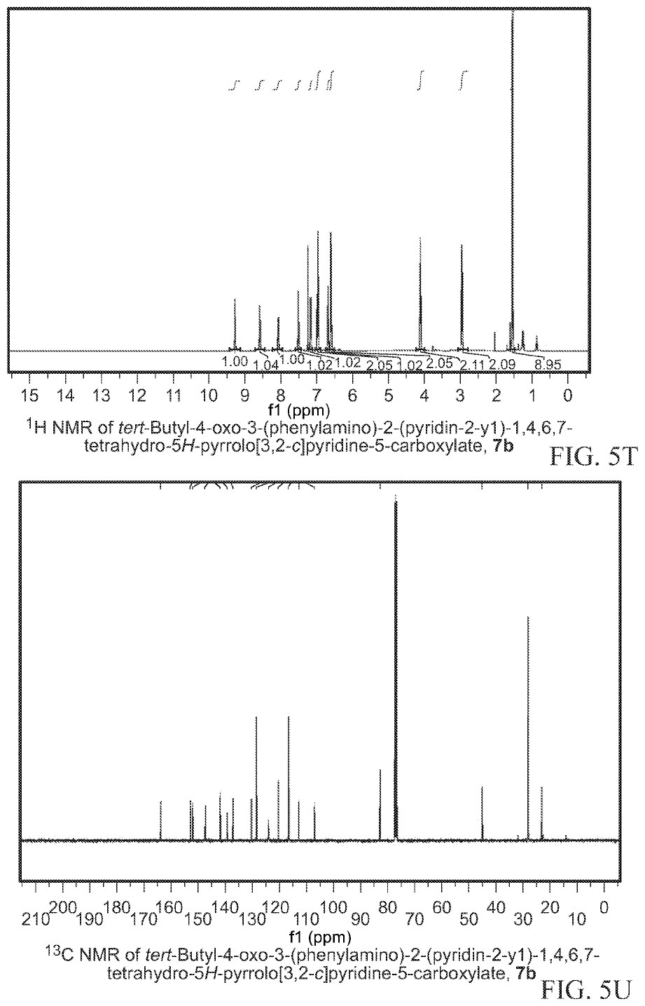

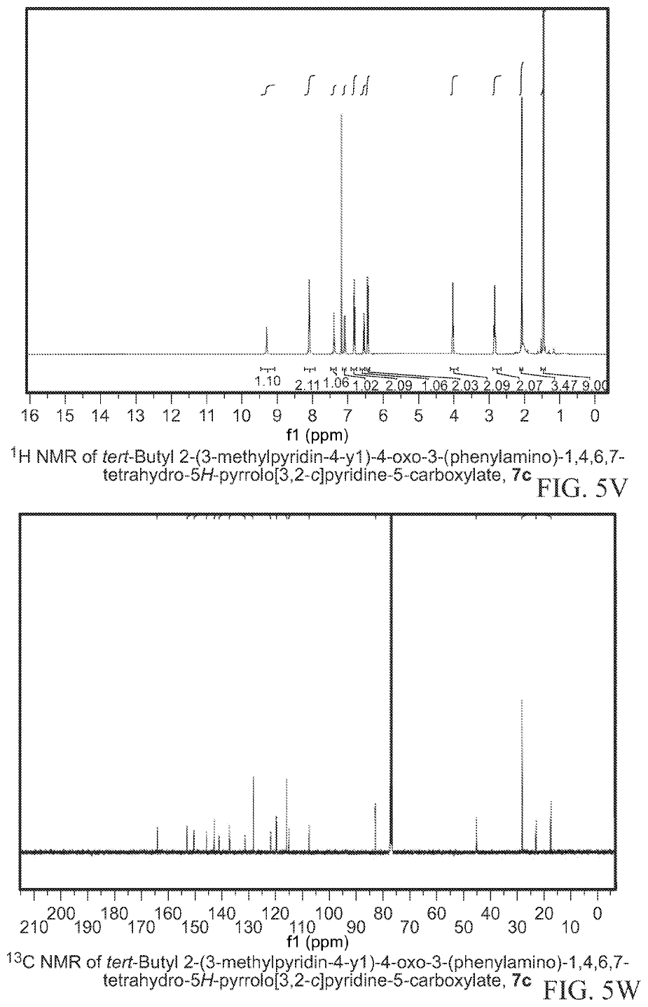

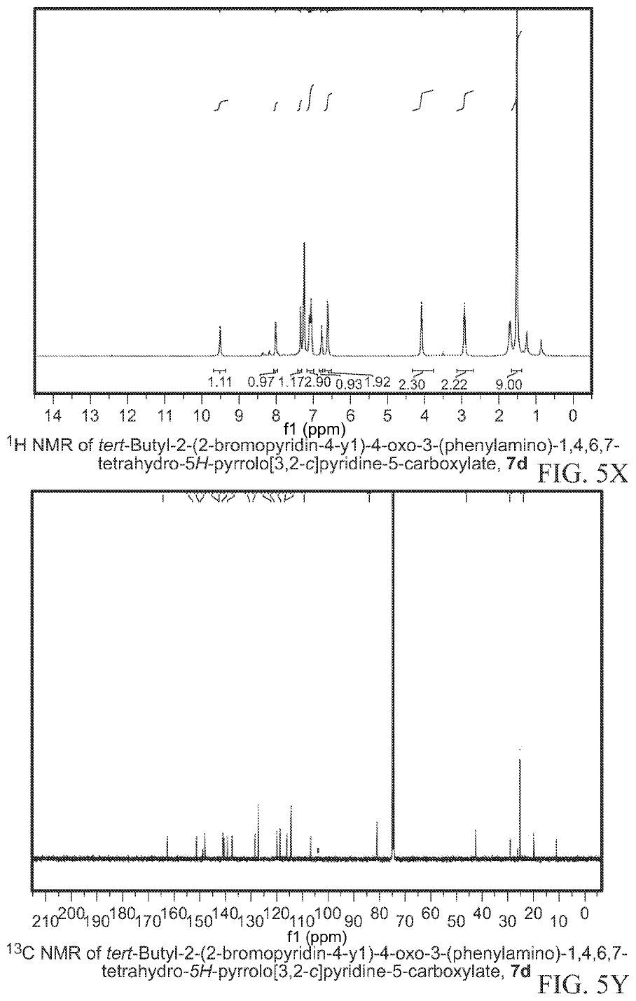

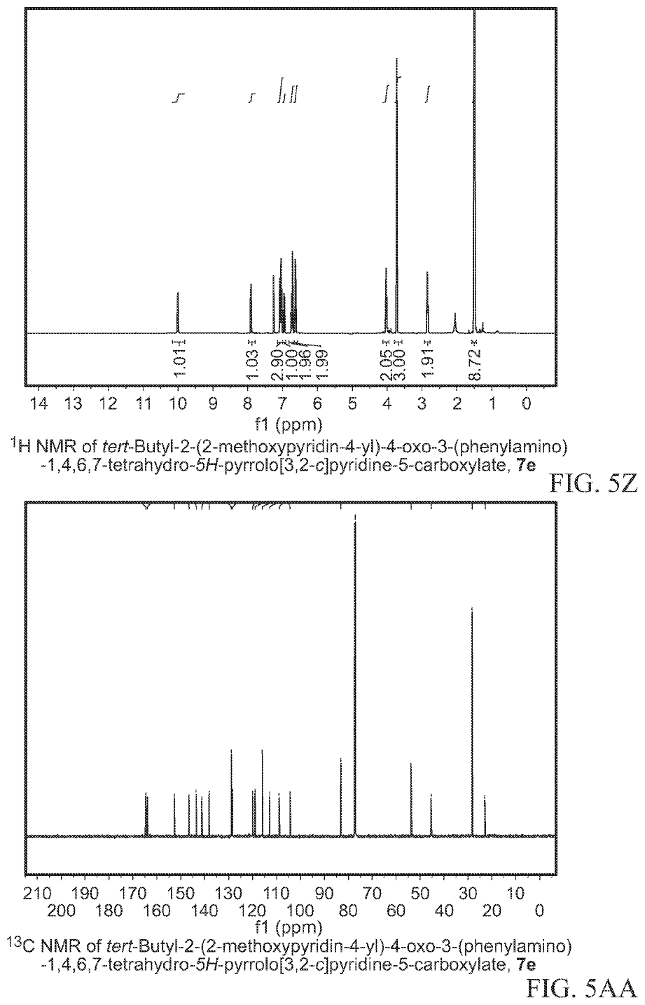

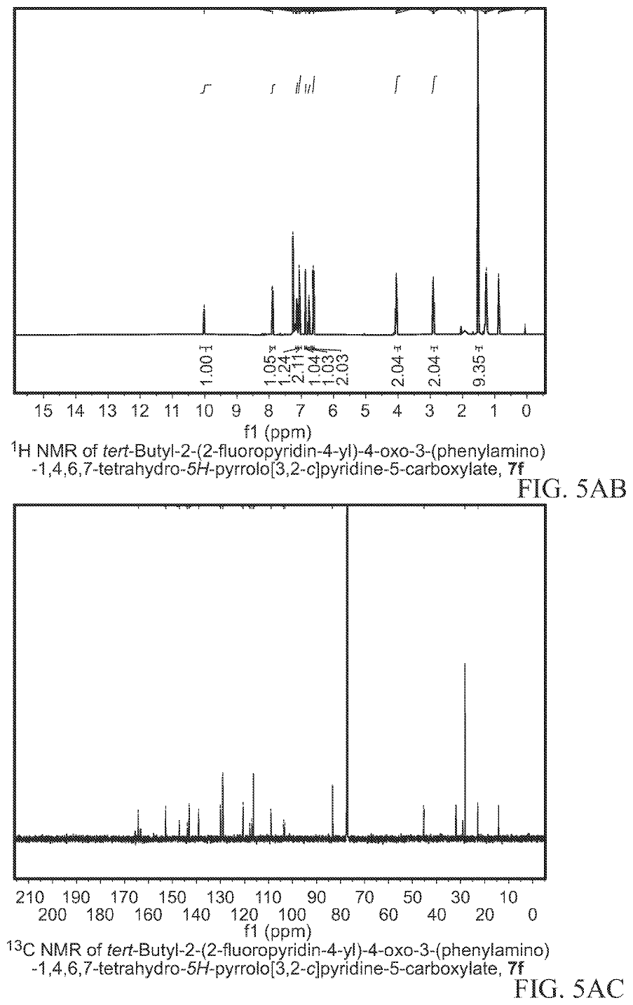

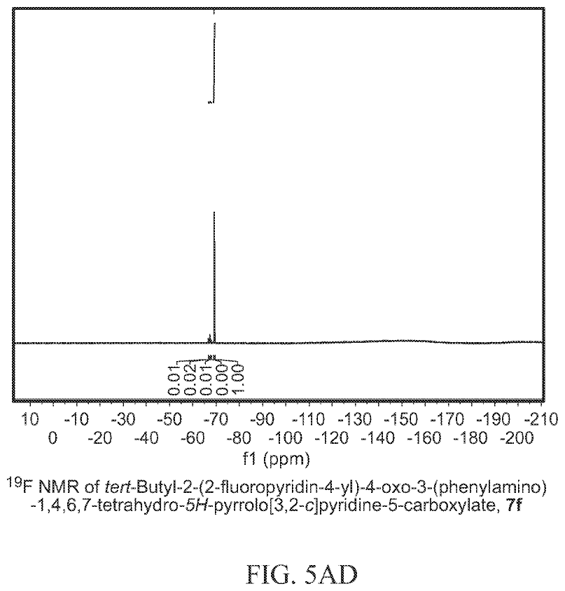

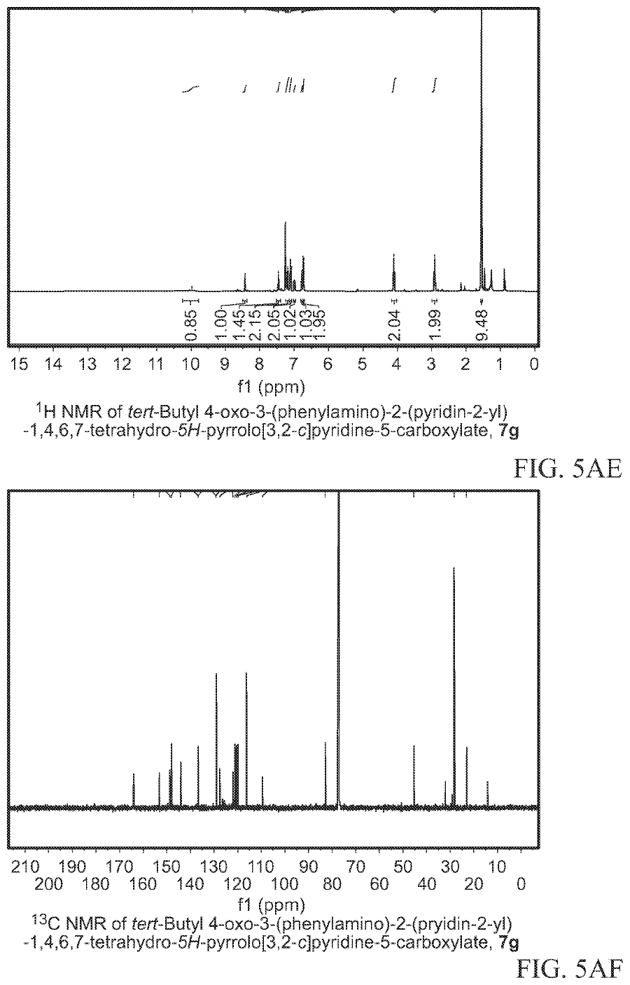

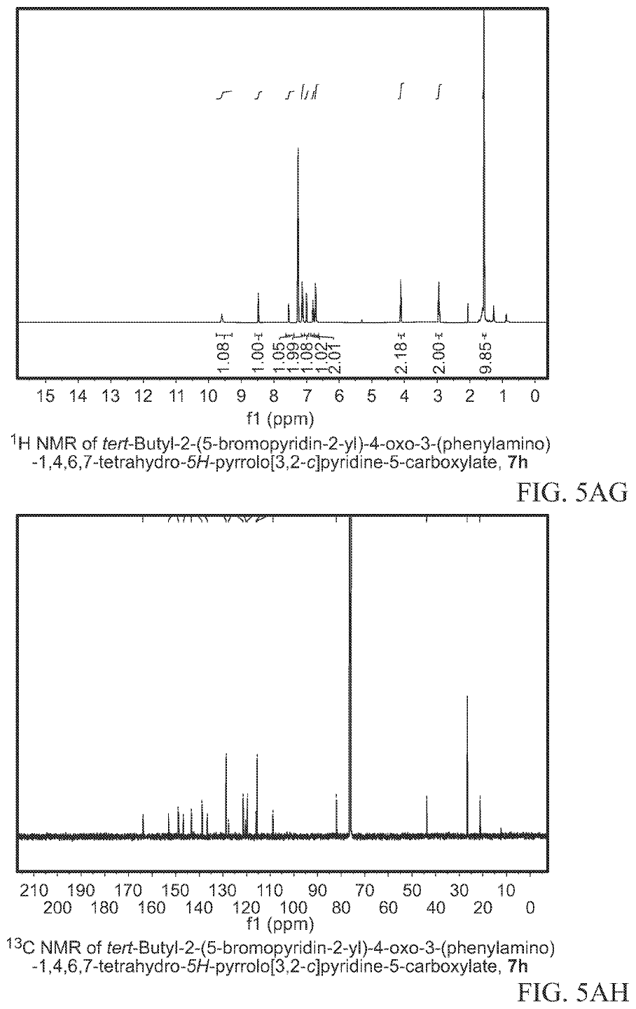

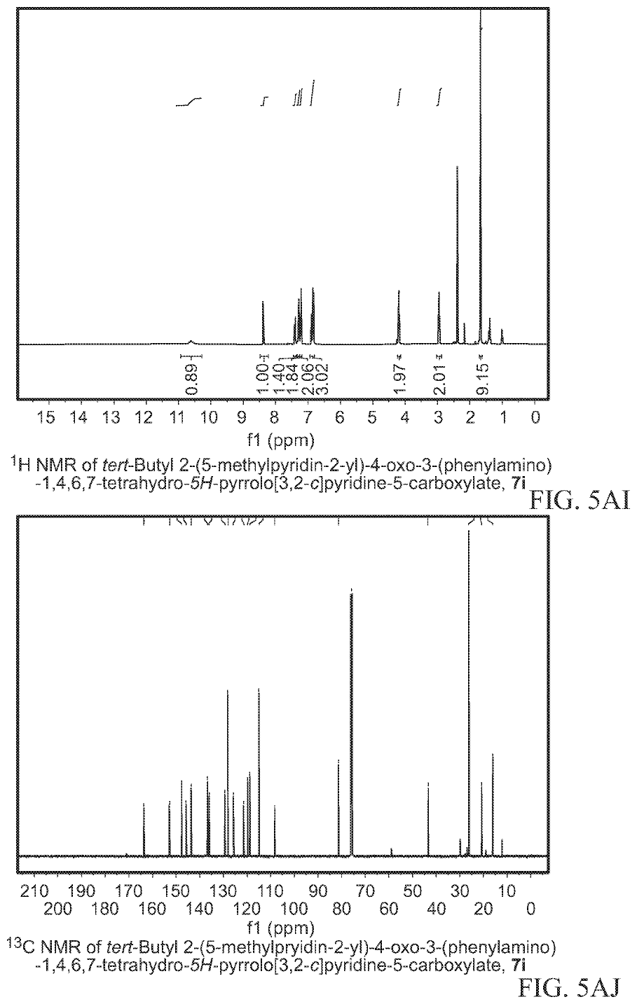

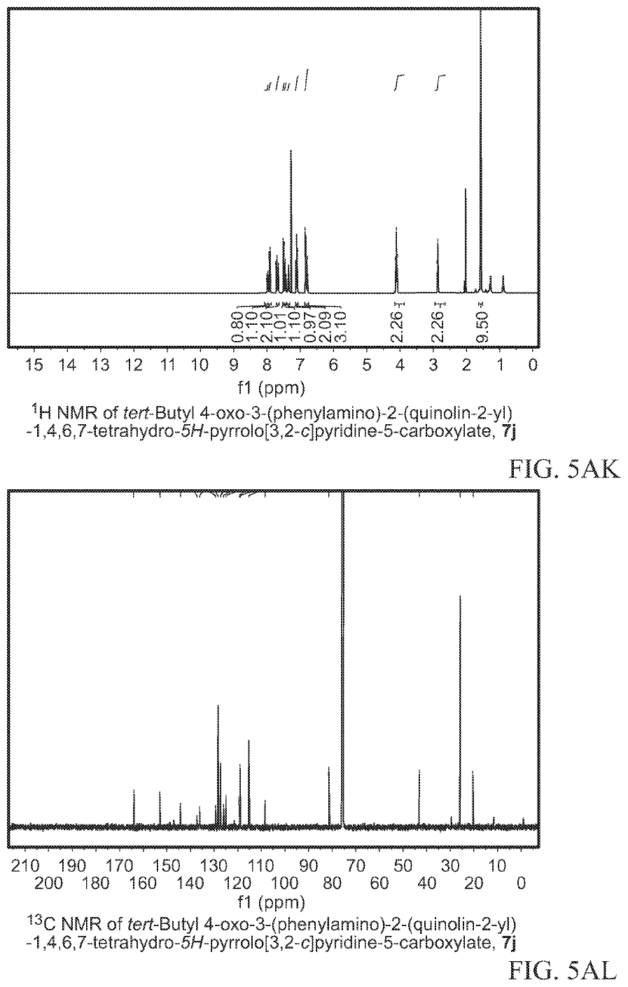

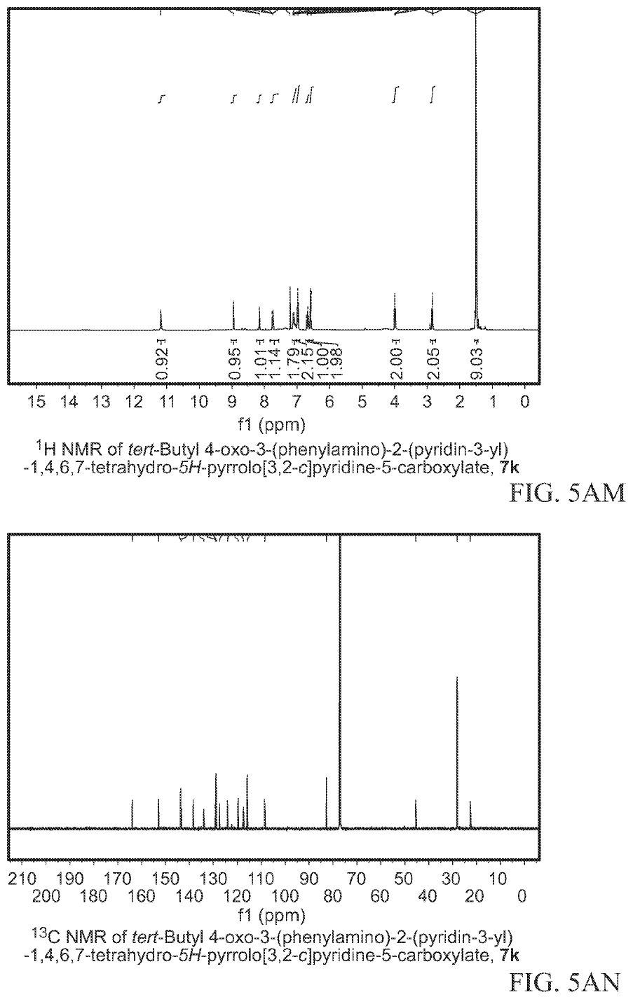

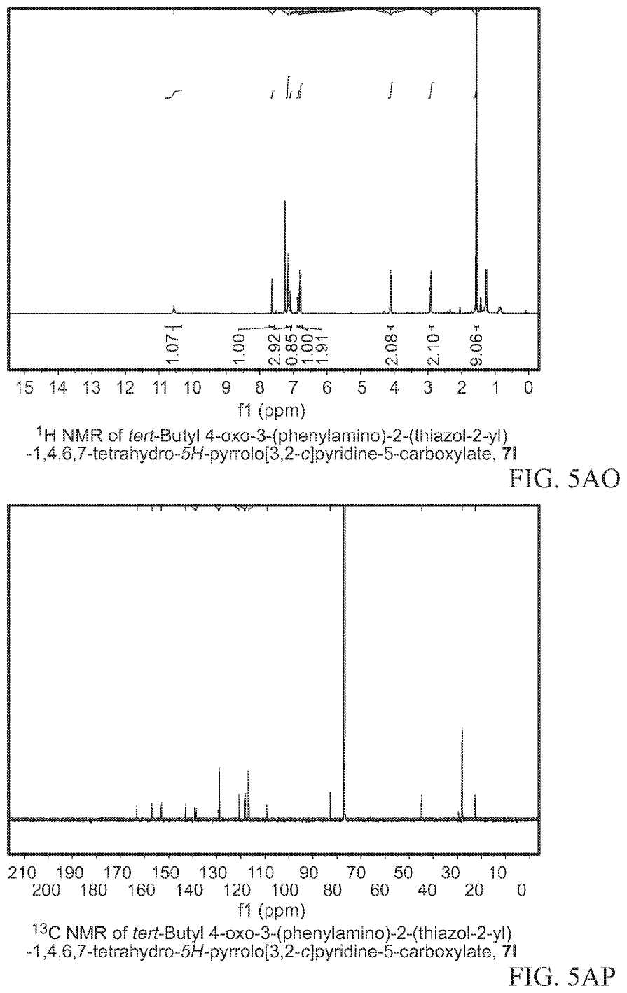

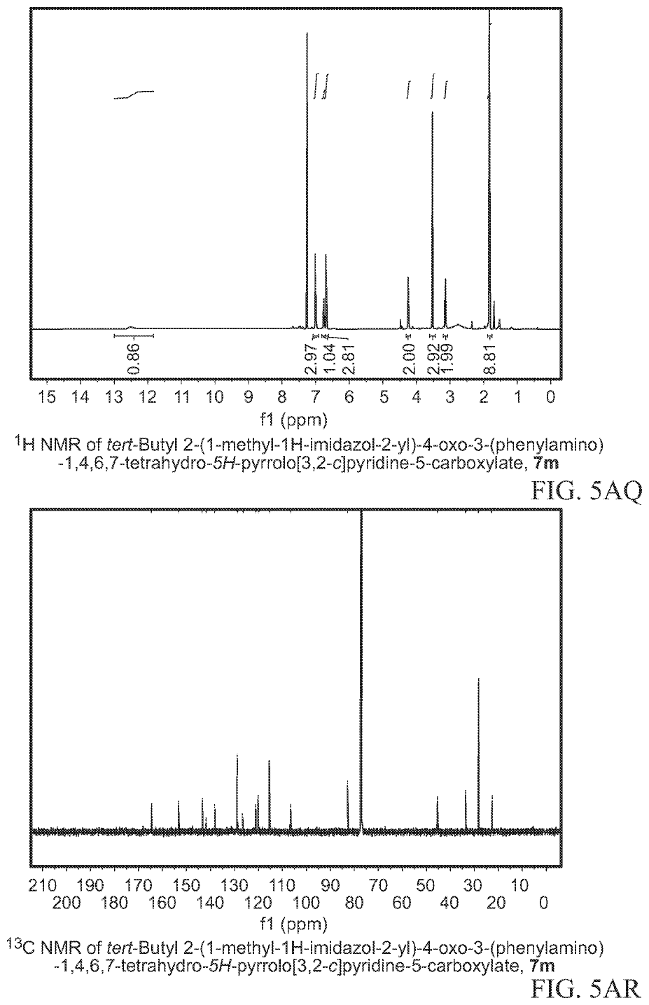

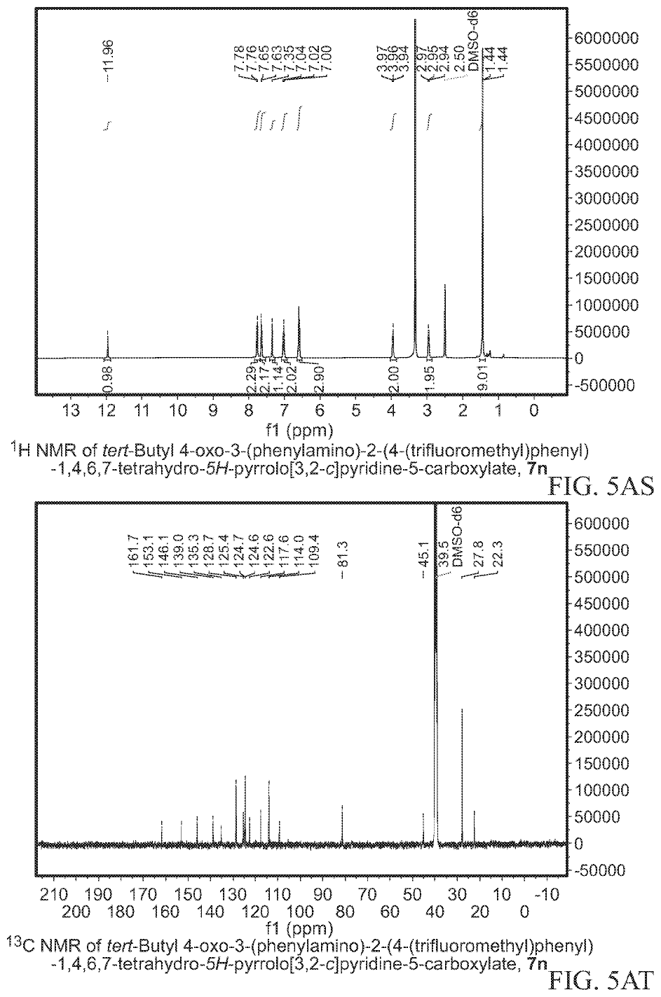

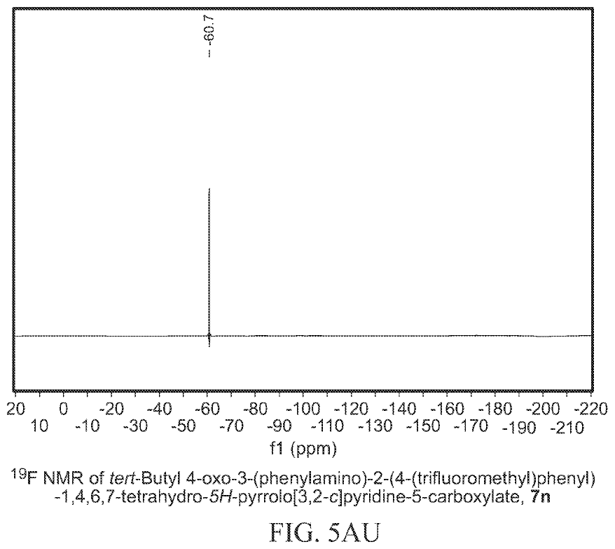

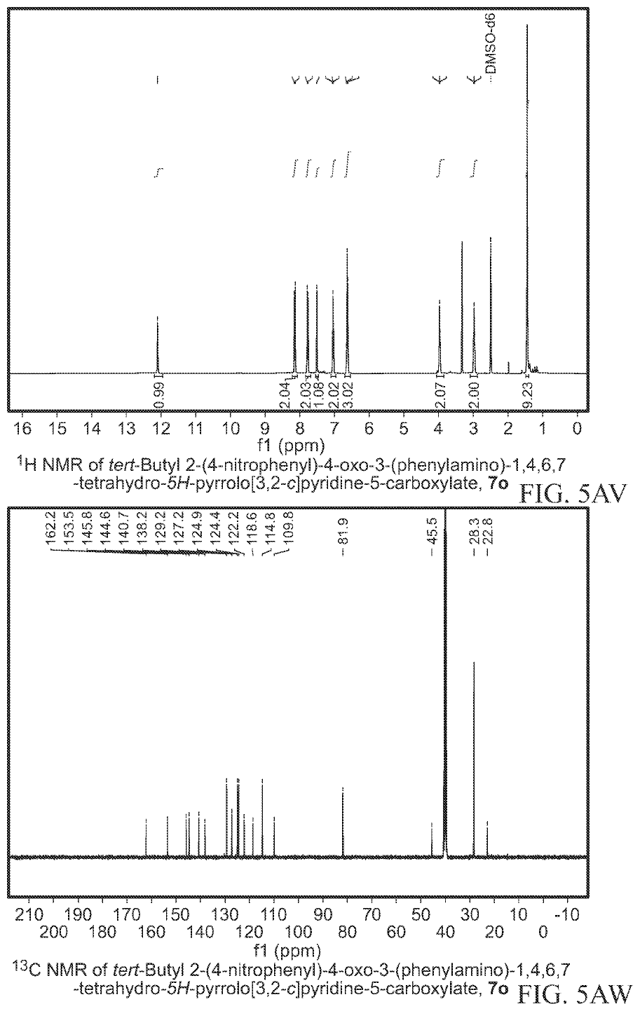

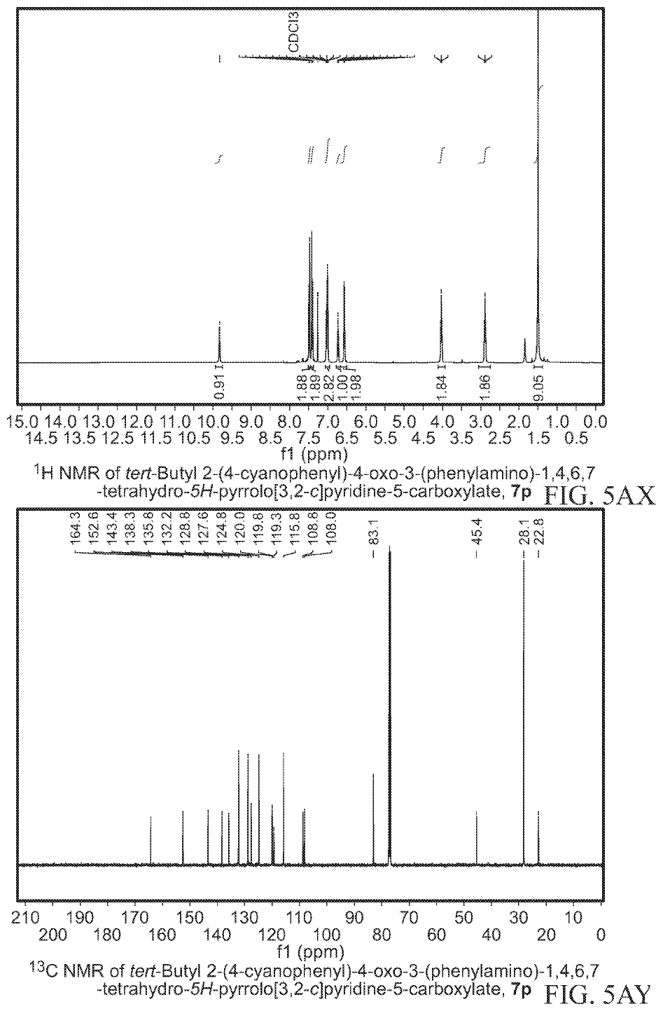

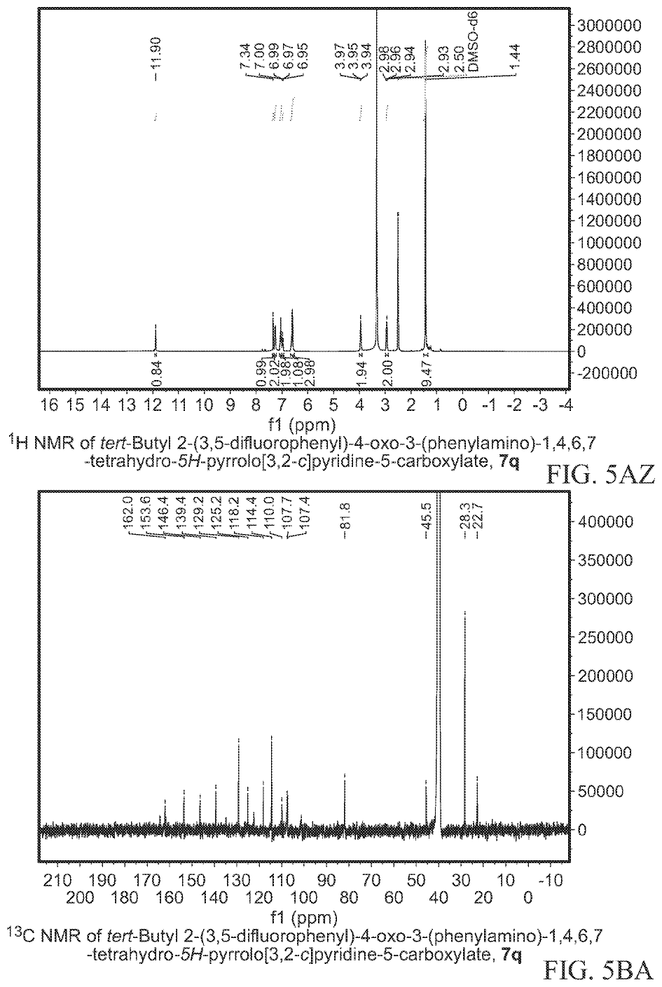

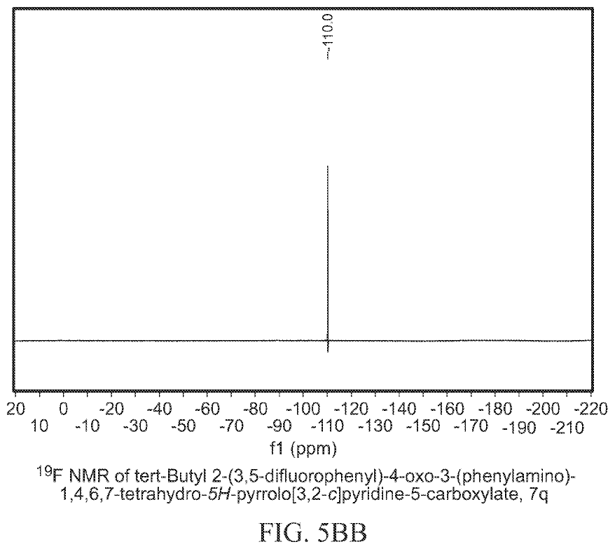

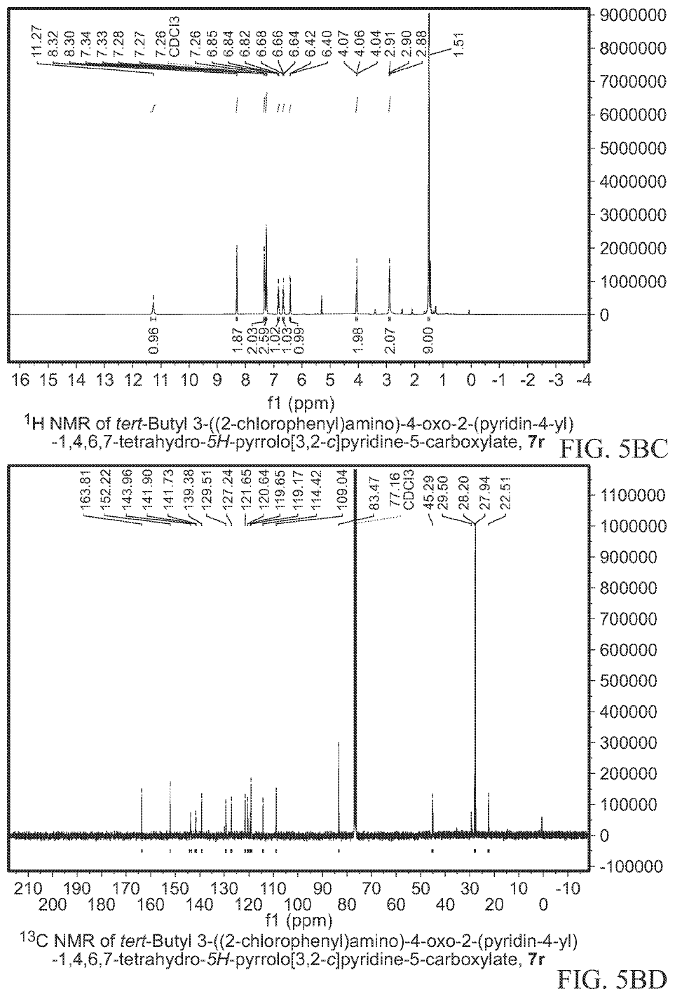

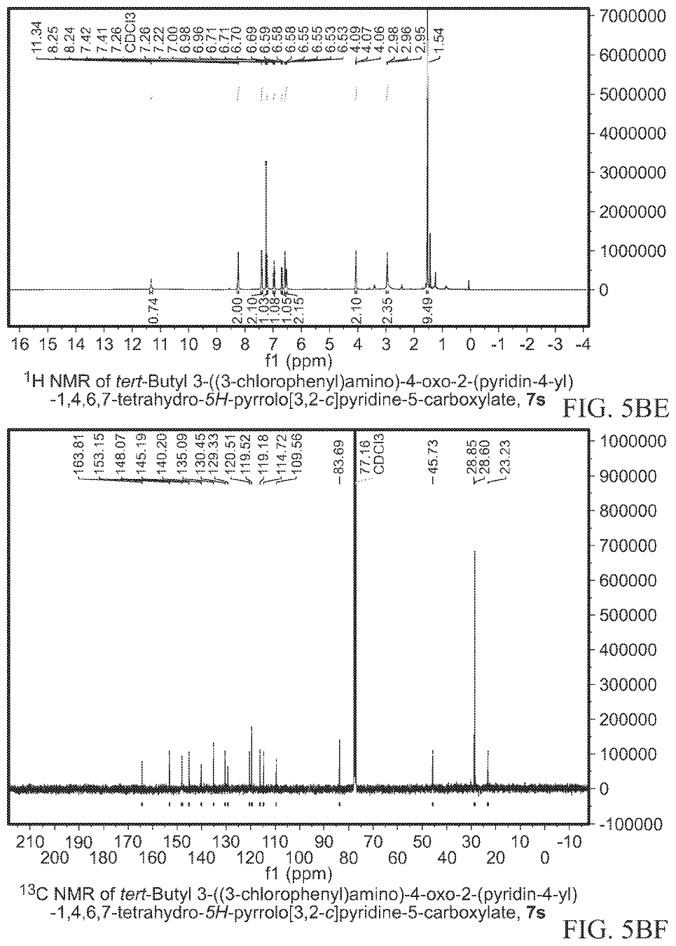

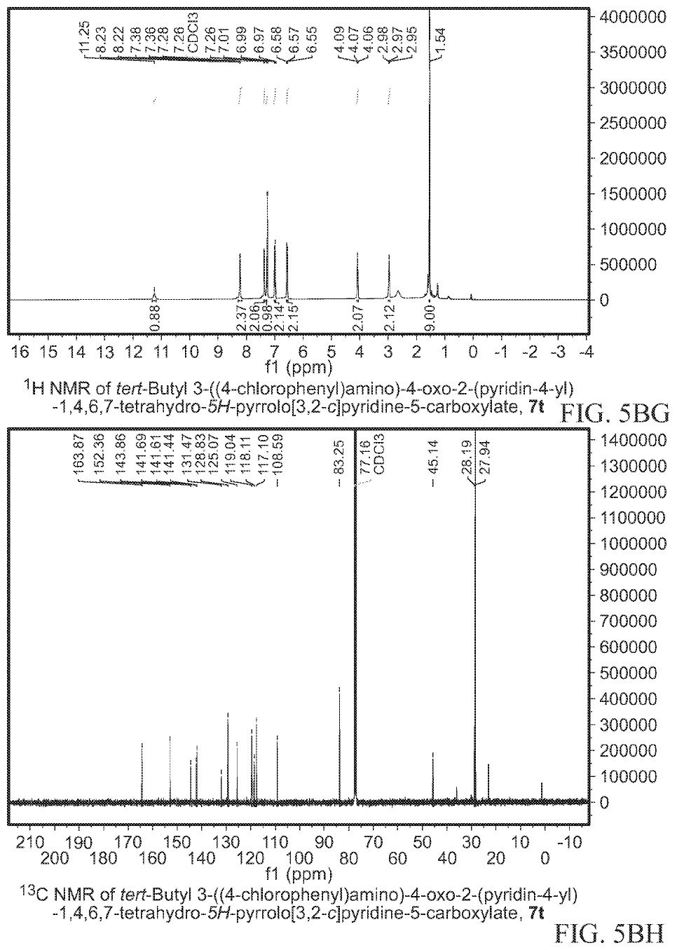

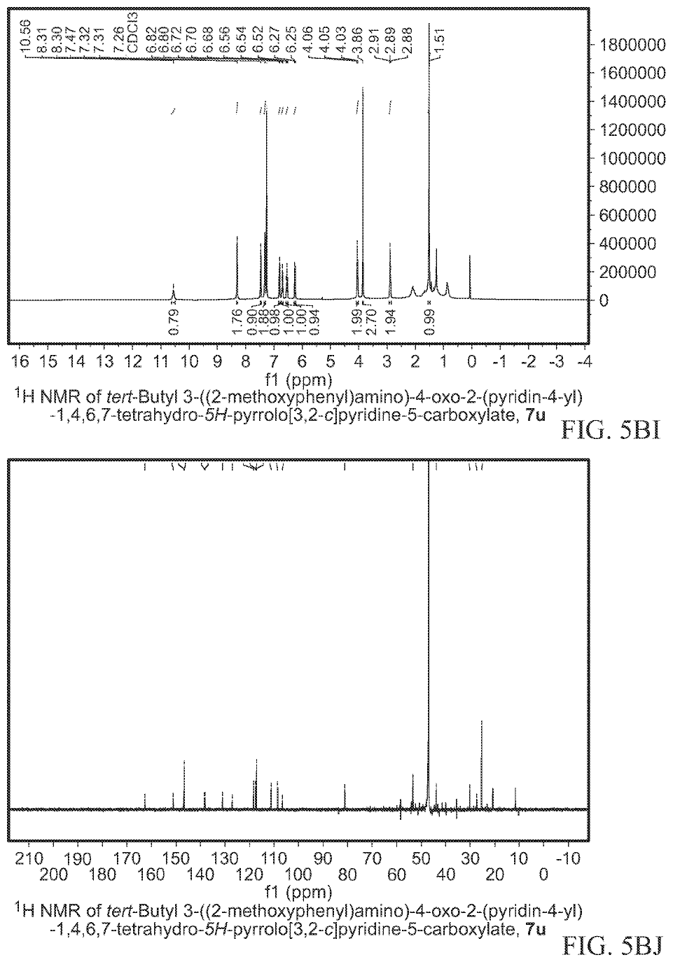

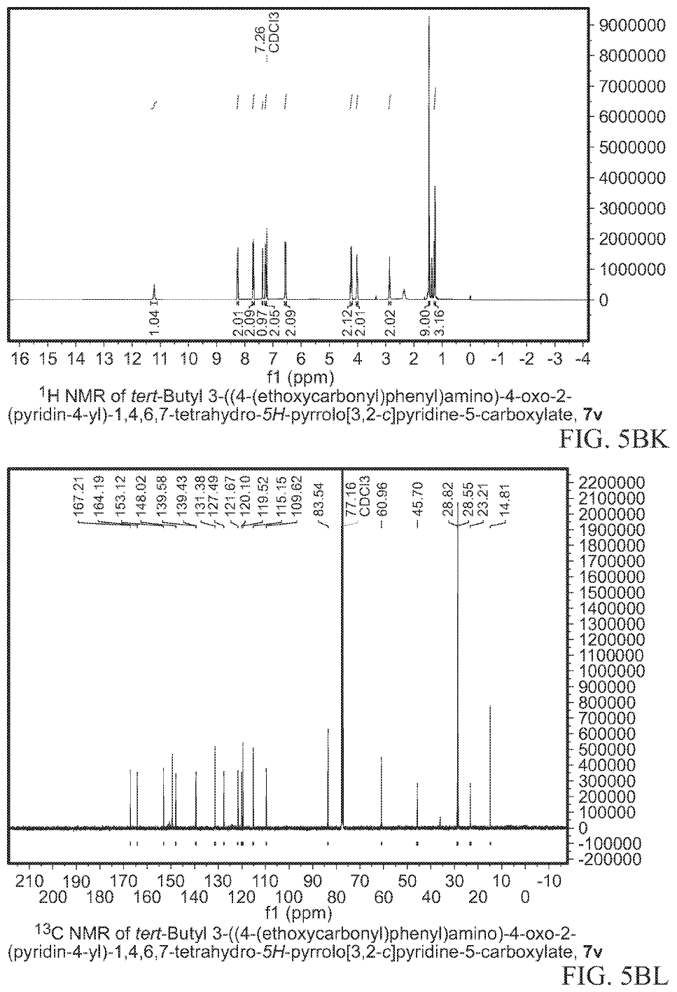

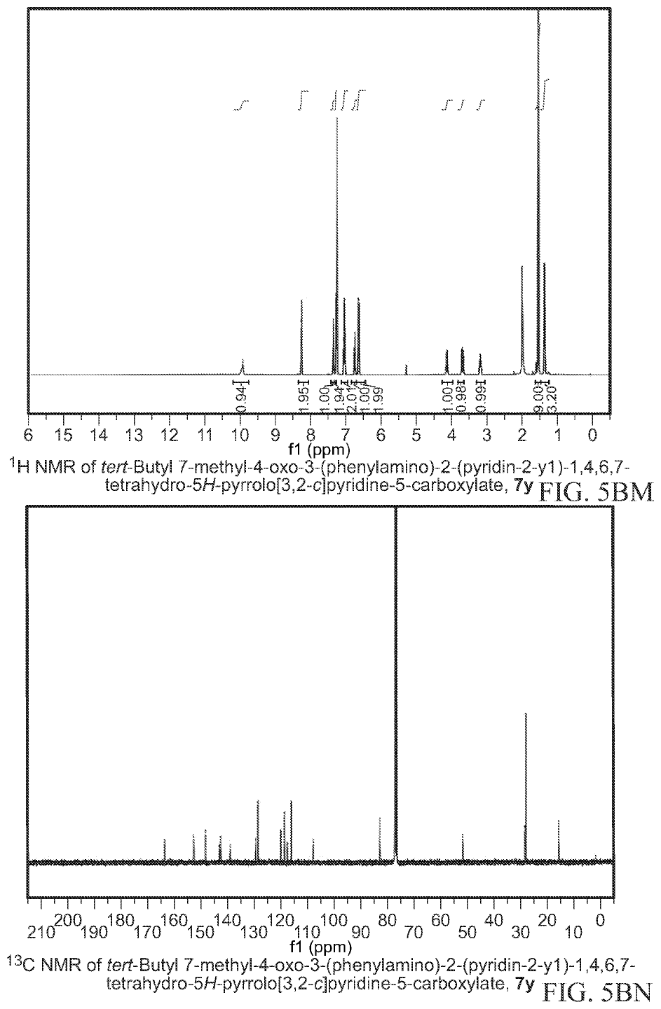

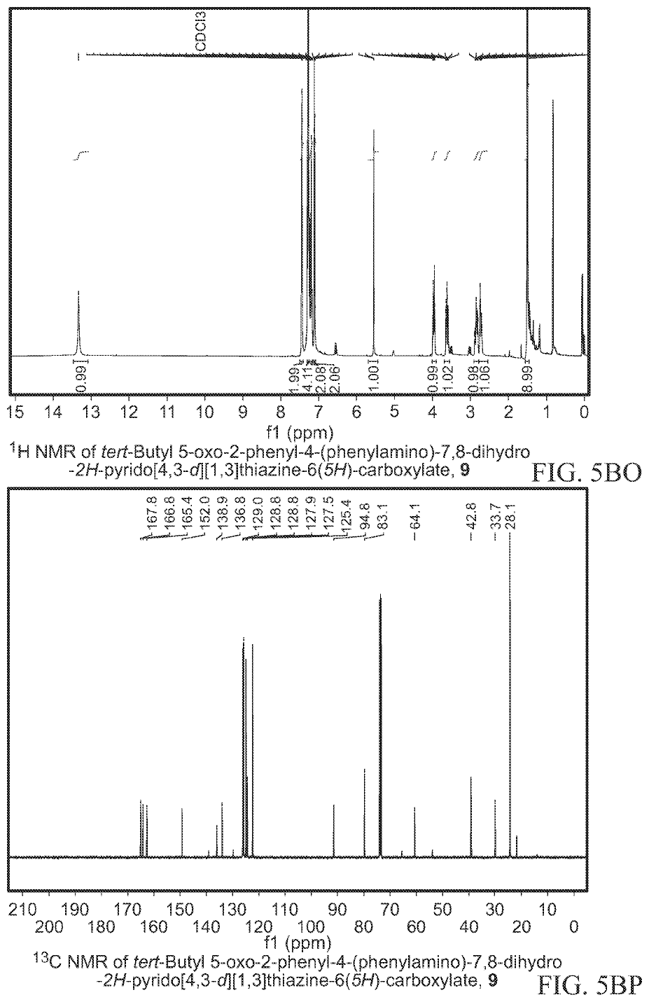

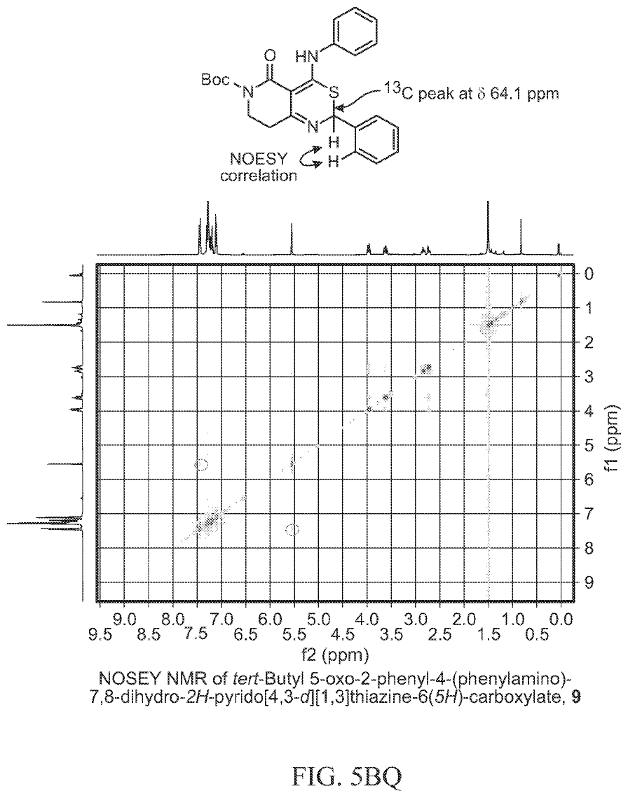

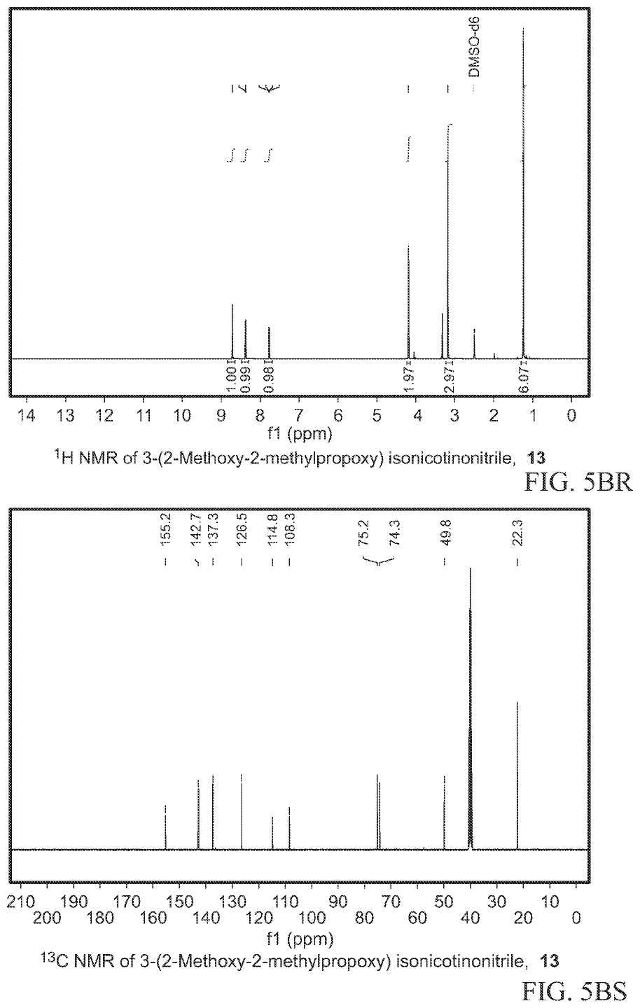

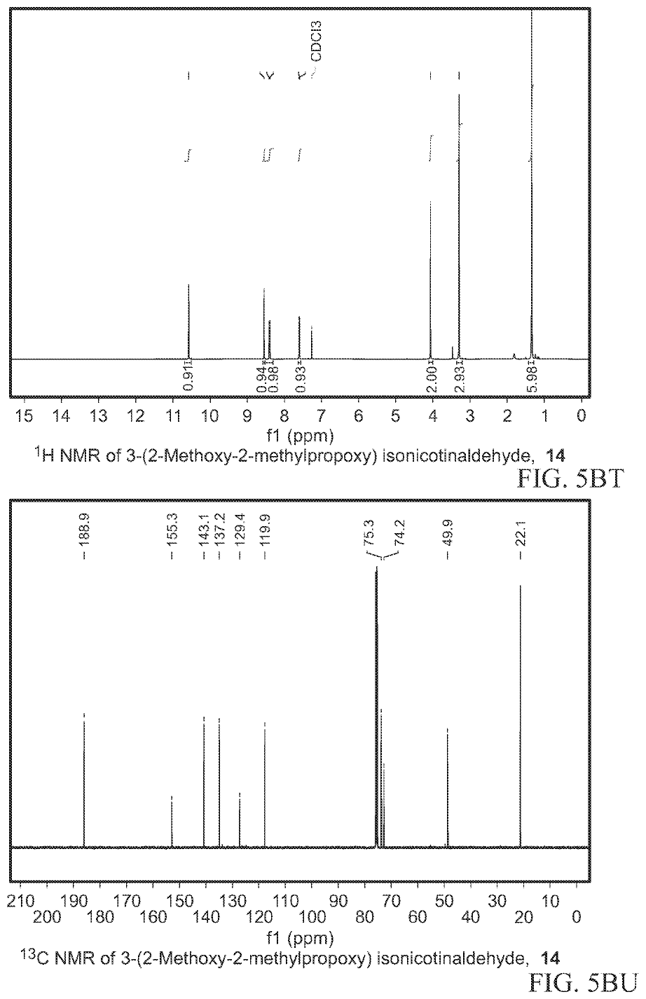

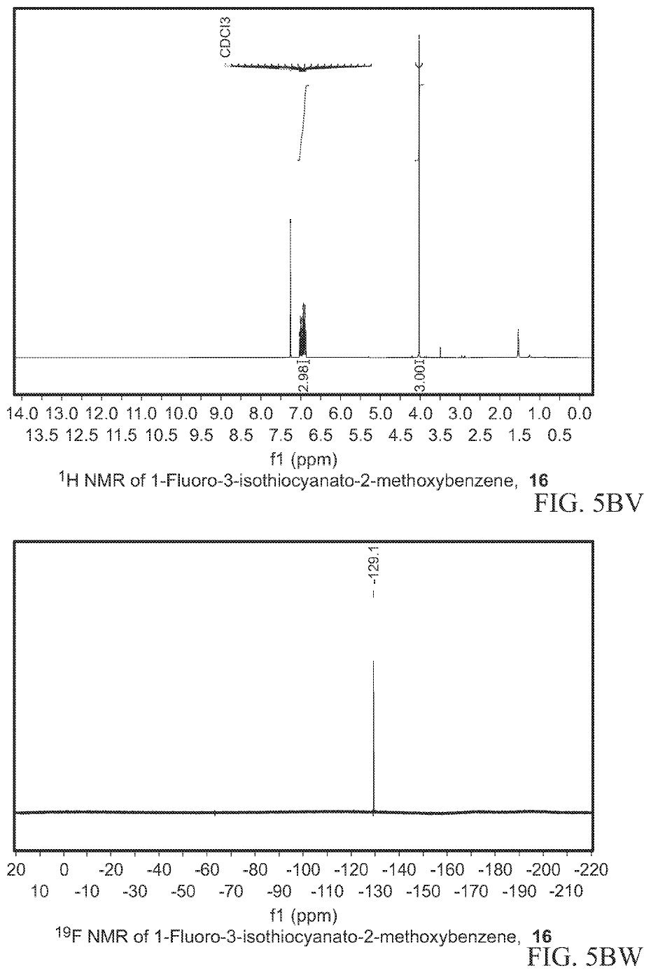

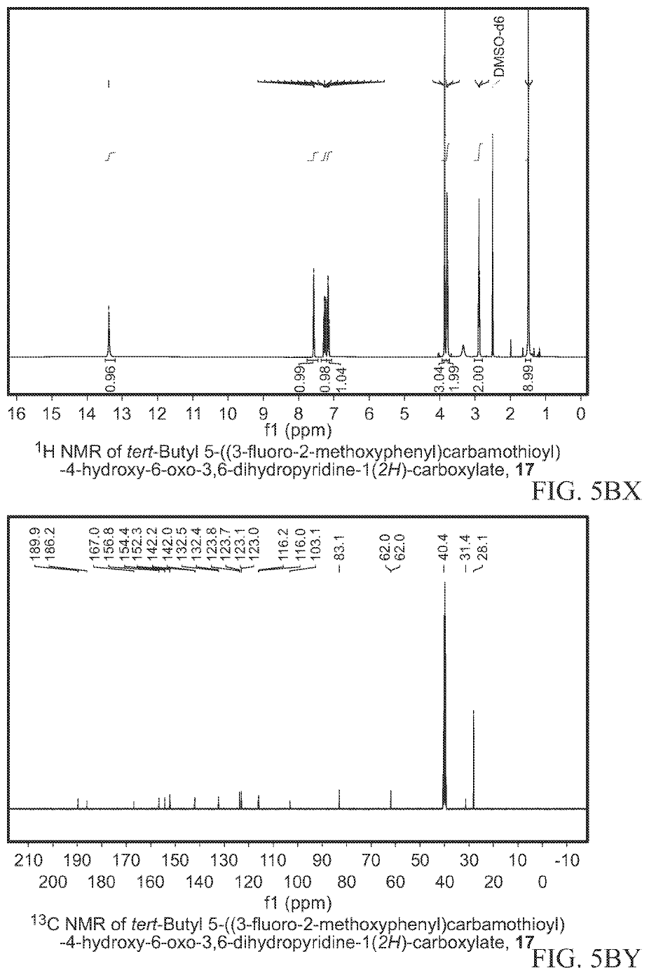

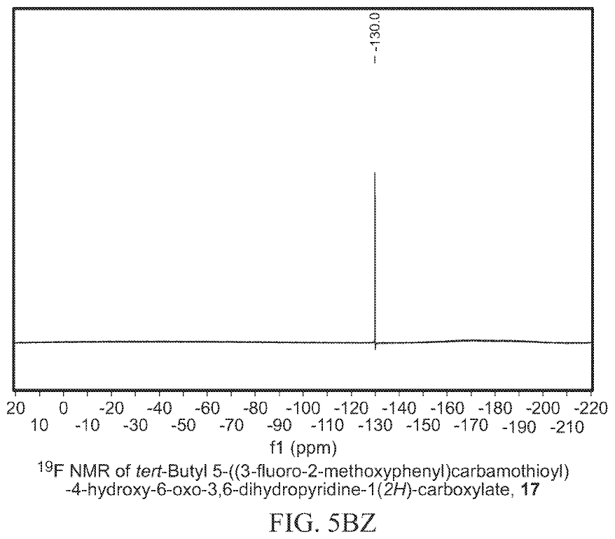

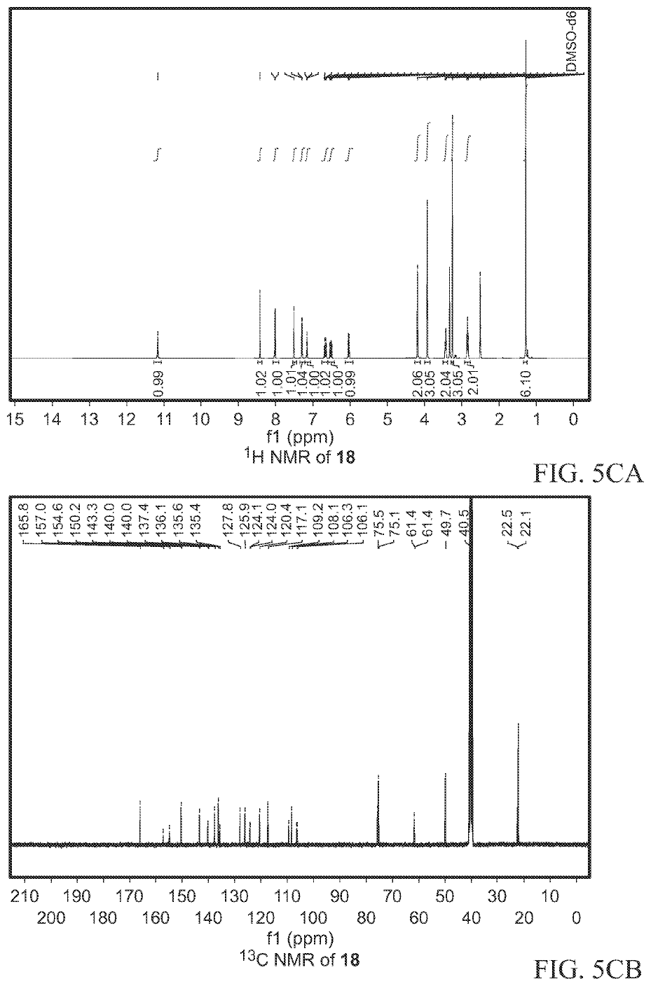

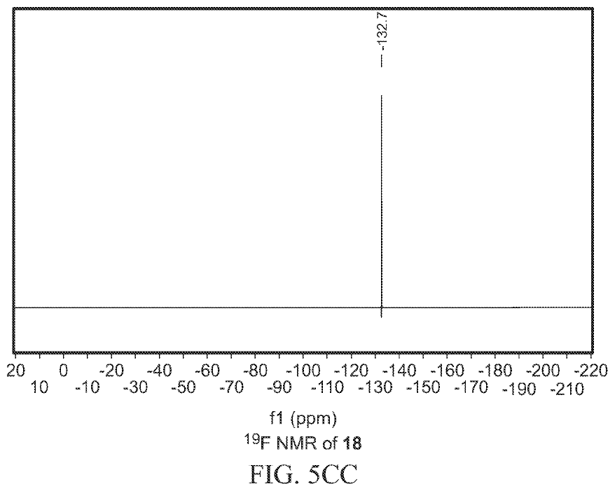

Compound 102
10 g of crude Compound 102 was purified using 2 V of DMAC/7 V of toluene, 8.5 g of Compound 102 was obtained with 99.2 A % purity (DMAC are not integrated) and 85% yield (uncorrected by QNMR). The 8.5 g of Compound 102 is being re-slurried with water to remove DMAc. The LC-MS spectrum and a figure of powder of compound 102 are included in FIGS. 4A-4B.

| Starting Materials | | Conditions | Purification |
|---|---|---|---|
| Step 3 reaction | Relating 5 | Concentrated to 3-4 V, | N/A |
| mixture | g of | added EA (6 V) and TFA | |
| 2.5 A % comp. 2 | comp.2 | (5.0 eq.) | |
| remained | | Reacting for 5 h, 20 A % | |
| 56.4 A % | | Compound 101 was left. | |
| Compound 101, | | After reacting for 16 h, 18 | |
| 5.5 A % | | A % Compound 101 was | |
| Compound 102 | | left. | |
| Assay yield of | Relating 5 | Concentrated to 3-4 V, | N/A |
| Compound 101: | g of | added MTBE (6 V) and | |
| 61.3% | comp.2 | HCl/MeOH (4 M, 3 V) | |
| | | The reaction mixture was | |
| | | very thick. | |
| | Relating | Concentrated to 3-4 V, | Wet cake was used for |
| | 50 g of | added EA (6 V) and | purification directly. |
| | comp.2 | HCl/MeOH (4 M, 3 V) | (recrystallization with |
| | | Reacting for 2 h, 59.5 A % | DMAC/toluene and |
| | | Compound 102, filtered. | slurry with water) |
| | | Added filter cake to | Yellow solid: 25.3 g |
| | | EA/MeOH = 10/1 (10 V), | Purity: 98.0 A % (to be |
| | | added 10% K₂CO₃ (10 V), | re-run) |
| | | filtered. | Yield: 46.8% (from 50 g |
| | | Wet cake: 35 g, Purity: 95.3 | of comp.2) |
| | | A %, Y: 50% (by HPLC | |
| | | assay) | |
| | | (11% of Compound 102 | |
| | | was lost in mother liquors | |
| | | by HPLC assay) | |
| | Relating | Concentrated to 3-4 V, | |
| | 40 g of | added EA (6 V) and | |
| | comp.2 | HCI/MeOH (4 M, 3 V) | |
| | | Reacting for 2 h, 58.3 A % | |
| | | Compound 102 | |
| | | Concentrated to 5-6 V, | |
| | | switched with EA (6 V*2), | |
| | | added EA/MeOH = 4/1 (5V) | |
| | | Added 10% K₂CO₃ (10 V), | |
| | | filtered. | |
| | | 23 g of Compound 102, | |
| | | Purity: 96.1% Y: 47% (by | |
| | | HPLC assay) | |
| | | (13% of Compound 102 | |
| | | was lost in mother liquors | |
| | | by HPLC assay) | |

| Compound 102 | Conditions | Result |
|---|---|---|
| 10 g crude | Stage 1: | 8.5 g |
| P: 95.2 A % | DMAC (2 V) and toluene (2 V) at | P: 99.0 A % |
| QNMR: 88% | 75 ± 5 C. for 1 h. (clear solution) | (DMAC is not |
| Residual S: 2.24% | Cool to 45 ± 5 C. and charge toluene (5 | integrated) |
| | V) drop-wise to the mixture. | Yield: 85% |
| | (suspension) | |
| | Stir for 1 h at 45 ± 5 C. and cool to 25 ± 5 | (uncorrected by |
| | C. (suspension) | QNMR) |
| | Stir for at least 1 h. (suspension) | |
| | Filtrate and wash. | |
| | Stage 2: | 6.5 g |
| | 8.5 g of Compound 102 was slurried | P: 99.7 A % |
| | with water (10 V) at 45 ± 5° C. for 1 h. | Yield: 65% (from 10 |
| | (suspension) | g of crude Compound |
| | Cool to 5 ± 5 C. and stir for 1 h. | 102) |
| | Filtrate and wash. | QNMR: 99.5% |
| | | Residual S: 119 ppm |

10 g of Compound 102 (assay: 88% ) was purified via stage 1 (crystallization by DMAc/toluene) and stage 2 (slurry with water):
Stage 1, 8.5 g (containing ˜˜12% w/w of DMAc by QNMR) of Compound 102 was obtained with 99.0 A % purity (DMAc is not integrated) and 8500 yield (uncorrected by QNMR).
Stage 2, 6.5 g of Compound 102 was obtained with 99.7 A % purity and 99.5% potency by QNMR. The yield was 65% yield (for 10 g of crude Compound 102) and the residual S was 119 ppm.

| | Compound | | |
|---|---|---|---|
| Lot. | 102 | Conditions | Result |
| 5 | 5 g | DMAC (1 V) and toluene (3 V) at | Wet cake |
| | | 75 ± 5° C. for 1 h. (suspension) | P: 99.6 A % |
| | P: 95.2 A % | Cool to 45 ± 5 C. and charge toluene (5 | (DMAC is not |
| | | V) drop-wise to the mixture. | |
| | | (suspension) | |
| | | Stir for 1 h at 45 ± 5 C. and cool to 25 ± 5 | |
| | | C. (suspension) | |
| | QNMR: 88% | Stir for at least 1 h. (suspension) | integrated) |
| | Residual S: | Filtrate and wash. | |
| | 2.24% | The wet cake form stage 1 was slurried | 3.5 g |
| | | with water (10 V) at 45 ± 5° C. for 1 h. | P: 99.6 A % |
| | | (suspension) | Yield: 70% (from 5 g of |
| | | Cool to 5 ± 5 C. and stir for 1 h. | crude Compound 102) |
| | | Filtrate and wash. | QNMR: 99.5% |
| | | | Residual S: 119 ppm |
| 6 | 5 g | DMAC (0.5 V) and toluene (3.5 V) at | 3.6 g |
| | P: 95.2 A % | 75 ± 5 C. for 1 h. (suspension and thick) | P: 96.9 A % |
| | QNMR: 88% | Cool to 45 ± 5 C. and charge toluene (5 | Yield: 72% (from 10 g |
| | Residual S: | V) drop-wise to the mixture. | of crude Compound |
| | 2.24% | (suspension) | 102) |
| | | Stir for 1 h at 45 ± 5 C. and cool to 25 ± 5 | Residual S: 423 ppm |
| | | C. (suspension) | QNMR: 98.5% |
| | | Stir for at least 1 h. (suspension) | |
| | | Filtrate and wash. | |
| | | The wet cake form stage1 was slurried | |
| | | with water (10 V) at 45 ± 5° C. for 1 h. | |
| | | (suspension) | |
| | | Cool to 5 ± 5 C. and stir for 1 h. | |
| | | Filtrate and wash. | |

In order to reduce the loss during purification, different ratio of DMAC/toluene (1 V/3 V, 0.5 V/3.5 V) were tried on 5 g scale:
DMAC/toluene=1 V/3 V, 3.5 g of Compound 102 was obtained with 99.6 A % purity and 99.5% potency by QNMR, the recovery yield was 70% and the residual S was 119 ppm.
DMAC/toluene=0.5 V/3.5 V, 3.6 g of Compound 102 was obtained with 96.9 A % purity and 98.5% potency by QNMR, the recovery yield was 72% and the residual S was 423 ppm.
The mass balance data of step 3&4 was collected, based on the mass balance data, around 2000 of product was lost during work-up and purification.

| | | | | Step 4: | | |
|---|---|---|---|---|---|---|
| | Step 3: | | | Starting | | |
| | Starting | Reaction | | Materials | | |
| | Materials | Conditions | | Reaction | | |
| Lot | Solution 1 | Temp./Time | IPC | Conditions | IPC | Result |
| 8 | Solution 12: | Solution 2 | 2: 22.4 min: | Concentrated | Compound | 24 g |
| | 50 g (1.0 | added to | 2.3A % | to 3-4 V | 101: 17.9 | Purity: 99.6A |
| | eq.) | solution 1 | Compound | charged EA | min: 0.2 | % |
| | NH₄OAc: 5.0 | at 95 ± 5 C. | 101: 17.9 min: | (6V) | A % | Yield: 43% |
| | eq. | for 6 h | 55.0 A % | Charged4M | | 143 g |
| | Toluene: | Stir at | Compound | | | Purity: |
| | 7.5 V | 95 ± 5 for | 102: 11.9 | HCl/EtOH | Compound | 99.4 A % |
| | H₂O (0.2 V) | 16 h | min: 3.8 A % | (3 V) | 102: 11.9 | Yield: |
| | Solution 2 | Solution 2 | INT: 4.5 | Stirred for | min: 56.0 | 43% |
| | SM3: 1.5 | added to | A % | at least 3 h. | A % | |
| | eq. | solution 1 at | 62.0% assay | Concentrated | Compound | Residual |
| | Toluene: | 95 ± 5 C. | yield | to 3-4 V | 101: 17.9 | S: 150 |
| | 7.5 V | for 6 h | 2: 22.4 min: | charged EA | min: 0.2 | ppm |
| | Dioxane: | Stir at 95 ± 5 | 1.0 A % | (6 V) | A % | |
| | 7.5 V | for 16 h | Compound | Charged 4 M | Compound | |
| | Solution 1 | | 101: 17.9 | HCl/EtOH | | |
| | 2: 300 g | | min: 57.5 | (3 V) | 102: 11.9 | |
| | (1.0eq.) | | A % | Stirred for at | min: 58.0 | |
| | NH₄OAc: 5.0 | | Compound | least 3 h. | A % | |
| | eq. | | 102: 11.9 | | | |
| | Toluene: 7.5 | | min: 3.4 A % | | | |
| | V | | INT: 2.3 | | | |
| 9 | H2O (0.2 V) | | A % | | | |
| | | | 61.9% assay | | | |
| | Solution 2 | | yield | | | |
| | SM3: 1.5 eq. | | | | | |
| | Toluene: 7.5 | | | | | |
| | V | | | | | |
| | Dioxane: 7.5 | | | | | |
| | V | | | | | |

300 g scale of demo batch provided 57.5 A % IPC purity in step 3 (assay yield at the end of reaction: 61.9%) and 58.0 A % IPC purity in step 4, after work-up, 143 g of Compound 102 was obtained with 99.4 A % purity and 44% yield (corrected by QNMR).

### Example 47. Kilogram-scale synthesis of 2-(3-bromopyridin-4-yl)-3-((3-chloro-2-methoxyphenyl)amino)-1,5,6,7-tetrahydro-4H-pyrrolo[3,2-c]pyridin-4-one (Compound 102)

### Production Summary

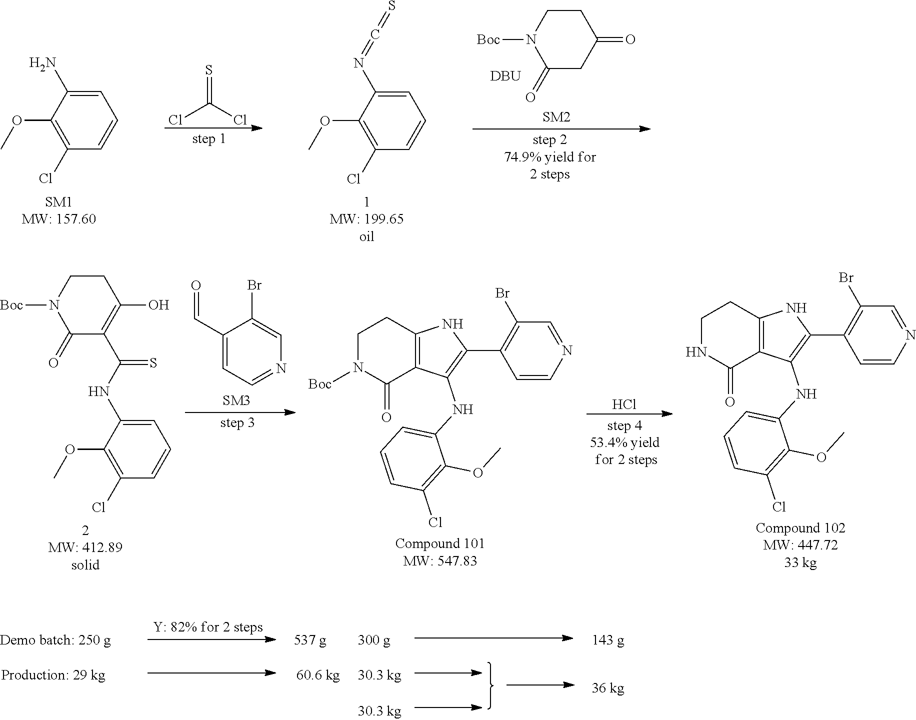

### Production of Step 1 & 2

### 1) Process Route

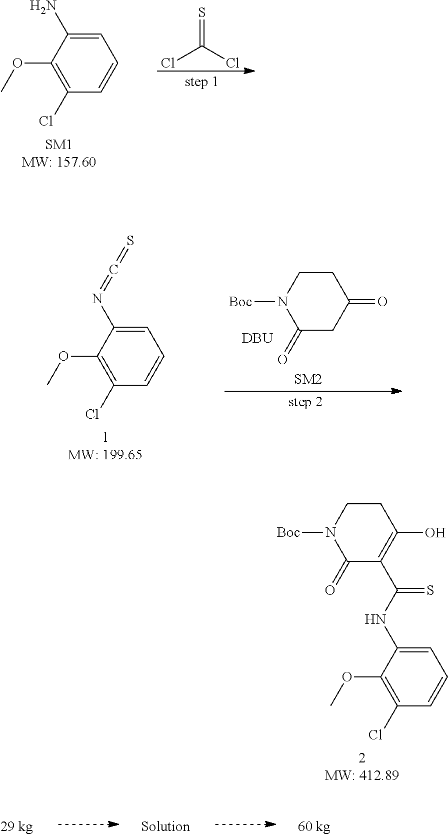

### 2) Process Description

Preparation of 1-chloro-3-isothiocyanato-2-methoxybenzene, 1 Under nitrogen atmosphere, spray isopropyl acetate (IPAC) into the reactor, heat reflux for at least 30 minutes and cool down to 20±10° C., through the feed line, filter tank, pneumatic pump, liquid transfer line, transfer another reactor, heat reflux for at least 30 minutes and cool down to 20±10° C., put bucket. Drying reaction kettle, blow-dry discharge pipeline, pressure filter tank, pneumatic pump, liquid transfer pipeline.

### 1^{st} Separation

Stand for at least 30 minutes under nitrogen, separate, collect the aqueous phase and organic phase.

### 1^{st} Extraction

Under N₂, charge aqueous phase to a reactor, adjust the temperature to 20±5 C. Charge IPAc (3.00 V) to the reactor at 20±5 C, stir for at least 20 minutes, stand for at least 30 minutes, separate, collect the aqueous phase and organic phase. Take sample of aqueous phase for product loss test (IPM), the organic phase waits for combination and washing.

### Washing

Under N₂, charge 10% sodium chloride aqueous solution (3.33 w/w) to reactor with the organic phase, adjust temperature to 20±5 C, stir for at least 20 minutes, stand for at least 30 minutes, separate, collect the organic phase and wait for 1^{st} concentration.

### 1^{st} Concentration

Add the organic phase to the reactor through a fluid filter. Control the reactor inner temperature not more than 45 C or jacket temperature not more than 55 C and concentrate until the volume is 1.0˜1.5 V.

### 2^{nd} Concentration

Under N₂, charge MeOH (3.00 V) to reactor. Control the reactor inner temperature not more than 45° C. or jacket temperature not more than 55° C. and concentrate until the volume is 1.0˜1.5 V

### 3^{rd} Concentration

Under N₂, charge MeOH (3.00 V) to reactor. Control the reactor inner temperature not more than 45 C or jacket temperature not more than 55 C and concentrate until the volume is 1.0˜1.5 V. Adjust temperature to 25±5 C. Charge MeOH (3.00 V) to reactor. Sample for GC analysis. Criterion is: the area % of IPAc≤5% and KF≤0.5%, if the area % of IPAc>5% or KF>0.5%, repeat the solvent exchange procedure with MeOH until the area % of IPAc≤5% and KF≤0.5%. Sample for Q-NMR, report result. Discharge the concentration system in reactor to drum.

### Preparation of 1-chloro-3-isothiocyanato-2-methoxybenzene, 1

### Charging and Reaction

Charge IPAC (20.00 V) to reactor and start agitation under nitrogen. Adjust the temperature to 20±5° C. Take a sample for KF after stirring for at least 5 minutes, criterion: KF is no more than 0.08%. if not, discharge and charge new solvent. Then charge 3-Chloro-2-methoxyaniline (SM1 1.00 eq) and DIPEA (2.50 eq) to the reactor in turn. Cool to 0-5° C. and charge Thiophosgene (0.98 eq) dropwise to the reactor at 5±5° C. (It is recommended to add at least 2 hour). Addition completely, continue to control temperature at 5±5° C., agitate for at least 2 hour. Sample for HPLC analysis, the criterion: the area % of SM1≤3.0% and the total sample times should be no more than two times. Sample for IPM analysis, report content of Thiophosgene.

### Quenched

Under nitrogen and adjust the temperature of hydrochloric acid solution to 5-10 C, replacement with nitrogen three times. Charge reaction system to 3 M hydrochloric acid aqueous (3.00 V) at 10±5 C. Take a sample for pH=1˜4. Stir for at least 30 minutes, retest the pH, criterion is pH=1˜4; if pH>4, charge 3 M hydrochloric acid aqueous solution to make sure the pH=14. Stir for at least 3 hours at 20±5 C (during stirring, pump nitrogen to remove hydrogen in the system).

### 4^{th} Concentration

Control the inner temperature of the reactor not more than 45 C or jacket temperature not more than 55 C and concentrate until the volume is 3-4 V. Adjust temperature to 20±5 C.

### 2^{nd} Separation

Charge soften water (3.00 V) to a reactor at 20±5 C under nitrogen and start to stir. Dropwise 10% sodium carbonate aqueous solution (6.67 w/w) to reaction when temperature 20±5 C. Take a sample for pH=8-9, stir at least for 30 minutes, retest pH=8˜9. If not, charge sodium carbonate into reactor at 20±5 C until pH=8˜9, stir at least for 30 minutes, retest pH=8˜9. Charge IPAc (5.00 V), stir for at least 20 minutes, stand for at least 30 minutes, separate, collect the aqueous phase and organic phase.

### 2^{nd} Extraction

Under N₂, charge aqueous phase to a reactor, adjust the temperature to 20±5 C. Charge IPAc (5.00 V) to the reactor at 20±5 C, stir for at least 20 minutes, stand for at least 30 minutes, separate, collect the aqueous phase and organic phase.

### 3^{rd} Extraction

Under N₂, charge aqueous phase to a reactor, adjust the temperature to 20±5 C. Charge IPAc (5.00 V) to the reactor at 20±5 C, stir for at least 20 minutes, stand for at least 30 minutes, separate, collect the aqueous phase and organic phase. combine the organic phase. Take sample of aqueous phase for product loss test (IPM), the organic phase wait for concentration.

### 5^{th} Concentration

Add the organic phase to the reactor through a fluid filter. Control the inner temperature of the reactor not more than 45 C or jacket temperature not more than 55 C and concentrate until the volume is 1.0˜1.5 V.

6^{th} concentration

Charge DCM (5.00 V) to reactor. Control the inner temperature of the reactor not more than 45 C or jacket temperature not more than 55 C and concentrate until the volume is 1.0˜1.5 V

### 7^{th} Concentration

Charge DCM (5.00 V) to reactor. Control the inner temperature of the reactor not more than 45 C or jacket temperature not more than 55 C and concentrate until the volume is 1.0˜1.5 V Adjust temperature to 25±5 C. Charge DCM (4.00 V) to reactor. Sample for GC analysis. Criterion is: the area % of MeOH≤5% and the area % of TPAc≤20% and KF≤, 0.2%, if the area % of MeOH>5% or IPAc>20% or KF>0.2%, repeat the solvent exchange procedure with DCM until the area % of MeOH≤5% and the area % of IPAc≤20% and KF≤0.2%.

### Feeding (Liquid Product)

Sample for HPLC and Q-NMR test. Report result. Tranfer the product in the reactor into drums, weight and label. Store at room temperature.

### 3) Process of Step 1 & 2

1. Charge IPAC (20 V) to a reactor under nitrogen.
2. Charge SM1 (1.0 eq.) and TEA (2.5 eq.) to the reactor and start to stir at 20±5 C.
3. Cool to 5±5 C.
4. Charge SCCl₂ (1.0 eq.) drop-wise to the reactor at 5±5 C.
5. Stir 2 h at 5±5 C.
6. Sample for HPLC analysis.
7. Filtrate.
8. Charge the IPAC solution and SM2 (1.0 eq.) to the reactor under nitrogen.
9. Cool to 5±5 C.
10. Charge DBU (1.1 eq.) drop-wise to the reactor.
11. Stir for 12 h at 20±5 C.
12. Sample for HPLC analysis.
13. Adjust pH of reaction to 5-6 using aq. citric acid (0.2 M) at 20±5 C.
14. Separate and wash the organic phase with 5% aq. NaHCO₃ (5 V) at 20±5 C.
15. Wash the organic phase with 15% aq. NaCl (5 V) at 20±5 C.
16. Concentrate the organic phase to 3-4 V at 45±5 C.
17. Add MeOH (10 V) and concentrate to 4-5 V at 45±5 C.
18. Add MeOH (10 V) and concentrate to 4-5 V at 45±5 C.
19. Stir for 1 h at 20±5 C.
20. Filter and wash filter the cake with MeOH (2 V)
21. Collect and dry the cake at 40±5 C.

### 4) Production Data Summary

| | Step 1 | | Reaction | | | | Reaction | | |
|---|---|---|---|---|---|---|---|---|---|
| | Starting Materials | | Conditions | | | Step 2 | Conditions | | |
| Lot | SMI | Reagents | Temp./Time | IPC | Work up | Reagents | Temp./Time | IPC | Result |
| Use-test | 20 g | SCCl₂: | 0~10 C. | SM1 | Filtrate | SM2: | 0~25° C. | 1(9.25 min): | N/A |
| | 1 eq. | 1.0 eq. | 3 h | (5.71 min): | Use the | 1.0 eq. | 12 h | 1.1A % | |
| | | DIEA: | | 0.4A %; | filtrate | DBU: | | 2(9.81 min): | |
| | | 2.5eq. | | 1 | to next | 1.1 eq. | | 88.8A % | |
| | | IPAC | | (9.25 min): | step | | | | |
| | | (20 V) | | 97.9A % | directly | | | | |
| Production | 29 kg | SCCl₂: | 0~10 C. | SMI (5.71 | Filtrate | SM2: | 0~25° C. | 1(9.36 min): 2.9 | 60.6 kg |
| | 1 eq. | 1.0 eq. | 3 h | min): 1.2 | Use the | 1.0 eq. | 12 h | A % | Purity: |
| | | DIEA: | | A %; | filtrate | DBU: | | 2(9.92 min): | 99.5% |
| | | 2.5eq. | | 1 | to next | 1.1 eq. | | 85.0A % | Yield: |
| | | IPAC | | | (9.26 min): | step | | | 80% |
| | | (20 V) | | | 97.7A % | directly | | | |

### 5) Results

20 g scale use-test worked well with 97.9 A % IPC purity in step 1 and 88.8 A % IPC purity in step 2.
29 kg of production batch worked well with 97.7 A % IPC purity in step 1 and 85.0 A % IPC purity in step2, after work-up, 60 kg of comp. 2 was obtained with 99.5 A % IPC purity and 78.9% yield (uncorrected by QNMR).

### Production of Step 3 & 4

### 1) Process Route

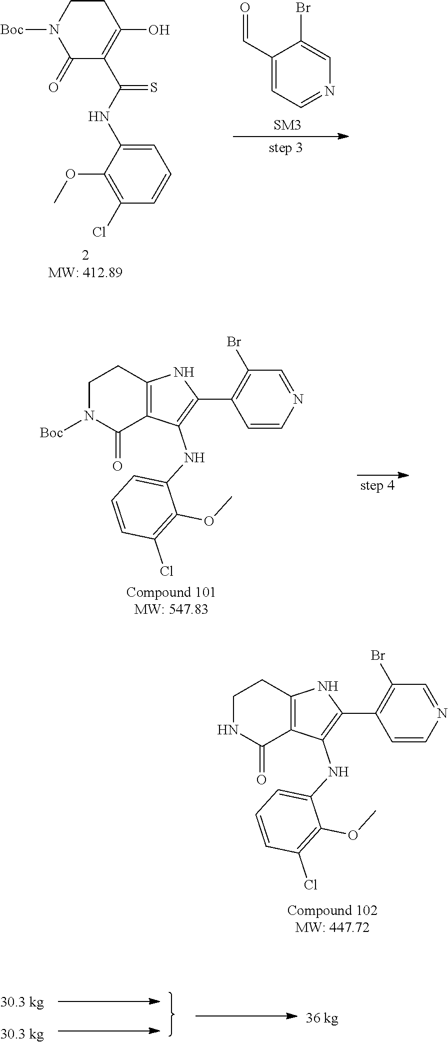 [SMILES: COc1c(Cl)cccc1NC(=S)C1=C(O)CCN(C(=O)OC(C)(C)C)C1=O ; O=Cc1ccncc1Br ; COc1c(Cl)cccc1Nc1c(-c2ccncc2Br)[nH]c2c1C(=O)N(C(=O)OC(C)(C)C)CC2 ; COc1c(Cl)cccc1Nc1c(-c2ccncc2Br)[nH]c2c1C(=O)NCC2]

### 2) Process Description

### Preparation of 6% the Solution of Citric Acid

Under nitrogen, charge soften water (24.00V) to reactor, start agitation. Charge citric acid (1.51 w/w) to the reactor, adjust the temp to 20±10° C., stir for dissolved, discharge into drum for temporary storage.

### Charging and Reaction

Charge IPAC (8.00 V) to reactor which store the solution of 1-chloro-3-isothiocyanato-2-methoxybenzene and start agitation under nitrogen. Adjust the temperature to 20±5° C. Take a sample for KF after stirring for at least 5 minutes, report the result. Then charge 1,8-Diazabicyclo[5.4.0]undec-7-ene (DBU) at 20±5° C. Cool to 0-5° C. and charge Diazabicyclo (1.10 eq) dropwise to the reactor at 5±5° C. (It is recommended to add at least 1 hour). Addition completely, adjust temperature to 20±5° C. (It is recommended that the temperature rise for at least 1 hour), agitate for at least 12 hour. Sample for HPLC analysis, the criterion: the area % of 1-chloro-3-isothiocyanato-2-methoxybenzene (1)≤5.0%. If not, agitate for at least 8 hours, sample for HPLC until area % of 1-chloro-3-isothiocyanato-2-methoxybenzene (1)≤5.0%. Sample for IPM analysis, report content of Thiophosgene.

### Quenching and Separation

Charge 6% citric acid solution to reaction system to adjust PH to 5-6 at 20±5° C. Stir at least 20 mins, test pH=5-6. Stir at least 2 hours at 20±5° C. (System drum nitrogen). Sample for Hydrogen sulfide detected. The hydrogen sulfide concentration in the head space is less than 1 ppm. If it is unqualified, continue to blow nitrogen until it meets the standard. hold at least for 30 mins, separate, collect the organic phase.

### Washing

Charge soften water (5.00V) to organic phase, adjust temperature to 20±5° C., stir at least 30 mins, hold at least 30 mins, separate, collect organic treat concentrate.

### 1^{st} Concentration

Control the jacket temperature is no more than 45° C. and inner temperature is no more than 35° C. Concentration to 5-6V.

### 2^{nd} Concentration

Adjust temperature to 20±10° C., charge methanol (10.00V) into reactor. Control the jacket temperature is no more than 45° C. and inner temperature is no more than 35° C. Concentration to 5-6V.

### 3^{rd} Concentration

Adjust temperature to 20±10° C., charge methanol (10.00V) into reactor. Control the jacket temperature is no more than 45° C. and inner temperature is no more than 35° C. Concentration to 5-6V. Adjust temperature to 20±5° C., sample for HPLC and GC(IPM), Report content of 2 and area % of IPAC in mother liquor.

### Feeding (Liquid Product)

Sample for HPLC and Q-NMR test. Report result. Transfer concentration in the reactor into drums, weight and label. Store at room temperature.

### 3) Process of Step 3 & 4

1. Charged tert-butyl 5-((3-chloro-2-methoxyphenyl)carbamothioyl)-4-hydroxy-6-oxo-3,6-dihydropyridine-1(2H)-carboxylate (2) (1.0 eq.) and NH₄OAc (5.0 eq.) to toluene (7.5 V).
2. Heated to 95±5 C and charged pre-prepared solution of SM3 (1.5 eq. in toluene/1,4-dioxane 15 V, 1/1) drop-wise to the reactor at 95±5 C.
3. Stirred for 16 h at 95±5 C and sampled for HPLC analysis, criterion: tert-butyl 5-((3-chloro-2-methoxyphenyl)carbamothioyl)-4-hydroxy-6-oxo-3,6-dihydropyridine-1(2H)-carboxylate (2)≤5.0%.
4. Concentrated to 3-4 V at 50±5 C and charged EA (6 V) to the reactor.
5. Charged 4 M HCl/EtOH (3 V) drop-wise to the reactor at 25±5 C and stirred for at least 3 h.
6. Sampled for HPLC analysis, criterion: tert-butyl 2-(3-bromopyridin-4-yl)-3-((3-chloro-2-methoxyphenyl)amino)-4-oxo-1,4,6,7-tetrahydro-5H-pyrrolo[3,2-c]pyridine-5-carboxylate (compound 101)≤2.0%.
7. Filtered and washed the cake with EA (1 V).
8. Charged filter cake to EA/MeOH=10:1 (10 V).
9. Charged 10% K₂CO₃ aq. (10 V) to the reactor at 25±5 C and stirred for at least 5 h. (Note: pH: 8˜9)
10. Filtered and washed the cake with MTBE (1 V) and dried for 16 h at 50 C under N₂.
11. Changed 1 V of DMAc and 3 V of toluene to a reactor and charged crude (compound 102) to the reactor.
12. Heated to 75±5 C and stirred for at least 4 h.
13. Cooled to 45±5 C and charged 5 V of toluene drop-wise to the reactor.
14. Stirred for at least 2 h at 45±5 C.
15. Cool to 10±5 C and stir for at least 3 h at 10±5 C.
16. Filtered and washed with toluene (1 V).
17. Changed 10 V of water and wet cake to a reactor.
18. Heated to 50±5 C and stirred for at least 3 h at 50±5 C.
19. Cooled to 10±5 C and stirred for at least 3 h at 10±5 C.
20. Filtered and washed with water (1-2 V).
21. Dried for 16 h at 50±5 C under N₂.

### Production Data Summary

| | | | | | Step 4: | | |
|---|---|---|---|---|---|---|---|
| | | | | | Starting | | |
| | | | Reaction | | Materials | | |
| | Step 3: | | Conditions | | Reaction | | |
| Lot | Starting Materials | | Temp./Time | IPC | Conditions | IPC | Result |
| Use | Solution 12: | Solution | Solution 2 | 2: 1.6 A % | N/A | N/A | N/A |
| test | 15 g (1.0 | 2 | added to | Compound 101: | | | |
| | eq.) | SM3: | solution 1 at | 59.6 A % | | | |
| | NH₄OAc: | 1.5 eq. | 95 ± 5 C. for | Compound 102: | | | |
| | 5.0 eq. | Toluene: | 6 h | 2.6 A % | | | |
| | Toluene: | 7.5 V | Stir at 95 ± 5 | INT: 1.1 A % | | | |
| | 7.5 V | Dioxane: | for 16 h | 62.1% assayyield | | | |
| | H₂O (0.2 | 7.5 V | | | | | |
| | V) | | | | | | |
| Batch | Solution 12: | Solution | Solution 2 | 2: 0.6 A % | Concentrated | Compound | 36 kg |
| 1 | 30.3 kg | 2 | added to | Compound 101: | to 3-4 V | 101: 0.7 A % | Purity: 99.0 A % |
| | (1.0 eq.) | SM3: | solution 1 at | 57.9 A % | Charged | Compound | Yield: 54.8% |
| | NH₄OAc: | 1.5 eq. | 95 ± 5 C. for 6 h | Compound 102: | EtOAc (6 V) | 102: 63.1 | (uncorrected by |
| | 5.0 eq. | Toluene: | Stir at 95 ± 5 | 5.3 A % | Charged 4M | A % | QNMR) |
| | Toluene: | 7.5 V | for 16 h | INT: 1.2A % | HCl/McOH | | |
| | 7.5 V | Dioxane: | | Assay yield: | (3 V) | | |
| | H₂O (0.2 | 7.5 V | | being collected | Stirred for at | | |
| | V) | | | | least 3 h | | |
| Batch | Solution 1 | | Solution 2 | 2: 0.9 A % | | | |
| 2 | 2: 30 kg | Solution | added to | Compound 101: | | | |
| | (1.0 eq.) | 2 | solution 1 at | 60.1 A % | | | |
| | NH₄OAc: | SM3: | 95 ± 5 C. for 6 | Compound 102: | | | |
| | 5.0 eq. | 1.5 eq. | h | 4.4 A % | | | |
| | Toluene: | Toluene: | Stir at 95 ± 5 | INT: 1.7 A % | | | |
| | 7.5 V | 7.5 V | for 16 h | Assay yield: | | | |
| | H₂O (0.2 | Dioxane: | | 62.5% | | | |
| | V) | 7.5 V | | | | | |

### 4) Results

The first 30.3 kg scale of production batch worked well with 57.9 A % IPC purity in step 3, the assay yield of compound 101 at the end of reaction was 61.5%.
The second 30.3 kg scale of production batch worked well with 60.1 A % IPC purity in step 3, the assay yield of compound 101 at the end of reaction was 62.5%.
After concentrating separately, the two batches were combined for step 4 reaction directly. The step 4 reaction worked well with 63.1 A % IPC purity.
After work-up and purification, 36 kg of compound 102 was obtained with 99.0 A % purity (toluene wasn't integrated) and 54.8% yield (uncorrected by QNMR).

### Example 48. Synthesis of tert-Butyl 3-((3-chloro-2-methoxyphenyl)amino)-4-oxo-2-(pyridin-4-yl)-1,4,6,7-tetrahydro-5H-pyrrolo[3,2-c]pyridine-5-carboxylate

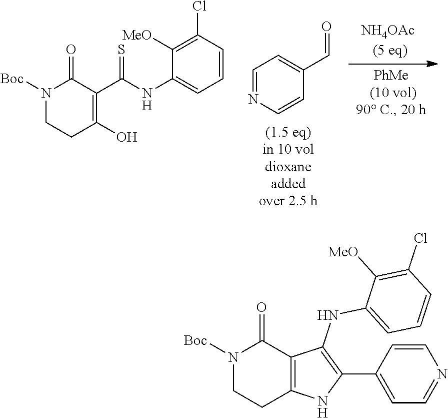

A solution of isonicotinaldehyde (1.71 mL, 18.2 mmol, 1.5 eq) in 1,4-dioxane (50 mL) was added dropwise over 2.5 hours with a syringe pump to a preheated, stirred, solution of tert-butyl 5-((3-chloro-2-methoxyphenyl)carbamothioyl)-4-hydroxy-6-oxo-3,6-dihydropyridine-1(2H)-carboxylate (5.00 g, 12.1 mmol, 1.0 eq) and NH₄OAc (4.67 g, 60.6 mmol, 5.0 eq) in toluene (50 mL) at 90° C. The reaction mixture was stirred at 90° C. for 20 hours and then concentrated under reduced pressure. The crude residue was adsorbed onto silica and then purified by silica gel chromatography (25-100% heptane/EtOAc and then 0-10% MeOH in DCM) to afford tert-butyl 3-((3-chloro-2-methoxyphenyl)amino)-4-oxo-2-(pyridin-4-yl)-1,4,6,7-tetrahydro-5H-pyrrolo[3,2-c]pyridine-5-carboxylate (3.36 g, 59% yield) as an orange solid.

LCMS (ES, m/z): [M+H]⁺: 469.2;

¹H NMR (400 MHz, DMSO-d₆) δ 12.14 (s, 1H), 8.45 (d, J=5.0 Hz, 2H), 7.47 (d, J=4.9 Hz, 2H), 7.36 (s, 1H), 6.73 (d, J=7.5 Hz, 2H), 6.16 (dt, J=7.1, 2.1 Hz, 1H), 3.97 (t, J=6.3 Hz, 2H), 3.91 (s, 3H), 2.98 (t, 1=6.3 Hz, 2H), 1.44 (s, 9H);

¹³C NMR (101 MHz, DMSO-d₆) δ 162.3, 153.2, 150.3, 143.7, 140.7, 140.1, 138.3, 127.0, 125.6, 125.4, 122.3, 118.9, 118.8, 112.2, 109.8, 81.9, 60.2, 45.6, 28.2, 22.8.

### REFERENCES

1. Graham, K.; Klar, U.; Briem, H.; Schulze, V.; Siemeister, G.; Lienau, P.; Tempel, R.; Balint, J. 4H-Pyrrolo[3,2-c]pyridin-4-one Derivatives. WO2016/120196 A1, 2016.
2. Siegel, F.; Korr, D.; Schroder, J.; Siegel, S.; Greulich, H.; Kaplan, B.; Meyerson, M. 4H-Pyrrolo[3,2-c]pyridin-4-one Derivatives. WO2020/216774 A1, 2020.
3. Jagodzinski, T. S.; Sosnicki, J. G.; Struk, L. ARKIVOC 2017, 5, 43-57.
4. Siegel, S.; Siegel, F.; Schulze, V.; Berger, M.; Graham, K.; Klar, U.; Eis, K.; Sülzle, D.; Bomer, U.; Korr, D.; Peterson, K.; Mönning, U.; Eberspacher, U.; Moosmayer, D.; Meyerson, M.; Greulich, H.; Kaplan, B.; Harb, H. Y.; Dinh, P. M. 4H-Pyrrolo[3,2-c]pyridin-4-one Derivatives. WO2019/081486 A1, 2019.
5. Milgram, B. C.; White, R. D.; St. Jean Jr., D.; Guzman-Perez, A. Pyrrolo[3,2-c]pyridin-4-one Derivatives Useful in the Treatment of Cancer. WO2022/066734 A1, 2022.

What is claimed is:

1. A method of preparing a compound of Formula (I), or a pharmaceutically acceptable salt thereof, the method comprising contacting a compound of formula (II) with a compound of formula (III), 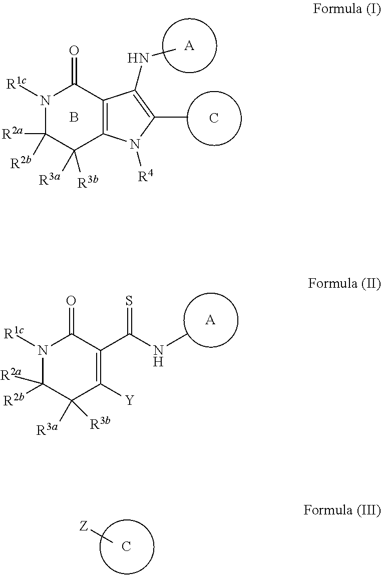
wherein
Y is selected from —OH or —NH₂;
Z is selected from
—C(═O)H; or
—CH(R)₂ wherein each R independently selected from halo, alkoxy, OH or SO₃M (M=Li, Na, K or NH₄⁺), provided that when one R is OH, the other R cannot be halo, alkoxy or OH;
R^{1c} is selected from H, and R^{d};
each of R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g};
two of variables R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})— when —N(R^{1c})—forms part of the fused saturated or unsaturated ring), wherein each of the independently selected heteroatoms is selected from the group consisting of N, NH, N(R^{d}), 0, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d};
Ring C is selected from the group consisting of: 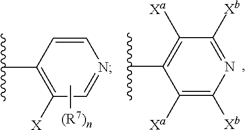
wherein:
each X^{b} is independently X, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X and further optionally substituted with from 1-4 R^{c};
2-pyridonyl or 4-pyridonyl, each optionally substituted with X and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷;
bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷;
heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷; and
C₁₀ or C₁₄ aryl optionally substituted with X and optionally substituted with from 1-4 R⁷; 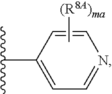
wherein
ma is 0, 1, 2, or 3;
R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A};
R^{5A} is selected from hydroxy, halogen, cyano, C₁₋₄-alkyl, C₁₋₄-alkoxy, C₁₋₄ haloalkyl or C₁₋₄ haloalkoxy;
R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{5A};
R^{11A} is independently selected from C₁₋₄ alkyl, C₃₋₆ cycloalkyl, C₁₋₄ haloalkyl or C₃₋₆ halocycloalkyl; 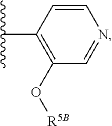
wherein
R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{5A}; or
R^{5B} is R^{6B}—CH₂—;
R^{6B} is selected from 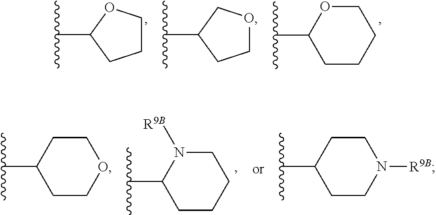
R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy; 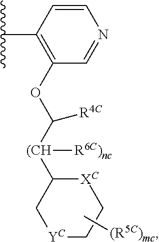
wherein
R^{4C} is selected from hydrogen or methyl;
R^{6C} is selected from hydrogen, C₁₋₃ alkyl, C₁₋₃ haloalkyl;
nc is 0 or 1;
X^{C} is NR^{7C} or O;
Y^{C} is NR^{8C} or O;
R^{7C} is methyl;
R^{8C} is selected from methyl, 2,2,2-trifloethyl, or 2,2-difluoroethyl;
R^{5C} is selected from hydrogen or methyl, wherein Rec being attached to any carbon atom of the ring comprising X^{C} and Y^{C};
mc is 0, 1, 2, or 3; 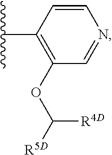
wherein
R^{4D} is selected from hydrogen or methyl;
R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R^{6D} and wherein each azetidinyl is substituted at the nitrogen with R^{8D}; OR
R^{5D} is selected from the group consisting of 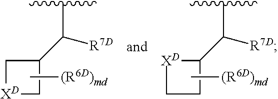
R^{6D} is selected from fluoro or C₁₋₃ alkyl;
md is selected from 0, 1, 2, or 3;
R^{7D} is selected from hydrogen, C₁₋₃ alkyl, or C₁₋₃ haloalkyl;
X^{D} is NR^{8D} of O;
R^{8D} is selected from C₁₋₃ alkyl or C₂₋₃ haloalkyl;
X is X*, wherein X* is selected from halo, triflate, tosylate, or mesylate; or
X is X¹;
each R⁷ is an independently selected R^{c}; n is 0, 1, 2, or 3;
X¹ is selected from the group consisting of: (a) —O-L¹-R⁵; and (b) 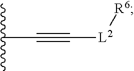
L¹ and L² are independently selected from the group consisting of: a bond and C₁₋₁₀ alkylene optionally substituted with from 1-6 R^{a};
R⁵ is selected from the group consisting of:
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c};
C₆₋₁₀ aryl optionally substituted with from 1-4 R^{c};
C₃₋₁₀ cycloalkyl or C₃₋₁₀ cycloalkenyl, each optionally substituted with from 1-4 substituents each independently selected from the group consisting of: oxo and R^{c}; 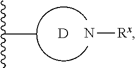
wherein Ring D is heterocyclylene or heterocycloalkenylene including from 3-10 ring atoms, wherein from 0-2 ring atoms (in addition to the ring nitrogen atom bonded to R^{X}) are heteroatoms, each independently selected from the group consisting of N, N(H), N(R^{d}), O, and S(O)₀₋₂, and wherein the heterocyclylene or heterocycloalkenylene is optionally substituted with from 1-4 substituents each independently selected from the group consisting of: oxo and —R^{c};
—S(O)₀₋₂(C₁₋₆ alkyl) which is optionally substituted with from 1-6 R^{a},
—R^{W}
—R^{g2}—R^{W} or —R^{g2-}R^{Y};
-L⁵⁻R^{g}; and
-L⁵-R^{g2}—R^{W} or -L⁵-R^{g2}—R^{Y};
provided that when L¹ is a bond, then R⁵ is other than —S(O)₀₋₂(C₁₋₆ alkyl) which is optionally substituted with from 1-6 R^{a}; -L⁵-R^{g}; -L⁵-R^{g2}—R^{W}; or -L⁵-R^{g2}—R^{Y};
R⁶ is selected from the group consisting of:
H;
halo;
—OH;
—NR^{e}R^{f};
—R^{g};
—R^{W}
-L⁶-R^{g};
—R^{g2}—R^{W} or —R^{g2}—R^{Y};
-L⁶-R^{g2}—R^{W} or -L⁶-R^{g2}—R^{Y}; and
—C₁₋₆ alkoxy or —S(O)₀₋₂(C₁₋₆ alkyl), each optionally substituted with from 1-6 R^{a};
L⁵ and L⁶ are independently —O—, —S(O)₀₋₂, —NH, or —N(R^{d})—; and
R^{W} is -L^{W}-W,
wherein L^{W} is C(═O), S(O)₁₋₂, OC(═O)*, NHC(═O)*, NR^{d}C(═O)*, NHS(O)₁₋₂*, or NR^{d}S(O)₁₋₂*, wherein the asterisk represents point of attachment to W, and
W is C₂₋₆ alkenyl; C₂₋₆ alkynyl; or C₃₋₁₀ allenyl, each of which is optionally substituted with from 1-3 R^{a} and further optionally substituted with R^{g}, wherein W is attached to L^{W} via an sp² or sp hybridized carbon atom, thereby providing an α, β-unsaturated system; and
R^{X} is C(═O)(C₁₋₆ alkyl) or S(O)₂(C₁₋₆ alkyl), each of which is optionally substituted with from 1-6 R^{a}; and
R^{Y} is selected from the group consisting of: —R^{g} and -(L^{g})_{g}-R^{g}.
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*;
C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c};
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

2. The method of claim 1, wherein contacting the compound of formula (II) with the compound of formula (III) is carried out in the presence of a nitrogen source.

3. The method of claim 2, wherein the nitrogen source is ammonia or derivative thereof.

4. The method of claim 3, wherein the nitrogen source is in the form of a salt.

5. The method any one of claims 2-4, wherein the nitrogen source is selected from NH₄OAc, NH₃·H₂O, NH₄CO₂H, NH₄OBz, NH₄Cl, (NH₄)₂SO₄, (NH₄)₂HPO₄, NH₄H₂PO₄, NH₄OTf, NH₄HCO₃, (NH₄)₂CO₃, NH₄CO₂CF₃, NH₄BF₄, ammonium citrate dibasic, (C₁-C₆ alkyl)-NH₂, and (C₃-C₆ cycloalkyl)-NH₂, or any combination thereof.

6. The method of any one of claims 2-5, wherein the nitrogen source is NH₄OAc.

7. The method of any one of claims 2-6, wherein the molar ratio of the nitrogen source to the compound of formula (III) is from about 2:1 to about 8:1.

8. The method of any one of claims 2-6, wherein the molar ratio of the nitrogen source to the compound of formula (III) is from about 4:1 to about 6:1.

9. The method of any one of claims 2-6, wherein the molar ratio of the nitrogen source to the compound of formula (III) is about 5:1.

10. The method of any one of claims 1-9, wherein contacting the compound of formula (II) with the compound of formula (III) is carried out in the presence of a solvent.

11. The method of claim 10, where the solvent is an aprotic solvent.

12. The method of claim 11, wherein the aprotic solvent is a non-polar aprotic solvent.

13. The method of claim 12, wherein the non-polar aprotic solvent is an aromatic hydrocarbon solvent.

14. The method of claim 13, wherein the aromatic hydrocarbon solvent is toluene.

15. The method of claim 12, wherein the non-polar aprotic solvent is a non-aromatic hydrocarbon.

16. The method of claim 13, wherein the non-aromatic hydrocarbon is heptane or hexane.

17. The solvent of claim 11, where the aprotic solvent is a polar aprotic solvent.

18. The solvent of claim 17, wherein the aprotic solvent is an ethereal solvent.

19. The solvent is claim 18, wherein the ethereal solvent is CPME, 1,4-dioxane, or THF.

20. The solvent of claim 17, wherein the aprotic solvent is acetonitrile, or DMSO.

21. The method of claim 10, wherein the solvent is a protic solvent.

22. The method of claim 21, wherein the solvent is a polar protic solvent.

23. The method of claim 21 or 22, wherein the solvent is acetic acid.

24. The method of any one of claims 1-23, wherein contacting the compound of formula (II) with the compound of formula (III) is carried out at a temperature of from about 80° C. to 110° C.; or from about 80° C. to 100° C. (e.g., 90° C.); or from about 90° C. to 110° C. (e.g., 100° C.).

25. The method of any one of claims 1-24, wherein contacting the compound of formula (II) with the compound of formula (III) is carried out at a temperature of from about 80° C. to 110° C.

26. The method of any one of claims 1-24, wherein contacting the compound of formula (II) with the compound of formula (III) is carried out at a temperature of from about 80° C. to 100° C. (e.g., 90° C.).

27. The method of any one of claims 1-24, wherein contacting the compound of formula (II) with the compound of formula (III) is carried out at a temperature of from about 90° C. to 110° C. (e.g., 100° C.).

28. The method of any one of claims 1-23, wherein contacting the compound of formula (II) with the compound of formula (III) is carried out at a temperature of from about 20° C. to about 80° C. (e.g., 20° C.).

29. The method of any one claims 1-28, wherein the contacting the compound of formula (II) with the compound of formula (III) is carried out in the presence of an additive.

30. The method of any one of claims 1-28 or 29, wherein the additive is selected from Na₂SO₄, H₂O, H₂SO₄, acetic acid, formic acid, Bi(OTf)₃, PPh₃, NH₄OH, NH₄OAc, PPTS, PTSA, pyridine or any combination thereof.

31. The method of the any one of claims 1-30, wherein the contacting the compound of formula (II) with the compound of formula (III) is carried out in a sealed container filled with air.

32. The method of the any one of claims 1-30, wherein the contacting the compound of formula (II) with the compound of formula (III) is carried out in a sealed container filled with inert gas (e.g., nitrogen).

33. The method of the any one of claims 1-30, wherein the contacting the compound of formula (II) with the compound of formula (III) is carried out in an open container.

34. The method of the any one of claims 1-30, wherein the contacting the compound of formula (II) with the compound of formula (III) is carried out in an open container connected to an inert gas (e.g., nitrogen) manifold.

35. The method of any one of claims 1-31, wherein the molar ratio of the compound of formula (II) formula to the compound of formula (III) is from about 1:1 to about 1:3.

36. The method of any one of claims 1-35, wherein the molar ratio of the compound of formula (II) formula to the compound of formula (III) is about 1:1 to about 1:2.

37. The method of any one of claims 1-35, wherein the molar ratio of the compound of formula (II) formula to the compound of formula (III) is about 1:1.3, or about 1:1.5, or about 1:2.

38. The method of any one of claims 1-37, wherein the compound of formula (III) is added portion wise to the reaction.

39. The method of any one of claims 1-38, wherein the method further comprises contacting a compound of formula (IIa) with a compound of formula (IIb) to provide the compound of formula (II):

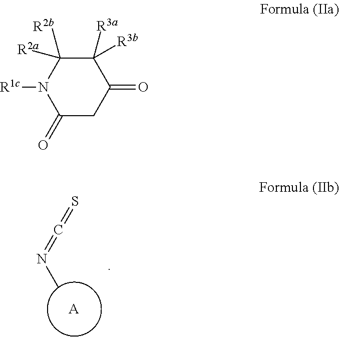

40. The method of claim 39, wherein the compound of formula (IIb) is prepared by contacting a chlorinating agent with a compound of formula (IId):

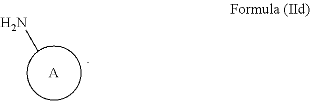

41. The method of any one of claim 38 or 39, wherein the compound of formula (IIb) is prepared by contacting a compound of formula (IIc) with a compound of formula (IId):

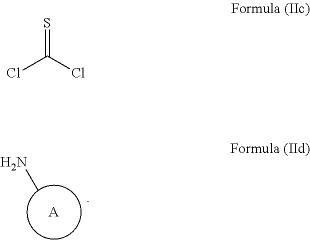

42. The method of any one of claims 1-41, wherein contacting the compound of formula (II) with the compound of formula (III) is carried out in the absence of an oxidizing agent.

43. The method of claim 42, wherein the oxidizing agent is m-CPBA.

44. The method of any one of claims 1-43, wherein the compound of formula (I) is isolated/purified by column chromatography.

45. The method of any one of claims 1-44, wherein R^{1c} is a protecting group.

46. The method of any one of claims 1-44, wherein R^{1c} together with the nitrogen atom to which it is attached forms a carbamate.

47. The method of any one of claims 1-45, wherein R″ is a Boc group.

48. The method of any one of claims 45-47, wherein the method further comprises removing the protecting group from the compound of formula (I).

49. The method of any one of claims 1-44, wherein R″ is H.

50. The method of any one of claims 1-49, wherein each of R^{2a}, R^{2b}, R^{3a}, and R^{3b} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}.

51. The method of any one of claims 1-50, wherein one of R^{2a}, R^{2b}, R^{3a}, and R^{3A} is independently selected from the group consisting of: halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g}; and the other of R^{2a}, R^{2b}, R^{3a}, and R^{3b} is H.

52. The method of any one of claims 1-49, wherein two of variables R^{2a}, R^{2b}, R^{3a}, and R^{3b}, together with the Ring B ring atoms to which each is attached, form a fused saturated or unsaturated ring of 3-12 ring atoms.

53. The method of any one of claims 1-50, wherein each of R^{2a}, R^{2b}, R^{3a}, and R^{3b} is H.

54. The method of any one of claims 1-53, wherein ring A is C₆₋₁₀ aryl optionally substituted with from 1-4 R^{c}.

55. The method of any one of claims 1-54, wherein ring A is phenyl optionally substituted with from 1-4 R^{c}.

56. The method of any one of claims 1-55, wherein ring A is phenyl substituted with from 1-2 R^{c}.

57. The method of any one of claims 1-56, wherein Ring C is

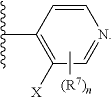

58. The method of any one of claims 1-56, wherein Ring C is 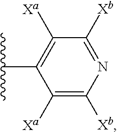
wherein:
each X^{b} is independently X, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅.

59. The method of any one of claims 1-56, wherein Ring C selected from
2-pyridyl or 3-pyridyl, each optionally substituted with X and further optionally substituted with from 1-4 R^{c}; or
2-pyridonyl or 4-pyridonyl, each optionally substituted with X and further optionally substituted with from 1-4 R^{c}, wherein the ring nitrogen atom is optionally substituted with R^{d}.

60. The method of any one of claims 1-56, wherein Ring C is heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c}.

61. The method of any one of claims 1-56, wherein Ring C is heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c}.

62. The method of any one of claims 1-56, wherein Ring C is bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷;

63. The method of any one of claims 1-56, wherein Ring C is bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷.

64. The method of any one of claims 1-56, wherein Ring C is heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷.

65. The method of any one of claims 1-56, wherein Ring C is C₁₀ or C₁₄ aryl optionally substituted with X and optionally substituted with from 1-4 R⁷.

66. The method of any one of claims 1-56, wherein Ring C is selected from the group consisting of: 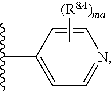
wherein
ma is 0, 1, 2, or 3;
R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A};
R^{5A} is selected from hydroxy, halogen, cyano, C₁₋₄-alkyl, C₁₋₄-alkoxy, C₁₋₄ haloalkyl or C₁₋₄ haloalkoxy;
R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{5A};
R^{11A} is independently selected from C₁₋₄ alkyl, C₃₋₆ cycloalkyl, C₁₋₄ haloalkyl or C₃₋₆ halocycloalkyl; 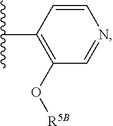
wherein
R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{5A}; or
R^{5B} is R^{6B}—CH₂—;
R^{6B} is selected from 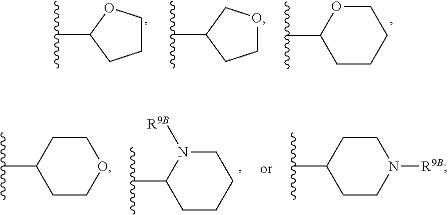
R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy; 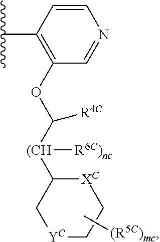
wherein
R^{4C} is selected from hydrogen or methyl;
R^{6C} is selected from hydrogen, C₁₋₃ alkyl, C₁₋₃ haloalkyl;
nc is 0 or 1;
X^{C} is NR⁷C or O;
Y^{C} is NR^{8C} or O;
R^{7C} is methyl;
R^{8C} is selected from methyl, 2,2,2-trifloethyl, or 2,2-difluoroethyl;
R^{5C} is selected from hydrogen or methyl, wherein R^{5C} being attached to any carbon atom of the ring comprising X^{C} and Y^{C};
mc is 0, 1, 2, or 3; 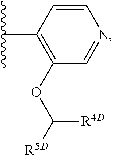
wherein
R^{4D} is selected from hydrogen or methyl;
R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R^{6D} and wherein each azetidinyl is substituted at the nitrogen with R^{8D}; OR
R^{5D} is selected from the group consisting of 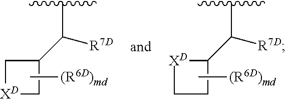
R^{6D} is selected from fluoro or C₁₋₃ alkyl;
md is selected from 0, 1, 2, or 3;
R^{7D} is selected from hydrogen, C₁₋₃ alkyl, or C₁₋₃ haloalkyl;
X^{D} is NR^{8D} of O;
R^{8D} is selected from C₁₋₃ alkyl or C₂₋₃ haloalkyl;

67. The method of any one of claims 1-66, wherein X is halo.

68. The method of any one of claims 1-67, wherein X is bromo.

69. The method of any one of claims 1-66, wherein X is X¹.

70. The method of any one of claims 1-66 or 69, wherein X is —O-L¹-R⁵.

71. The method of any one of claims 1-66 or 69, wherein X is

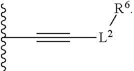

72. The method of any one of claims 1-71, wherein each occurrence of R⁷ is H.

73. The method of any one of claims 1-72, wherein R⁴ is H.

74. The method of any one of claims 1-72, wherein the method further comprises converting the compound of formula (I) wherein X is X* to a compound of formula (I) wherein X is X¹.

75. A compound of Formula (I), or a pharmaceutically acceptable salt thereof, prepared by a process as claimed in any one of claims 1-74:

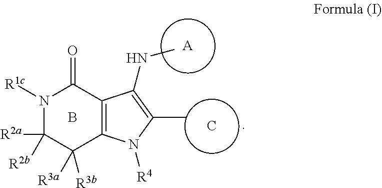

76. A compound of Formula (I), or a pharmaceutically acceptable salt thereof: 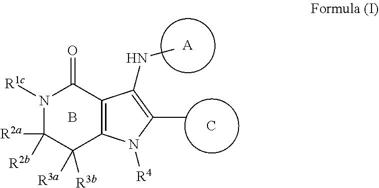
wherein:
R^{1c} is H, —R^{d} or a protecting group (e.g., Boc group);
each of R^{2a}, R^{2b}, R^{3a}, and R^{3A} is independently selected from the group consisting of: H; halo; —OH; —C(O)OH or —C(O)NH₂; —CN; —R^{b}; -L^{b}-R^{b}; —C₁₋₆ alkoxy or —C₁₋₆ thioalkoxy, each optionally substituted with from 1-6 R^{a}; —NR^{e}R^{f}; —R^{g}; and -(L^{g})_{g}-R^{g};
two of variables R^{2a}, R^{2b}, R^{3a}, and R^{3b}, together with the Ring B ring atoms to which each is attached, form a fused saturated or unsaturated ring of 3-12 ring atoms;
wherein from 0-2 of the ring atoms are each an independently selected heteroatom (in addition to —N(R^{1c})—when —N(R^{1c})— forms part of the fused saturated or unsaturated ring), wherein each of the independently selected heteroatoms is selected from the group consisting of N, NH, N(R^{d}), O, and S(O)₀₋₂; and
wherein the fused saturated or unsaturated ring of 3-12 ring atoms is optionally substituted with from 1-4 substituents independently selected from the group consisting of oxo, R^{c}, and R^{W};
Ring A is R^{g};
R⁴ is selected from the group consisting of: H and R^{d};
Ring C is selected from the group consisting of: 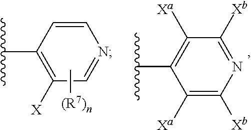
wherein:
each X^{b} is independently X, R^{c}, or H; and
each X^{a} is independently selected from the group consisting of: H, halo; cyano; C₁₋₁₀ alkyl which is optionally substituted with from 1-6 independently selected R^{a}; C₂₋₆ alkenyl; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
2-pyridyl or 3-pyridyl, each optionally substituted with X and further optionally substituted with from 1-4 R^{c};
2-pyridonyl or 4-pyridonyl, each optionally substituted with X and further optionally substituted with from 1-4 R^{c}, wherein the ring nitrogen atom is optionally substituted with R^{d};
heteroaryl including 6 ring atoms, wherein from 2-4 ring atoms are heteroatoms, each independently selected from the group consisting of N, N(H), and N(R^{d}), and wherein the heteroaryl is optionally substituted with X and further optionally substituted with from 1-4 R^{c};
heteroaryl including 5 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and further optionally substituted with from 1-4 R^{c};
bicyclic heteroaryl including 7-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with X and optionally substituted with from 1-4 substituents independently selected from the group consisting of oxo and R⁷;
bicyclic C₅₋₁₀ cycloalkyl or C₅₋₁₀ cycloalkenyl, each of which is optionally substituted with X and is optionally substituted with from 1-4 substituents independently selected from the group consisting of oxo and R⁷;
heterocyclyl or heterocycloalkenyl including from 5-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with X and optionally substituted with from 1-4 substituents independently selected from the group consisting of oxo and R⁷; and
C₁₀ or C₁₄ aryl optionally substituted with X and optionally substituted with from 1-4 R⁷;
X is X*, wherein X* is a selected from halo, triflate, tosylate or mesylate;
each R⁷ is an independently selected R^{c}; n is 0, 1, 2, or 3;
each occurrence of R^{a} is independently selected from the group consisting of: —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} is independently C₁₋₆ alkyl, C₂₋₆ alkenyl, or C₂₋₆ alkynyl, each of which is optionally substituted with from 1-6 R^{a};
each occurrence of L^{b} is independently C(═O); C(═O)O; S(O)₁₋₂; C(═O)NH*;
C(═O)NR^{d}*; S(O)₁₋₂NH*; or S(O)₁₋₂N(R^{d})*, wherein the asterisk represents point of attachment to R^{b};
each occurrence of R^{c} is independently selected from the group consisting of: halo; cyano; C₁₋₁₀ alkyl which is optionally substituted with from 1-6 independently selected R^{a}; C₃₋₅ cycloalkyl; C₂₋₆ alkenyl; C₂₋₆ alkynyl; C₁₋₄ alkoxy optionally substituted with C₁₋₄ alkoxy or C₁₋₄ haloalkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —C(═O)OH; —C(═O)NR′R″; and —SF₅;
each occurrence of R^{d} is independently selected from the group consisting of: C₁₋₆ alkyl optionally substituted with from 1-3 independently selected R^{a} or R^{g}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₃₋₅ cycloalkyl optionally substituted with from 1-3 C₁₋₃ alkyl group; heterocyclyl including from 3-6 ring atoms, wherein from 1-3 ring atoms are heteroatoms, each independently selected from the group consisting of N, N(H), N(R^{d}), O, and S(O)₀₋₂ optionally substituted with from 1-4 substituents independently selected from the group consisting of oxo and R^{c}; C₁₋₆ alkyl optionally substituted with from 1-3 substituents each independently selected from the group consisting of NR′R″, —OH, C₁₋₆ alkoxy, C₁₋₆ haloalkoxy, and halo; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{g} is independently selected from the group consisting of:
C₃₋₁₀ cycloalkyl or C₃₋₁₀ cycloalkenyl, each of which is optionally substituted with from 1-4 substituents independently selected from the group consisting of oxo and R^{c};
heterocyclyl or heterocycloalkenyl including from 3-10 ring atoms, wherein from 1-3 ring atoms are heteroatoms, each independently selected from the group consisting of N, N(H), N(R^{d}), O, and S(O)₀₋₂, and wherein the heterocyclyl or heterocycloalkenyl is optionally substituted with from 1-4 substituents independently selected from the group consisting of oxo and R^{c};
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl is optionally substituted with from 1-4 R^{c}; and
C₆₋₁₀ aryl optionally substituted with from 1-4 R^{c}; 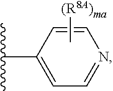
wherein
ma is 0, 1, 2, or 3;
R^{8A} is independently selected from halogen, hydroxy, nitro, cyano, C₁₋₄ alkyl, C₁₋₄ haloalkyl, C₁₋₆ alkoxy, C₃₋₆ cycloalkyl, C₃₋₆ halocycloalkyl, R^{9A}R^{10A}N—, R^{11A}—C(O)—NH—, R^{11A}O—C(O)—NH— or R^{9A}R^{10A}N—C(O)—NH—, wherein said C₁₋₆ alkoxy is optionally substituted one, two or three times, independently of each other, with halogen and is optionally substituted one time with hydroxy, C₁₋₄ alkoxy, R^{9A}R^{10A}N—, C₃₋₆ cycloalkyl, 4-to-7-membered heterocycloalkyl or phenyl, which is optionally substituted one or more R^{5A};
R^{5A} is selected from hydroxy, halogen, cyano, C₁₋₄-alkyl, C₁₋₄-alkoxy, C₁₋₄ haloalkyl or C₁₋₄ haloalkoxy;
R^{9A} and R^{10A} are each independently selected from hydrogen, C₁₋₄ alkyl, C₃₋₆ cycloalkyl, C₁₋₄ haloalkyl, C₃₋₆ halocycloalkyl or phenyl, wherein said phenyl group is optionally substituted, one or more times, independently of each other, with R^{5A}; or R^{9A} and R^{10A} together with the nitrogen atom to which they are attached form a 3- to 6-membered nitrogen containing heterocyclic ring, optionally containing one additional heteroatom or heteroatom containing group selected from O, NH or S, and which may be optionally substituted, one or more times, independently of each other, with R^{5A};
R^{11A} is independently selected from C₁₋₄ alkyl, C₃₋₆ cycloalkyl, C₁₋₄ haloalkyl or C₃₋₆ halocycloalkyl; 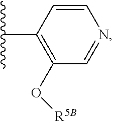
wherein
R^{5B} is C₂₋₅ alkyl optionally substituted with hydroxy, C₁₋₄ alkoxy, R^{7B}R^{8B}N—, or phenyl, wherein the phenyl group is optionally substituted with one or more times with R^{A}; or
R^{5B} is R^{6B}—CH₂—;
R^{6B} is selected from 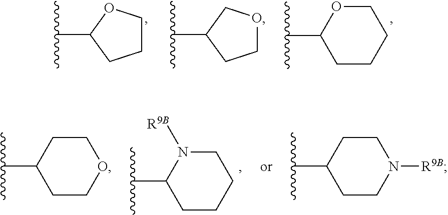
R^{7B} and R^{8B} is independently selected from C₁₋₃ alkyl, C₁₋₃ haloalkyl; or
R^{7B} and R^{8B} together with the nitrogen atom to which they are attached form a 5- to 6-membered nitrogen containing heterocyclic ring, optionally containing one additional heteroatom or heteroatom containing group selected from O and —NH—, NH(C₁₋₃ alkyl);
R^{9B} is selected from hydrogen, C₁₋₄ alkyl, or C₁₋₃ haloalkyl;
R^{5A} is selected from hydroxy, halogen, cyano, C₁₋₄-alkyl, C₁₋₄-alkoxy, C₁₋₄ haloalkyl or C₁₋₄ haloalkoxy; 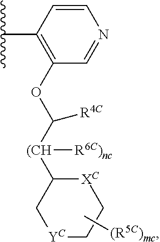
wherein
R^{4C} is selected from hydrogen or methyl;
R^{6C} is selected from hydrogen, C₁₋₃ alkyl, C₁₋₃ haloalkyl;
nc is 0 or 1;
X^{C} is NR^{7C} or O;
Y^{C} is NR^{8C} or O;
R^{7C} is methyl;
R^{8C} is selected from methyl, 2,2,2-trifloethyl, or 2,2-difluoroethyl;
R^{5C} is selected from hydrogen or methyl, wherein R^{5C} being attached to any carbon atom of the ring comprising X^{C} and Y^{C};
me is 0, 1, 2, or 3; 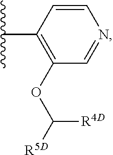
wherein
R^{4D} is selected from hydrogen or methyl;
R^{5D} is selected from the group consisting of (R/S)-2-oxetanyl, (S)-2-oxetanyl, 3-oxetanyl, (R/S)-2-azetidinyl, (S)-2-azetidinyl, 3-azetidinyl, each of which is optionally substituted one, two or three times with R⁶¹ and wherein each azetidinyl is substituted at the nitrogen with R^{8D}; OR
R^{5D} is selected from the group consisting of 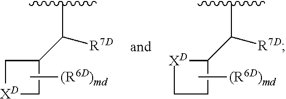
R^{6D} is selected from fluoro or C₁₋₃ alkyl;
md is selected from 0, 1, 2, or 3;
R^{7D} is selected from hydrogen, C₁₋₃ alkyl, or C₁₋₃ haloalkyl;
X^{D} is NR^{8D} of O;
R^{8D} is selected from C₁₋₃ alkyl or C₂₋₃ haloalkyl;
each occurrence of L^{g} is independently selected from the group consisting of: —O—, —NH—, —NR^{d}, —S(O)₀₋₂, C(O), and C₁₋₃ alkylene optionally substituted with from 1-3 R^{a};
each g is independently 1, 2, or 3;
each R^{g2} is a divalent R^{g} group; and
each occurrence of R′ and R″ is independently selected from the group consisting of: H; —OH; and C₁₋₄ alkyl.

77. The method of any one of claims 1-74, wherein Y is —OH.

78. The compound of claim 75 or 76, wherein Y is —OH.
